(19) United States
(12) Patent Application Publication
(10) Pub. No.: US 2026/0347487 A1
(43) Pub. Date: Nov. 5, 2026

(54) METHODS OF TREATING RHINOSINUSITIS WITH NASAL POLYPS

(71) Applicant: Upstream Bio, Inc., Waltham, MA (US)

(72) Inventor: Aaron Deykin, Needham, MA (US)

(21) Appl. No.: 19/660,739

(22) Filed: Apr. 28, 2026

Related U.S. Application Data
(60) Provisional application No. 63/796,180, filed on Apr. 28, 2025.
(60) Provisional application No. 63/873,919, filed on Sep. 1, 2025.
(60) Provisional application No. 63/961,943, filed on Jan. 16, 2026.

(51) Int. Cl. C07K 16/28; A61K 39/00; A61P 11/02

(52) U.S. Cl. CPC C07K 16/2866; A61P 11/02; A61K 2039/505; A61K 2039/54; A61K 2039/545; C07K 2317/92

(57) ABSTRACT

Methods of treating Chronic Rhinosinusitis with Nasal Polyps using anti-TSLP-R antibodies, and pharmaceutical compositions of anti-TSLP-R antibodies, are provided herein.

### CROSS-REFERENCE TO RELATED APPLICATIONS

This application claims priority to U.S. Provisional Application No. 63/796,180, filed Apr. 28, 2025, U.S. Provisional Application No. 63/873,919, filed Sep. 1, 2025, and U.S. Provisional Application No. 63/961,943, filed Jan. 16, 2026, each of which is hereby incorporated by reference in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Apr. 24, 2026, is named “UPS-009WO.xml” and is 14,286 bytes in size.

### FIELD

Embodiments of the disclosure are directed to methods for treating Chronic Rhinosinusitis with Nasal Polyps comprising administering to the subject in need thereof antibodies and antigen-binding fragments that bind to the thymic stromal lymphopoietin receptor (TSLP-R).

### BACKGROUND

Thymic stromal lymphopoietin (TSLP) is a cytokine derived from epithelial cells, and is produced in response to pro-inflammatory stimuli. TSLP interacts with the TSLP receptor (TSLP-R) and is involved in both dendritic cell mediated Th2 cell activation and direct eosinophil activation. Both TSLP and TSLP-R have been implicated in nasal polyposis and the pathogenesis of nasal polyp-associated inflammatory diseases such as Chronic Rhinosinusitis with Nasal Polyps (CRSwNP). Nasal polyposis is a T helper cell-2 (Th-2) driven inflammatory process affecting the mucosa of the nose and paranasal sinuses. Eosinophils and their products are thought to be a hallmark of nasal polyp-associated inflammation as elevated levels of interleukin-5 (IL-5; promotes eosinophil survival and differentiation), eosinophil cationic protein (ECP), and eotaxin (eosinophil chemoattractant), factors that attract and activate eosinophils, are typically found in nasal polyps. Eosinophils are the predominant inflammatory cell found in the sinuses and nasal polyps, and nasal polyps are also associated with elevated levels of IgE. TSLP and TSLP-R expression has been shown to be correlated closely with the degree of eosinophilic inflammation and local IgE level in nasal polyp tissue. CRSwNP is an inflammatory condition of the sinuses characterized by the presence of nasal polyposis and long-term symptoms including nasal obstruction, nasal congestion, reduction in or loss of sense of smell, anterior and posterior rhinorrhea, facial pain, and headache. Current medical management of CRSwNP primarily focuses on controlling inflammation, which involves administration of topical and systemic corticosteroids with a goal of reducing polyp mass and number. In case of failure, surgical excision of nasal polyps is indicated. However, disease recurrence is reported to be frequent after surgery, with rates of recurrence approaching 50% in patients with tissue eosinophilia. There is a need in the art for additional treatment methods. Accordingly, monoclonal antibody that specifically binds to human TSLP-R and inhibits the action of human TSLP through human TSLP-R is useful for preventing and/or treating CRSwNP. The present embodiments address these needs as well as others.

### SUMMARY

Embodiments herein are directed to a method of treating Chronic Rhinosinusitis with Nasal Polyps (CRSwNP) in a subject in need thereof, the method comprising administering to the subject a therapeutically effective amount of an anti-TSLP-R antibody, or antigen-binding fragment thereof.

In some embodiments, a method of treating chronic rhinosinusitis with nasal polyps (CRSwNP) in a subject in need thereof comprises administering a pharmaceutical composition comprising about 100 to about 150 mg of verekitug to the subject in need thereof. In some embodiments, the composition is administered once every twelve weeks. In some embodiments, the pharmaceutical composition comprises about 100, about 125, or about 150 mg of verekitug. In some embodiments, the pharmaceutical composition is administered subcutaneously. In some embodiments, verekitug binds to a TSLP receptor (TSLP-R). In some embodiments, verekitug blocks the formation of a heterodimeric TSLP receptor (TSLP-R). In some embodiments, the subject has a history of nasal polyp (NP) surgery or NP exacerbation requiring steroids (e.g., corticosteroids) being administered to the subject sometime in the 24 months preceding the administration of the pharmaceutical composition. In some embodiments, the subject has an endoscopic nasal polyp score (NPS)≥5. In some embodiments, the subject has a nasal congestion score (NCS)≥2 over 14 days. In some embodiments, the subject has CRSwNP symptoms for at least 8 weeks. In some embodiments, the subject in need thereof has had a stable standard of care for the treatment of CRSwNP for at least 30 days. In some embodiments, the subject in need thereof has asthma. In some embodiments, the subject is administered a second dose of verekitug at 12 weeks after the first dose. In some embodiments, the reduction in the nasal polyp score (NPS) is at least −1.0, −1.5, −2.0, or −2.5. In some embodiments, the reduction in the nasal polyp score (NPS) is measured at 24 weeks from the first dose. In some embodiments, the change of the nasal congestion score (NCS) is a reduction by at least −1.0, −1.5, or −1.75. In some embodiments, the reduction in the nasal congestion score (NCS) is measured at 24 weeks from the first dose. In some embodiments, the sinus opacification (LMK score) change from baseline is at least −2, −3, −4, −5, −6, −7, −8, or −9. In some embodiments, the sinus opacification (LMK score) change from baseline is measured at 24 weeks from the first dose. In some embodiments, the total symptom score (TSS) change from baseline is at least −7, −8, −9, or −10. In some embodiments, the TSS change from baseline is measured at 24 weeks from the first dose. In some embodiments, the difficulty with smell (DSS score) change from baseline is at least −1, −1.25, or −1.5. In some embodiments, the nasal blockage score (NBS) change from baseline is at least −1, −1.25, or −1.5. In some embodiments, the subject in need thereof treated is less likely to require sinus surgery to treat the polyps.

### BRIEF DESCRIPTION OF THE DRAWINGS

FIGS. 1A-1B: Data depicting that TRAB-1 (verekitug) produces potent suppression of TSLP/TSLP-R-mediated responses in vitro. FIG. 1A: Chart depicting cell proliferation rates in Ba/F3 cells transfected with human TSLP-R/IL-7Ra and treated with either TRAB-1 antibody, tezepelumab antibody, or IgG antibody, plotted as a function of the concentration of the antibody concentration. FIG. 1B: Bar graph depicting CCL17 (ng/mL) production in dendritic cells treated with TRAB-1 antibody, tezepelumab antibody, or IgG antibody, plotted as a function of the concentration of the antibody concentration.

FIGS. 2A-2C: Data depicting that single ascending dose (SAD) studies in healthy volunteers demonstrated sustained pharmacokinetics (PK) effect over 60 days. FIG. 2A: Graph depicting mean serum concentration over time, as a function of dose and route of administration of TRAB-1. IV=intravenous; SC=subcutaneous. FIG. 2B: Graph depicting predicted mean serum concentration of TRAB-1 over time, as a function of dose of TRAB-1 administered subcutaneously every 4 weeks. FIG. 2C: Graph depicting predicted mean serum concentration of TRAB-1 over time, as a function of dose of TRAB-1 administered subcutaneously every 12 weeks.

FIGS. 3A-3B: Data depicting that reduction of eosinophils was evident after a single dose of TRAB-1 in healthy volunteers. FIG. 3A: Graph depicting eosinophil concentrations in healthy volunteers treated with greater than or equal to 20 mg (0.3 mg/kg) of TRAB-1, as a function of time. Groups were selected based on eosinophil concentrations at day 0: <150 cells/μL; and >150 cells/μL. FIG. 3B: Graph depicting eosinophil concentrations in healthy volunteers treated with greater than or equal to 20 mg (0.3 mg/kg) of TRAB-1, as a function of time, compared to the placebo group. Groups were selected based on eosinophil concentrations at day 0: <150 cells/μL (n=17); 150-200 cells/μL (n=3); 200-300 cells/μL (n=5); and >300 cell/μL (n=5).

FIGS. 4A-4B: Data depicting an indication of pharmacokinetics/pharmacodynamics correlation in healthy volunteers treated with single ascending dose of TRAB-1. FIG. 4A: Pharmacokinetics curves depicting serum mean TRAB-1 as a function of time in healthy volunteers treated with 2 mg, 7 mg, 20 mg, 70 mg, 210 mg, or 700 mg of TRAB-1. FIG. 4B: Pharmacodynamics curves depicting eosinophils as a function of time in healthy volunteers treated with 2 mg and 7 mg, or 20 mg and 70 mg of TRAB-1.

FIG. 5: Data depicting mean serum concentrations of TRAB-1 for up to 32 weeks in asthmatic subjects that had been administered or TRAB-1 by subcutaneous (SC) injection. Of the subjects administered TRAB-1: Group 1 was administered 100 mg TRAB-1 every 4 weeks for a total of 3 administrations and measurements were monitored for up to 32 week; Group 2 was administered 200 mg TRAB-1 for a total of 2 administrations and measurements were monitored for up to 24 weeks; and Group 3 was administered 300 mg TRAB-1 for a total of 1 administration and measurements were monitored for up to 24 weeks. The serum concentrations observed were compared to the concentrations predicted from the model shown in FIG. 2B, which are indicated by the solid lines.

FIG. 6: Data depicting TSLP-R occupancy over time in asthmatic subjects that had been administered a placebo or TRAB-1 by subcutaneous (SC) injection. The placebo group was administered the placebo once and the percentage of free TSLP receptors was monitored over 24 weeks. For the subjects administered TRAB-1: Group 1 was administered 100 mg TRAB-1 every 4 weeks for a total of 2 or 3 administrations and receptor occupancy was monitored for up to 24 weeks; a Group 2 was administered 200 mg TRAB-1 for a total of 1, 2, or 3 administrations and receptor occupancy was monitored for up to 24 weeks; Group 3 was administered 300 mg TRAB-1 for a total of 1 or 2 administrations and receptor occupancy was monitored for up to 24 weeks; and Group 4 was administered a single dose of 25 mg TRAB-1 and receptor occupancy was monitored for up to 20 weeks.

FIGS. 7A-7C: Data depicting blood eosinophil concentrations over time in asthmatic subjects that had been administered a placebo or TRAB-1 by subcutaneous (SC) injection. For the subjects administered TRAB-1: Group 1 was administered 100 mg TRAB-1 every 4 weeks for a total of 2 or 3 administrations and eosinophil concentrations was monitored for up to 24 weeks; Group 2 was administered 200 mg TRAB-1 for a total of 1, 2, or 3 administrations and eosinophil concentrations was monitored for up to 24 weeks; Group 3 was administered 300 mg TRAB-1 for a total of 1 or 2 administrations and eosinophil concentrations was monitored for up to 24 weeks; and Group 4 was administered a single dose of 25 mg TRAB-1 and receptor occupancy was monitored for up to 20 weeks. FIG. 7A: Graph depicting mean blood eosinophil concentration (cells/μL) over time. Error bars represent the standard error of the mean. FIG. 7B: Graph depicting changes in mean blood eosinophil concentrations (cells/μL), compared to baseline levels, over time. Error bars represent the standard error of the mean. The dashed line represents the known maximum therapeutic response for tezepelumab (Ly et al., J Clin Pharm 2021). FIG. 7C: Graph depicting percent change in mean blood eosinophil concentrations, compared to baseline levels, over time. Error bars represent the standard error of the mean. The dashed line represents the known maximum therapeutic response for tezepelumab (Ly et al., J Clin Pharm 2021).

FIGS. 8A-8D: Data depicting fractional exhaled Nitric Oxide (FeNO) over time in asthmatic subjects that had been administered a placebo or TRAB-1 by subcutaneous (SC) injection. Of the subjects administered TRAB-1: Group 1 was administered 100 mg TRAB-1 every 4 weeks for a total of 2 or 3 administrations and FeNO was monitored for up to 32 weeks; a Group 2 was administered 200 mg TRAB-1 for a total of 1, 2, or 3 administrations and FeNO was monitored for up to 24 weeks; Group 3 was administered 300 mg TRAB-1 for a total of 1 or 2 administrations and FeNO was monitored for up to 24 weeks; and Group 4 was administered a single dose of 25 mg TRAB-1 and FeNO was monitored for up to 24 weeks. FIG. 8A: Graph depicting mean FeNO (ppb) over time. Error bars represent the standard error of the mean. FIG. 8B: Graph depicting changes in mean FeNO (ppb), compared to baseline levels, over time. Error bars represent the standard error of the mean. The dashed line represents the known maximum therapeutic response for tezepelumab (Ly et al., J Clin Pharm 2021. July; 61(7):901-912). FIG. 8C: Graph depicting percent change in mean FeNO, compared to baseline levels, over time, for Group 1, Group 2, Group 3, and Group 4. Error bars represent the standard error of the mean. The dashed line represents the known maximum therapeutic response for tezepelumab (Ly et al., J Clin Pharm 2021. July; 61(7):901-912). FIG. 8D: Graph depicting percent change in mean FeNO, compared to baseline levels, over time, for Group 1 and Group 4. Error bars represent the standard error of the mean.

FIGS. 9A-9B: Graph depicting pharmacokinetic/pharmacodynamic model of TRAB-1 serum concentration in patients treated with varying regiments of TRAB-1 FIG. 9A: Pharmacokinetic/pharmacodynamic models are depicted for TRAB-1 serum concentration in patients treated with 25 mg or 100 mg of TRAB-1 every 12 weeks (FIG. 9A) or 400 mg of TRAB-1 every 24 weeks (FIG. 9B). The threshold concentrations of TRAB-1 needed for a 50% response (EC50), 80% response (EC80), and 90% response (EC90) on FeNO, based on the MAD study, are marked. The IC90 value representing the predicted trough concentration based on the SAD study of approximately 0.3 g/mL is additionally marked.

FIG. 10: A graph depicting VIBRANT Phase 2 trial design.

FIG. 11: A graph depicting VIBRANT ITT population disposition.

FIG. 12: Tabular data showing baseline characteristics were balanced across treatment groups.

FIG. 13: Tabular data showing that verekitug was generally well tolerated.

FIG. 14: Tabular data showing that most common TEAEs in the study population were consistent with CRSwNP symptoms.

FIG. 15: Tabular data showing that verekitug led to significant and clinically meaningful improvements in NPS and key secondary endpoints, including reduction in need for surgery or systemic corticosteroids.

FIG. 16: Graphs depicting dosing of verekitug every 12 weeks leading to significant reductions in NPS and NCS over 24 weeks.

FIG. 17: Tabular data showing that verekitug demonstrated clinical effect in NPS across all subgroups.

FIG. 18: Tabular data showing that the VIBRANT population generally similar to CRSwNP studies

of other biologics.

FIG. 19: Graphs depicting comparison of verekitug dosed every 12 weeks to tezepelumab dosed every 4 weeks.

FIG. 20: Summary of top-line results for Phase 2 VIBRANT study of verekitug in CRSwNP.

### DETAILED DESCRIPTION

Provided herein are methods of treating Chronic Rhinosinusitis with Nasal Polyps (CRSwNP) by administering to a subject in need thereof antibodies, or antigen-binding fragments thereof, that specifically bind to TSLP-R. The disclosed embodiments are also directed towards methods of treating CRSwNP by administering to a subject in need thereof pharmaceutical compositions comprising anti-TSLP-R antibodies, or antigen-binding fragments thereof. In some embodiments, the anti-TSLP-R antibody, or antigen-binding fragment thereof, binds to and inhibits the activity of TSLP-R.

It is to be understood that the embodiments described herein are not limited to particular methods disclosed, as such method conditions may vary. It is also to be understood that the terminology used herein is only for the purpose of describing particular embodiments, and it is not intended to be limiting.

Furthermore, the methods described herein, unless otherwise indicated, use conventional molecular and cellular biological and immunological techniques known within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

Unless defined otherwise, all technical and scientific terms have the same meaning as it is commonly understood by one of ordinary skill in the art to which the embodiments disclosed belong. Certain terms are discussed below, or elsewhere in the specification, to provide additional guidance to the practitioner in describing the methods of the disclosure and how to use them. Moreover, it will be appreciated that the same thing can be said in more than one way. Consequently, alternative language and synonyms may be used for any one or more of the terms discussed herein, nor any special significance to be placed upon whether or not a term is elaborated or discussed herein. Synonyms for certain terms are provided. A recital of one or more synonyms does not exclude the use of other synonyms. The use of examples anywhere in the specification, including examples of any terms discussed herein, is illustrative only, and in no way limits the scope and meaning of the disclosure or any exemplified term. Likewise, the disclosure is not limited to its preferred embodiments.

Unless otherwise required by context, singular terms shall include pluralities, and plural terms shall include the singular.

The articles “a,” “an,” and “the” are used herein to refer to one or to more than one (i.e., to at least one) of the grammatical objects of the article. By way of example, “an animal” means one animal or more than one animal.

As used herein, the adverbs “about” or “approximately” mean that the numerical value is approximate and small variations would not significantly affect the practice of the disclosed embodiments. Where a numerical limitation is used, unless indicated otherwise by the context, “about” means the numerical value can vary by +5% and remain within the scope of the disclosed embodiments. Thus, about 100 means 95 to 105.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized antibodies, fully human antibodies, chimeric antibodies and camelized single domain antibodies. Another word for antibody is “immunoglobulin.” An antibody can immunospecifically bind to one or more epitope or antigen-binding site. As used herein, the assignment of amino acids to each domain within an antibody is in accordance with the Kabat numbering system.

As used herein, unless otherwise indicated, “antibody fragment” or “antigen-binding fragment” refers to antigen-binding fragments of antibodies, i.e., antibody fragments that retain the ability to bind specifically to the antigen bound by the full-length antibody, e.g., fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibody fragments.

The term “antigen” as used herein means any molecule that has the ability to generate antibodies either directly or indirectly. An “antigen” can also refer to the binding partner of an antibody, or fragment thereof. Included within the definition of “antigen” is a protein-encoding nucleic acid.

The terms “epitope” and “antigen-binding site” are meant to refer to that fragment of any molecule capable of being recognized by and bound by an antibody, or fragment thereof, at one or more of the Ab's antigen-binding regions. Epitopes usually consist of chemically active surface groupings of molecules such as amino acids or sugar side chains and have specific three-dimensional structural characteristics as well as specific charge characteristics. Examples of epitopes include, but are not limited to, the residues described herein that form TSLP-R epitopes.

As used herein, the terms “specific binding,” “immunospecific binding,” “binds immune-specifically,” and the like are used interchangeably to refer to an antibody, or fragment thereof, binding to a predetermined antigen (e.g., TSLP-R) or epitope present on the antigen. The extent of specific binding between an antibody, or fragment thereof, and its antigen can be quantified by a dissociation constant (KD), which is an equilibrium constant that measures the propensity of a complex to dissociate into smaller components. A typical KD for an antibody bound to an antigen is between about 10⁻⁹ M and about 10⁻⁶ M or less. An antibody can be defined as “specific for” a predetermined antigen if the KD between said antibody and said predetermined antigen is at least two-fold the KD between said antibody to a non-specific antigen (e.g., bovine serum albumin, casein, or another non-specific polypeptide). If the KD between an antibody and a predetermined antigen is at about 10⁻⁹ M or greater, then the antibody can be said to have “high specific binding” the predetermined antigen. Methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589 601 (1983), which are herein incorporated by reference in their entirety entirely.

The terms “purified” or “isolated” means altered or removed from the natural state. When in reference to an antibody, or fragment thereof, the terms refer to an antibody, or fragment thereof, that is substantially free of other material that associates with the molecule in its natural environment. For instance, a purified protein is substantially free of the cellular material or other proteins from the cell or tissue from which it is derived. The term refers to preparations where the isolated protein is sufficiently pure to be analyzed, or at least 70% to 80% (w/w) pure, at least 80% to 90% (w/w) pure, at least 90% to 95% pure (w/w), at least 95% pure (w/w), at least 96% pure (w/w), at least 97% pure (w/w), at least 98% pure (w/w), at least 99% pure (w/w), or 100% (w/w) pure.

As used herein, the term “encoding” refers to the inherent property of specific sequences of nucleotides in a polynucleotide, such as a gene, a cDNA, or an mRNA or viral RNA, to serve as templates for synthesis of other polymers and macromolecules in biological processes having either a defined sequence of nucleotides (i.e., rRNA, tRNA, and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “homolog” means protein sequences having between 40% and 100% sequence homology or identity to a reference sequence. Percent identity between two peptide chains can be determined by pair-wise alignment using the default settings of the AlignX module of Vector NTI v.9.0.0 (Invitrogen Corp., Carslbad, Calif).

The term “composition,” as used herein, means a product which results from the mixing or combining of more than one elements or ingredients.

The phrase “pharmaceutically acceptable” is employed herein to refer to those agents of interest/compounds, salts, compositions, pharmaceutical dosage forms, etc., which are, within the scope of sound medical judgment, suitable for use in contact with the tissues of human beings and/or other mammals without excessive toxicity, irritation, allergic response, or other problem or complication, commensurate with a reasonable benefit/risk ratio. In some embodiments, pharmaceutically acceptable means approved by a regulatory agency of the federal or a state government, or listed in the U.S. Pharmacopeia or other generally recognized pharmacopeia for use in animals (e.g., mammals), and more particularly, in humans.

The terms “patient” and “subject” are interchangeable and may be taken to mean any human which may be treated with the present composition. In some embodiments, the patient or subject is an adult, adolescent, child, or infant. In some embodiments, the patient or subject is an adolescent (i.e., 12-17 years old). In some embodiments, the patient or subject is younger than 2 years old. In some embodiments, the patient or subject is 18 years old, or older. In some embodiments, the patient or subject is between the age of 18 and 75.

As used herein, a subject that is “in need thereof” refers to a subject that has been identified as requiring treatment for the condition that is to be treated and is treated with the specific intent of treating such condition. The conditions can be, for example, any of the conditions described herein. In some embodiments, the method of treating CRSwNP in a subject in need thereof comprises administering to the subject a therapeutically effective amount of the anti-TSLP-R antibody, or antigen-binding fragment thereof, wherein the subject in need thereof has Chronic Rhinosinusitis with Nasal Polyps.

The term “disease” is used in this disclosure to mean, and is used interchangeably with, the terms disorder, condition, or illness, unless otherwise indicated.

As used herein, the terms “inhibit,” “treat,” and their conjugates means to provide a treatment. The term “treatment” includes eliciting a clinically significant response without excessive levels of side effects. The term further includes a postponement of or slowing of the development of the symptoms associated with a disease or disorder and/or a reduction in the severity of the symptoms of such disease or disorder. The term also includes preventing additional symptoms, ameliorating or preventing the underlying causes of such symptoms, and improving or ameliorating existing uncontrolled or unwanted symptoms, or otherwise obtaining beneficial or desired clinical results. Treatment also includes prolonging survival as compared to expected survival if not receiving treatment. The term “treat” can be used in reference to methods of treating specific disease, e.g., a nasal disorder or a systemic condition.

The terms “prophylaxis,” “prophylactic measures,” and “preventative measures” refer to actions taken to prevent a disease or to delay the onset of a disease, especially by specified means or against a specified disease. The terms “prevent a disease”, “disease prevention”, and the like refer to prophylactic or preventative measures prior to overt disease or disorder onset to inhibit the full development or manifestation of a disease, or to minimize the extent of the disease, or to slow its course of development.

The phrases “delay the onset of the disease,” “delay disease onset,” and the like refer to prophylactic or preventative measures prior to overt disease or disorder onset to slow its course of development or to delay disease onset compared to expected onset if not receiving prophylactic or preventative measures.

The terms “administer,” “administering,” or “administration” as used herein refer to administering a compound or agent. Treatment generally includes the administration of a compound or composition which reduces the frequency of, or delays the onset of, symptoms of a medical condition, e.g., a nasal disorder or a systemic condition. When used in reference to a nasal disorder, e.g., Chronic Rhinosinusitis with Nasal Polyps, the term “treat” additionally refers to the administration of a compound or composition treat a symptom—e.g., to improve a nasal polyp-associated parameters chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes in a subject relative to a subject not receiving the compound or composition.

As used herein, the terms “therapeutically effective amount,” “therapeutically effective dose” and “effective amount” are used interchangeably in this disclosure and refer to an amount of the antibody, or antigen-binding fragment thereof, that, when administered alone or in combination with an additional therapeutic agent to a cell, tissue, or subject, is effective to cause a measurable improvement in one or more symptom of a disease, or condition, or the progression of such disease, or condition. A therapeutically effective dose further refers to that amount of the binding compound sufficient to result in at least partial amelioration of symptoms, e.g., treatment, healing, prevention or amelioration of the relevant medical condition, or an increase in rate of treatment, healing, prevention or amelioration of such conditions. An effective amount can also result in an improvement in a subjective measure in cases where subjective measures are used to assess disease severity. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The term “route of administration” refers to the location an agent is taken up into the body and the method by which the agent is administered to said location.

The term “systemic” refers to a route of administration into the circulatory system so that the entire body is affected.

The term “local” refers to a route of administration into a specific location within the body, so that only that specific location is affected. Non-limiting examples of local administration include insufflation and topical administration. Insufflation can be used for intranasal, intrapulmonary inhalation, or intrarectal administration, or for administration to any location within the body that a gas or powder can be blown into. The term “topical” administration refers to application to a body surface such as the skin or mucous membranes. Topical medications include, but are not limited to, creams, foams, gels, lotions, shake lotions, ointments, pastes, powders, solutions, drops (e.g., ear drops and eye drops), aerosols, solids, salts, sponges, tapes, tinctures, vapors, and transdermal patches. Topical administration includes, but is not limited to, transdermal, intrarectal (suppositories), intravaginal, optical, otic, and sublingual administration. Topical compositions can be applied to body parts that include, but are not limited to, the hands, fingers, fingernails, arms, legs, feet, toes, toenails, back, chest, abdomen, buttocks, neck, face, lips, oral cavity, teeth, throat, nose, nasal cavity, ears, tympanic membrane, eyes, eyelids, sclera, scalp, genitals, vaginal cavity, anus, rectum, and any other skin or mucous membrane where a composition can be applied topically.

The term “injection” refers to administering a fluid, such as a medicament, into a part of the body by using a needle. An injection can have a systemic or a local effect. An injection can be used for intraarterial, intraarticular, intracameral, intracerebral, intradermal, intramedullary, intramuscular, intraocular/intravitreal, intraperitoneal, intrathecal/intraspinal, intravenous, intraventricular, subcutaneous, suprachoroidal, retroorbital administration, or for administration to any location within the body that can be punctured with a needle or a microneedle. An injection can also be an infusion, which refers to the continuous delivery of an agent through an intravenous or subcutaneous injection. In some embodiments, the anti-TSLP-R antibody, or antigen-binding fragment thereof, is injected.

The terms “dose” and “pharmaceutical dosage” refer to the physical form in which a drug is produced and dispensed, such as a tablet, a capsule, or an injectable. Doses can be administered one time, e.g., by continuous infusion, or by doses can be administered on a schedule, e.g., every 4 hours, daily, 1-7 times per week, weekly, bi-weekly, monthly, bimonthly, quarterly, semiannually, annually etc.

The terms “in combination with” and “co-administration” as used herein means two or more agents can be administered to an animal or subject together in a mixture, concurrently as single agents or sequentially as single agents in any order. Unless otherwise described, the terms as used herein refer to the administration of an anti-TSLP-R antibody, or antigen-binding fragment thereof, and the administration of a second therapeutic agent. Agents can be administered by the same or different route of administration or at the same or different time. In some embodiments, the anti-TSLP-R antibody, or antigen-binding fragment thereof, is administered in combination with a therapeutic agent that is injected. In some embodiments, the anti-TSLP-R antibody, or antigen-binding fragment thereof, is administered in combination with a therapeutic agent that is administered topically, orally, or by injection.

### Anti-TSLP-R Antibodies

Thymic stromal lymphopoietin (TSLP) is a cytokine produced in and secreted from epithelial cells in response to pro-inflammatory stimuli. TSLP enhances the allergic inflammatory response through the activation of dendritic cells and mast cells (Liu, Yong-Jun. Adv Immunol. 2009; 101:1-25). TSLP activates dendritic cells by binding to TSLP-R/IL7R-α heterodimers composed of one TSLP receptor (TSLP-R) and one IL-7 receptor α-chain (IL7R-α). The dendritic cells activated by TSLP express inflammatory cytokines that induce the differentiation of naive T cells into Th2 cells and additionally attract Th2 cells to inflammation sites (Nat. Immunol., 2002, Vol. 7, p. 673 to 680; Int. Immunol., 1999, Vol. 11, p. 81 to 88).

In some embodiments, an anti-TSLP-R antibody, or antigen-binding fragment thereof, is provided. In some embodiments, the anti-TSLP-R antibody, or antigen-binding fragment thereof, comprises one or more peptides having the sequences as listed in Table 1 or a variant thereof.

TABLE 1

| AB ID NO. | LC Sequence | HC Sequence |
|---|---|---|
| TRAB-1 | DIQMTQSPSSLSASVGDRVTI | EVQLLESGGGLVQPGGSLRLSCAASGFTFRSSAMHWVRQAPG |
| | TCRASQDISNYLAWFQQKPGK | KGLKWVSSVSGSGAGTYYADSVKGRFTISRDNPKNTLYLQMN |
| | APKSLIYTASSLQSGVPSKFS | SLRAEDTAVYYCVKEGGSRGFDYWGQGTLVTVSSASTKGPSV |
| | GSGSGTDFTLTISSLQPEDFA | FPLAPSSKSTSGGTAALGCLVKDYFPEPVTVSWNSGALTSGV |
| | TYYCQQYNLYPPTFGQGTKVE | HTFPAVLQSSGLYSLSSVVTVPSSSLGTQTYICNVNHKPSNT |
| | IKRTVAAPSVFIFPPSDEQLK | KVDKKVEPKSCDKTHTCPPCPAPELLGGPSVFLFPPKPKDTL |
| | SGTASVVCLLNNFYPREAKVQ | MISRTPEVTCVVVDVSHEDPEVKFNWYVDGVEVHNAKTKPRE |
| | WKVDNALQSGNSQESVTEQDS | EQYNSTYRVVSVLTVLHQDWLNGKEYKCKVSNKALPAPIEKT |
| | KDSTYSLSSTLTLSKADYEKH | ISKAKGQPREPQVYTLPPSRDELTKNQVSLTCLVKGFYPSDI |
| | KVYACEVTHQGLSSPVTKSEN | AVEWESNGQPENNYKTTPPVLDSDGSFFLYSKLTVDKSRWQQ |
| | RGEC | GNVFSCSVMHEALHNHYTQKSLSLSPGK |
| | (SEQ ID NO: 3) | (SEQ ID NO: 1) |

In some embodiments, the anti-TSLP-R antibody, or antigen-binding fragment thereof, comprises one or more heavy chain (HC). In some embodiments, the anti-TSLP-R antibody, or antigen-binding fragment thereof, comprises one or more heavy chain having the amino acid sequence of SEQ ID NO: 1. In some embodiments, the anti-TSLP-R antibody, or antigen-binding fragment thereof, comprises two heavy chains having the amino acid sequence of SEQ ID NO: 1. In some embodiments, the anti-TSLP-R antibody, or antigen-binding fragment thereof, comprises a heavy chain encoded by the nucleotide sequence of SEQ ID NO: 2:

(SEQ ID NO: 2)
gaggtgcagctgttggagtctgggggaggcttggtacagccgggggggtc
cctgagactctcctgtgcagcctctggattcacctttcgcagctctgcca
tgcattgggtccgccaggctccagggaagggactgaaatgggtctcaagt
gttagtggcagtggtgctggaacatactacgcagactccgtgaagggccg
gttcaccatctccagagacaatcccaagaatacactgtatctgcaaatga
acagtctgagagccgaggacacggccgtatattattgtgtgaaagaaggg
ggcagccggggttttgactactggggccagggaaccctggtcaccgtctc
ctcagcctccaccaagggcccatcggtcttccccctggcaccctcctcca
agagcacctctgggggcacagcggccctgggctgcctggtcaaggactac
ttccccgaaccggtgacggtgtcgtggaactcaggcgccctgaccagcgg
cgtgcacaccttcccggctgtcctacagtcctcaggactctactccctta
gtagcgtggtgaccgtgccctccagcagcttgggcacccagacctacatc
tgcaacgtgaatcacaagcccagcaacaccaaggtggacaagaaagttga
gcccaaatcttgtgacaaaactcacacatgcccaccgtgcccagcacctg
aactcctggggggaccgtcagtcttcctcttccccccaaaacccaaggac
accctcatgatctcccggacccctgaggtcacatgcgtggtggtggacgt
gagccacgaagaccctgaggtcaagttcaactggtacgtggacggcgtgg
aggtgcataatgccaagacaaagccgcgggaggagcagtacaacagcacg
taccgtgtggtcagcgtcctcaccgtcctgcaccaggactggctgaatgg
caaggagtacaagtgcaaggtctccaacaaagccctcccagcccccatcg
agaaaaccatctccaaagccaaagggcagccccgagaaccacaggtgtac
accctgcccccatcccgggatgagctgaccaagaaccaggtcagcctgac
ctgcctggtcaaaggcttctatcccagcgacatcgccgtggagtgggaga
gcaatgggcagccggagaacaactacaagaccacgcctcccgtgctggac
tccgacggctccttcttcctctacagcaagctcaccgtggacaagagcag
gtggcagcaggggaacgtcttctcatgctccgtgatgcatgaggctctgc
acaaccactacacgcagaagagcctctccctgtctccgggtaaatga.

In some embodiments, the anti-TSLP-R antibody, or antigen-binding fragment thereof, comprises a heavy chain having the amino acid sequence of SEQ ID NO: 1, encoded by the nucleotide sequence of SEQ ID NO: 2.

In some embodiments, the anti-TSLP-R antibody, or antigen-binding fragment thereof, comprises one or more light chain (LC). In some embodiments, the anti-TSLP-R antibody, or antigen-binding fragment thereof, comprises one or more light chain having the amino acid sequence of SEQ ID NO: 3. In some embodiments, the anti-TSLP-R antibody, or antigen-binding fragment thereof, comprises two light chains having the amino acid sequence of SEQ ID NO: 3. In some embodiments, the anti-TSLP-R antibody, or antigen-binding fragment thereof, comprises a light chain encoded by the nucleotide sequence of SEQ ID NO: 4:

(SEQ ID NO: 4)
gacatccagatgacccagtctccatcctcactgtctgcatctgtaggaga
cagagtcaccatcacttgtcgggcgagtcaggacattagcaattatttag
cctggtttcagcagaaaccagggaaagcccctaagtccctgatctatact
gcatccagtttgcaaagtggggtcccatcaaagttcagcggcagtggatc
tgggacagatttcactctcaccatcagcagcctgcagcctgaagattttg
caacttattactgccaacagtataatctttatcctccgacgttcggccaa
gggaccaaggtggaaatcaaacggactgtggctgcaccatctgtcttcat
cttcccgccatctgatgagcagttgaaatctggaactgcctctgttgtgt
gcctgctgaataacttctatcccagagaggccaaagtacagtggaaggtg
gataacgccctccaatcgggtaactcccaggagagtgtcacagagcagga
cagcaaggacagcacctacagcctcagcagcaccctgacgctgagcaaag
cagactacgagaaacacaaagtctacgcctgcgaagtcacccatcagggc
ctgagctcgcccgtcacaaagagcttcaacaggggagagtgttag.

In some embodiments, the anti-TSLP-R antibody, or antigen-binding fragment thereof, comprises a light chain having the amino acid sequence of SEQ ID NO: 3, encoded by the nucleotide sequence of SEQ ID NO: 4.

In some embodiments, the anti-TSLP-R antibody, or antigen-binding fragment thereof, comprises one or more heavy chain having the amino acid sequence of SEQ ID NO: 1 encoded by the nucleotide sequence of SEQ ID NO: 2, and one or more light chain having the amino acid sequence of SEQ ID NO: 3 encoded by the nucleotide sequence of SEQ ID NO: 4. In some embodiments, the anti-TSLP-R antibody, or antigen-binding fragment thereof, comprises two heavy chains having the amino acid sequence of SEQ ID NO: 1 encoded by the nucleotide sequence of SEQ ID NO: 2, and two light chains having the amino acid sequence of SEQ ID NO: 3 encoded by the nucleotide sequence of SEQ ID NO: 4.

In some embodiments, the anti-TSLP-R antibody, or antigen-binding fragment thereof, comprises a heavy chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1. In some embodiments, the anti-TSLP-R antibody, or antigen-binding fragment thereof, comprises a light chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 3. In some embodiments, the anti-TSLP-R antibody, or antigen-binding fragment thereof, comprises a heavy chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1; and a light chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 3.

In some embodiments, the anti-TSLP-R antibody, or antigen-binding fragment thereof, comprises one or more heavy chain variable regions (V_{H}). In some embodiments, the anti-TSLP-R antibody, or antigen-binding fragment thereof, comprises one or more light chain variable regions (V_{L}). In some embodiments, the antibody heavy chain sequence comprises the heavy chain variable region of the antibody, and the antibody light chain sequence comprises the light chain variable region of the antibody. In some embodiments, the anti-TSLP-R antibody, or antigen-binding fragment thereof, comprises a heavy chain variable region V_{H} and the light chain variable region V_{L}, having the sequences shown in Table 2.

TABLE 2

| AB ID NO. | V_{L} Sequence | V_{H} Sequence |
|---|---|---|
| TRAB-1 | DIQMTQSPSSLSASVGDRVTITCRASQ | EVQLLESGGGLVQPGGSLRLSCAASGFTFRSSAMHWV |
| | DISNYLAWFQQKPGKAPKSLIYTASSL | RQAPGKGLKWVSSVSGSGAGTYYADSVKGRFTISRDN |
| | QSGVPSKFSGSGSGTDFTLTISSLQPE | PKNTLYLQMNSLRAEDTAVYYCVKEGGSRGFDYWGQG |
| | DFATYYCQQYNLYPPTFGQGTKVEIK | TLVTVSS |
| | (SEQ ID NO: 6) | (SEQ ID NO: 5) |

In some embodiments, the anti-TSLP-R antibody, or antigen-binding fragment thereof, comprises a heavy chain variable region having an amino acid sequence of SEQ ID NO: 5. In some embodiments, the anti-TSLP-R antibody, or antigen-binding fragment thereof, comprises a light chain variable region having the amino acid sequence of SEQ ID NO: 6. In some embodiments, the anti-TSLP-R antibody, or antigen-binding fragment thereof, comprises a heavy chain variable region having the amino acid sequence of SEQ ID NO: 5, and a light chain variable region having the amino acid sequence of SEQ ID NO: 6.

In some embodiments, the anti-TSLP-R antibody, or antigen-binding fragment thereof, comprises a heavy chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 5. In some embodiments, the anti-TSLP-R antibody, or antigen-binding fragment thereof, comprises a light chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 6. In some embodiments, the anti-TSLP-R antibody, or antigen-binding fragment thereof, comprises a heavy chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 5; and a light chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 6.

In some embodiments, the anti-TSLP-R antibody, or antigen-binding fragment thereof, comprises a CDR selected from Table 3. As used herein, the terms LCDR1, LCDR2, and LCDR3 refer to the CDR1, CDR2, and CDR3 of the light chain. As used herein, the terms HCDR1, HCDR2, and HCDR3 refer to the CDR1, CDR2, and CDR3 of the heavy chain. In some embodiments, the anti-TSLP-R antibody, or antigen-binding fragment thereof, contains one or more CDR regions. In some embodiments, the anti-TSLP-R antibody, or antigen-binding fragment thereof, contains one or more CDR region with a sequence selected from Table 3.

TABLE 3

| Ab ID No | LCDR1 | LCDR2 | LCDR3 | HCDR1 | HCDR2 | HCDR3 |
|---|---|---|---|---|---|---|
| TRAB-1 | RASQDISNY | TASSLQS | QQYNLYPPT | SSAMH | SVSGSGAGT | EGGSRGFDY |
| | LA | (SEQ ID | (SEQ ID | (SEQ ID | YYADSVKG | (SEQ ID |
| | (SEQ ID | NO: 11) | NO: 12) | NO: 7) | (SEQ ID | NO: 9) |
| | NO: 10) | | | | NO: 8) | |

In some embodiments, the anti-TSLP-R antibody, or antigen-binding fragment thereof, comprises a heavy chain variable region comprising a HCDR1 having the amino acid sequence of SEQ ID NO: 7; a HCDR2 having the amino acid sequence of SEQ ID NO: 8; and a HCDR3 having the amino acid sequence of SEQ ID NO: 9. In some embodiments, the anti-TSLP-R antibody, or antigen-binding fragment thereof, comprises a heavy chain variable region comprising a HCDR1 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 7; a HCDR2 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 8; and a HCDR3 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 9.

In some embodiments, the anti-TSLP-R antibody, or antigen-binding fragment thereof, comprises a heavy chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 5, provided that the CDRs comprises a HCDR1 amino acid sequence of SEQ ID NO: 7; a HCDR2 amino acid sequence of SEQ ID NO: 8; and a HCDR3 amino acid sequence of SEQ ID NO: 9.

In some embodiments, the anti-TSLP-R antibody, or antigen-binding fragment thereof, comprises a light chain variable region comprising a LCDR1 having the amino acid sequence of SEQ ID NO: 10; a LCDR2 having the amino acid sequence of SEQ ID NO: 11; and a LCDR3 having the amino acid sequence of SEQ ID NO: 12. In some embodiments, the anti-TSLP-R antibody, or antigen-binding fragment thereof, comprises a light chain variable region comprising a LCDR1 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 10; a LCDR2 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 11; and a LCDR3 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 12.

In some embodiments, the anti-TSLP-R antibody, or antigen-binding fragment thereof, comprises a light chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 6, provided that the light chain variable region comprises a LCDR1 amino acid sequence of SEQ ID NO: 10; a LCDR2 amino acid sequence of SEQ ID NO: 11; and a LCDR3 amino acid sequence of SEQ ID NO: 12. In some embodiments, the anti-TSLP-R antibody, or antigen-binding fragment thereof, comprises a variable light chain variable region comprising a LCDR1 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 10; a LCDR2 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 11; and a LCDR3 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 12.

In some embodiments, the anti-TSLP-R antibody, or antigen-binding fragment thereof, comprises a heavy chain variable region comprising a HCDR1 having the amino acid sequence of SEQ ID NO: 7; a HCDR2 having the amino acid sequence of SEQ ID NO: 8; and a HCDR3 having the amino acid sequence of SEQ ID NO: 9; and a light chain variable region comprising a LCDR1 having the amino acid sequence of SEQ ID NO: 10; a LCDR2 having the amino acid sequence of SEQ ID NO: 11; and a LCDR3 having the amino acid sequence of SEQ ID NO: 12.

In some embodiments, the anti-TSLP-R antibody, or antigen-binding fragment thereof, comprises a heavy chain variable region comprising a HCDR1 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 7; a HCDR2 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 8; and a HCDR3 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 9; and a light chain variable region comprising a LCDR1 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 10; a LCDR2 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 11; and a LCDR3 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 12.

In some embodiments, the anti-TSLP-R antibody, or antigen-binding fragment thereof, comprises a heavy chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 5, provided that the CDRs comprises a HCDR1 amino acid sequence of SEQ ID NO: 7; a HCDR2 amino acid sequence of SEQ ID NO: 8; and a HCDR3 amino acid sequence of SEQ ID NO: 9; and a light chain variable region having an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 6, provided that the light chain variable region comprises a LCDR1 amino acid sequence of SEQ ID NO: 10; a LCDR2 amino acid sequence of SEQ ID NO: 11; and a LCDR3 amino acid sequence of SEQ ID NO: 12.

In some embodiments, the anti-TSLP-R antibody, or antigen-binding fragment thereof, comprises a heavy chain comprising a HCDR1 having the amino acid sequence of SEQ ID NO: 7; a HCDR2 having the amino acid sequence of SEQ ID NO: 8; and a HCDR3 having the amino acid sequence of SEQ ID NO: 9; and a light chain comprising a LCDR1 having the amino acid sequence of SEQ ID NO: 10; a LCDR2 having the amino acid sequence of SEQ ID NO: 11; and a LCDR3 having the amino acid sequence of SEQ ID NO: 12.

In some embodiments, the anti-TSLP-R antibody, or antigen-binding fragment thereof, comprises a heavy chain comprising a HCDR1 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 7; a HCDR2 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 8; and a HCDR3 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 9; and a light chain comprising a LCDR1 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 10; a LCDR2 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 11; and a LCDR3 having the amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 12.

In some embodiments, the anti-TSLP-R antibody, or antigen-binding fragment thereof, is verekitug. In some embodiments, verekitug and TRAB-1 can be used interchangeably. In some embodiments, verekitug comprises a heavy chain having an amino acid sequence of SEQ ID NO: 1. In some embodiments, verekitug comprises a light chain having an amino acid sequence of SEQ ID NO: 3. In some embodiments, verekitug comprises a heavy chain having an amino acid sequence of SEQ ID NO: 1; and a light chain having an amino acid sequence of SEQ ID NO: 3.

In some embodiments, the anti-TSLP-R antibody, or antigen-binding fragment thereof, has conservative substitutions as compared to a sequence described herein. Exemplary conservative substitutions are illustrated in Table 4 and are encompassed within the scope of the disclosed subject matter. In some embodiments, the conservative substitution may reside in the framework regions, or in antigen-binding sites, as long they do not adversely affect the properties of the antibody, or fragment thereof. In some embodiments, substitutions may be made to improve the properties or the antibody, or fragment thereof, for example the stability or affinity. In some embodiments, conservative substitutions will produce molecules having functional and chemical characteristics similar to those molecules into which such modifications are made. Exemplary amino acid substitutions are shown in the table below.

TABLE 4
Exemplary Conservative Substitutions

| Original Residue | Exemplary Conservative Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

In some embodiments, variants of the proteins and peptides provided herein are provided. In some embodiments, the variant comprises a substitution, deletions, or insertion. In some embodiments, the variant comprises 1, 2, 3, 4, 5, 6, 7, 8, 9, or 10 (e.g., 1-10) substitutions. In some embodiments, the substitution can be a conservative substitution. In some embodiments, the substitution is non-conservative. In some embodiments, the variant comprises 1, 2, 3, 4, 5, 6, 7, 8, 9, or 10 (e.g., 1-10) deletions. In some embodiments, the variant comprises 1, 2, 3, 4, 5, 6, 7, 8, 9, or 10 (e.g., 1-10) insertions. In some embodiments, the substitutions, deletions, or insertions are present in the CDRs provided for herein. In some embodiments, the substitutions, deletions, or insertions are not present in the CDRs provided for herein.

In some embodiments, the variant comprises 1 substitution. In some embodiments, the variant comprises 2 substitutions. In some embodiments, the variant comprises 3 substitutions. In some embodiments, the variant comprises 4 substitutions. In some embodiments, the variant comprises 5 substitutions. In some embodiments, the variant comprises 6 substitutions. In some embodiments, the variant comprises 7 substitutions. In some embodiments, the variant comprises 8 substitutions. In some embodiments, the variant comprises 9 substitutions. In some embodiments, the variant comprises 10 substitutions. In some embodiments, the variant comprises 1 deletion. In some embodiments, the variant comprises 2 deletions. In some embodiments, the variant comprises 3 deletions. In some embodiments, the variant comprises 4 deletions. In some embodiments, the variant comprises 5 deletions. In some embodiments, the variant comprises 6 deletions. In some embodiments, the variant comprises 7 deletions. In some embodiments, the variant comprises 8 deletions. In some embodiments, the variant comprises 9 deletions. In some embodiments, the variant comprises 10 deletions. In some embodiments, the variant comprises 1 insertion. In some embodiments, the variant comprises 2 insertions. In some embodiments, the variant comprises 3 insertions. In some embodiments, the variant comprises 4 insertions. In some embodiments, the variant comprises 5 insertions. In some embodiments, the variant comprises 6 insertions. In some embodiments, the variant comprises 7 insertions. In some embodiments, the variant comprises 8 insertions. In some embodiments, the variant comprises 9 insertions. In some embodiments, the variant comprises 10 insertions.

In some embodiments, the substitutions, deletions, or insertions are present in the CDRs provided for herein. In some embodiments, the substitutions, deletions, or insertions are not present in the CDRs provided for herein.

In some embodiments, the anti-TSLP-R antibody, or antigen-binding fragment thereof, can include, but is not limited to, a monoclonal, polyclonal, bispecific, humanized, fully human, chimeric, or a single-domain antibody, or fragment thereof, or any combination thereof.

In some embodiments, the anti-TSLP-R antibody fragment can include, but is not limited to, a Fab, Fab′, F(ab′)2, Fv fragment, diabody, linear antibody, single chain antibody, sc-Fv, nanobody, or multispecific antibody fragment, or any combination thereof.

In some embodiments, the anti-TSLP-R antibody, or antigen-binding fragment thereof, is a MAb which binds to TSLP-R. In some embodiments, the anti-TSLP-R antibody, or antigen-binding fragment thereof, is a human IgG. In some embodiments, the anti-TSLP-R antibody, or antigen-binding fragment thereof, is a variant of human IgG. A “variant of human IgG” refers to an antibody that has been modified to be a human IgG or fragment thereof, when the starting antibody is not a human IgG antibody, or fragment thereof. In some embodiments, the sequences of the antibodies, or antigen-binding fragments thereof, can be modified to yield human IgG antibodies, or antigen-binding fragments thereof. In some embodiments, the conversion of the sequences provided herein can be modified to yield other types of antibodies, or antigen-binding fragments thereof. In some embodiments, the CDRs can also be linked to other antibodies, or antigen-binding fragments thereof, that bind to TSLP-R. In some embodiments, the CDRs can also be linked to other proteins or molecules to create an antibody, or fragment thereof, that binds to TSLP-R.

In some embodiments, the variable regions of the anti-TSLP-R antibody, or antigen-binding fragment thereof, as described herein can be combined with any type of constant region including a human constant region or murine constant region. In some embodiments, the constant region comprises a mutation at position at an amino acid residue relative to a wild-type human IgG constant domain, numbered according to the EU numbering index of Kabat. In some embodiments, the mutation increases the half-life of the anti-TSLP-R antibody, or antigen-binding fragment thereof, as compared to the half-life of an IgG having the wild-type human IgG constant domain.

In some embodiments, the anti-TSLP-R antibody, or antigen-binding fragment thereof, comprise an Fc region. In some embodiments, the Fc region comprises a mutation that extends the half-life of the anti-TSLP-R antibody, or antigen-binding fragment thereof, when linked to the Fc region. In some embodiments, the Fc domain comprises a mutation such as those described in US2007041972A1, EP2235059B1, U.S. Pat. Nos. 7,670,600, 8,394,925, and Mueller et al, Mol Immunol 1997 April; 34(6):441-52, each of which is incorporated by reference in its entirety. The numbering referenced herein refers to the Kabat numbering system for the Fc region.

As described herein, it is also intended that an antibody, or fragment thereof, of the invention can include conservative or non-conservative amino acid substitutions, which can also be referred to as “conservative variants” or “function conserved variants” of the antibody, or fragment thereof, that do not substantially alter its biological activity. In some embodiments, a variant antibody, or fragment thereof, or antigen-binding fragment of the antibodies, thereof, provided herein retains at least 10% of its TSLP-R binding activity (when compared to a parental antibody, or fragment thereof, that is modified) when that activity is expressed on a molar basis. In some embodiments, a variant antibody, or fragment thereof, provided herein, retains at least 20%, 50%, 70%, 80%, 90%, 95% or 100% or more of the TSLP-R binding affinity as the parental antibody, or fragment thereof.

In some embodiments, the anti-TSLP-R antibody, or antigen-binding fragment thereof, that binds to an epitope on a TSLP-R protein, is capable of inhibiting TSLP-R function. In some embodiments, the anti-TSLP-R antibody, or antigen-binding fragment thereof, displays in vitro and/or in vivo TSLP-R inhibiting or therapeutic activities. In some embodiments, the anti-TSLP-R antibody, or antigen-binding fragment thereof, is suitable as a therapeutic agent for treating TSLP-R-associated conditions in humans and animals.

As used herein, the terms “TSLP-R associated pathology,” “TSLP-R mediated disease,” and the like refer to conditions that are associated with or mediated by TSLP-R activity.

Non-limiting examples of TSLP-R associated pathologies include Chronic Rhinosinusitis with Nasal Polyps and the like. In some embodiments, the TSLP-R associated pathology is Chronic Rhinosinusitis with Nasal Polyps. In some embodiments, a therapeutically effective amount of the anti-TSLP-R antibody, or antigen-binding fragment thereof, is used to treat Chronic Rhinosinusitis with Nasal Polyps in a subject in need thereof. In some embodiments, administration of a therapeutically effective amount of the anti-TSLP-R antibody, or antigen-binding fragment thereof, to a subject in need thereof, is used to treat Chronic Rhinosinusitis with Nasal Polyps in the subject in need thereof.

### Pharmaceutical Compositions, Pharmaceutical Dosage, and Routes of Administration

In some embodiments, the anti-TSLP-R antibody, or antigen-binding fragment thereof, is formulated as a pharmaceutical composition. In some embodiments, the pharmaceutical composition comprises an anti-TSLP-R antibody, or antigen-binding fragment thereof, as disclosed herein. In some embodiments, the pharmaceutical composition comprises the anti-TSLP-R antibody, or antigen-binding fragment thereof, at a concentration from about 1 mg/mL to about 1000 mg/mL. In some embodiments, the pharmaceutical composition comprises the anti-TSLP-R antibody, or antigen-binding fragment thereof, at a concentration from about 1 mg/mL to about 900 mg/mL. In some embodiments, the pharmaceutical composition comprises the anti-TSLP-R antibody, or antigen-binding fragment thereof, at a concentration from about 1 mg/mL to about 800 mg/mL. In some embodiments, the pharmaceutical composition comprises the anti-TSLP-R antibody, or antigen-binding fragment thereof, at a concentration from about 1 mg/mL to about 700 mg/mL. In some embodiments, the pharmaceutical composition comprises the anti-TSLP-R antibody, or antigen-binding fragment thereof, at a concentration from about 1 mg/mL to about 300 mg/mL. In some embodiments, the pharmaceutical composition comprises the anti-TSLP-R antibody, or antigen-binding fragment thereof, at a concentration from about 1 mg/mL to about 500 mg/mL. In some embodiments, the pharmaceutical composition comprises the anti-TSLP-R antibody, or antigen-binding fragment thereof, at a concentration from about 1 mg/mL to about 400 mg/mL. In some embodiments, the pharmaceutical composition comprises the anti-TSLP-R antibody, or antigen-binding fragment thereof, at a concentration from about 1 mg/mL to about 300 mg/mL. In some embodiments, the pharmaceutical composition comprises the anti-TSLP-R antibody, or antigen-binding fragment thereof, at a concentration from about 50 mg/mL to about 250 mg/mL. In some embodiments, the pharmaceutical composition comprises the anti-TSLP-R antibody, or antigen-binding fragment thereof, at a concentration from about 100 mg/mL to about 200 mg/mL. In some embodiments, the pharmaceutical composition comprises the anti-TSLP-R antibody, or antigen-binding fragment thereof, at a concentration from about 150 mg/mL to about 200 mg/mL.

In some embodiments, the anti-TSLP-R antibody, or antigen-binding fragment thereof, is formulated as a pharmaceutical composition, wherein the pharmaceutical composition is any one pharmaceutical composition as provided in any one of U.S. Pat. Nos. 12,150,991; 12,447,210; 11,712,472; 10,994,011; or U.S. Publication No. 20250064929, each of which is hereby incorporated by reference in its entirety.

In some embodiments, the pharmaceutical composition comprises the anti-TSLP-R antibody, or antigen-binding fragment thereof, at a concentration of about 25 mg/mL. In some embodiments, the pharmaceutical composition comprises the anti-TSLP-R antibody, or antigen-binding fragment thereof, at a concentration of about 50 mg/mL. In some embodiments, the pharmaceutical composition comprises the anti-TSLP-R antibody, or antigen-binding fragment thereof, at a concentration of about 75 mg/mL. In some embodiments, the pharmaceutical composition comprises the anti-TSLP-R antibody, or antigen-binding fragment thereof, at a concentration of about 100 mg/mL. In some embodiments, the pharmaceutical composition comprises the anti-TSLP-R antibody, or antigen-binding fragment thereof, at a concentration of about 125 mg/mL. In some embodiments, the pharmaceutical composition comprises the anti-TSLP-R antibody, or antigen-binding fragment thereof, at a concentration of about 150 mg/mL. In some embodiments, the pharmaceutical composition comprises the anti-TSLP-R antibody, or antigen-binding fragment thereof, at a concentration of about 155 mg/mL. In some embodiments, the pharmaceutical composition comprises the anti-TSLP-R antibody, or antigen-binding fragment thereof, at a concentration of about 160 mg/mL. In some embodiments, the pharmaceutical composition comprises the anti-TSLP-R antibody, or antigen-binding fragment thereof, at a concentration of about 165 mg/mL. In some embodiments, the pharmaceutical composition comprises the anti-TSLP-R antibody, or antigen-binding fragment thereof, at a concentration of about 170 mg/mL. In some embodiments, the pharmaceutical composition comprises the anti-TSLP-R antibody, or antigen-binding fragment thereof, at a concentration of about 175 mg/mL. In some embodiments, the pharmaceutical composition comprises the anti-TSLP-R antibody, or antigen-binding fragment thereof, at a concentration of about 180 mg/mL. In some embodiments, the pharmaceutical composition comprises the anti-TSLP-R antibody, or antigen-binding fragment thereof, at a concentration of about 185 mg/mL. In some embodiments, the pharmaceutical composition comprises the anti-TSLP-R antibody, or antigen-binding fragment thereof, at a concentration of about 190 mg/mL. In some embodiments, the pharmaceutical composition comprises the anti-TSLP-R antibody, or antigen-binding fragment thereof, at a concentration of about 195 mg/mL. In some embodiments, the pharmaceutical composition comprises the anti-TSLP-R antibody, or antigen-binding fragment thereof, at a concentration of about 200 mg/mL. In some embodiments, the pharmaceutical composition comprises the anti-TSLP-R antibody, or antigen-binding fragment thereof, at a concentration of about 205 mg/mL. In some embodiments, the pharmaceutical composition comprises the anti-TSLP-R antibody, or antigen-binding fragment thereof, at a concentration of about 210 mg/mL. In some embodiments, the pharmaceutical composition comprises the anti-TSLP-R antibody, or antigen-binding fragment thereof, at a concentration of about 215 mg/mL. In some embodiments, the pharmaceutical composition comprises the anti-TSLP-R antibody, or antigen-binding fragment thereof, at a concentration of about 220 mg/mL. In some embodiments, the pharmaceutical composition comprises the anti-TSLP-R antibody, or antigen-binding fragment thereof, at a concentration of about 225 mg/mL. In some embodiments, the pharmaceutical composition comprises the anti-TSLP-R antibody, or antigen-binding fragment thereof, at a concentration of about 230 mg/mL. In some embodiments, the pharmaceutical composition comprises the anti-TSLP-R antibody, or antigen-binding fragment thereof, at a concentration of about 235 mg/mL. In some embodiments, the pharmaceutical composition comprises the anti-TSLP-R antibody, or antigen-binding fragment thereof, at a concentration of about 240 mg/mL. In some embodiments, the pharmaceutical composition comprises the anti-TSLP-R antibody, or antigen-binding fragment thereof, at a concentration of about 245 mg/mL. In some embodiments, the pharmaceutical composition comprises the anti-TSLP-R antibody, or antigen-binding fragment thereof, at a concentration of about 250 mg/mL. In some embodiments, the pharmaceutical composition comprises the anti-TSLP-R antibody, or antigen-binding fragment thereof, at a concentration of about 255 mg/mL. In some embodiments, the pharmaceutical composition comprises the anti-TSLP-R antibody, or antigen-binding fragment thereof, at a concentration of about 260 mg/mL. In some embodiments, the pharmaceutical composition comprises the anti-TSLP-R antibody, or antigen-binding fragment thereof, at a concentration of about 265 mg/mL. In some embodiments, the pharmaceutical composition comprises the anti-TSLP-R antibody, or antigen-binding fragment thereof, at a concentration of about 270 mg/mL. In some embodiments, the pharmaceutical composition comprises the anti-TSLP-R antibody, or antigen-binding fragment thereof, at a concentration of about 275 mg/mL. In some embodiments, the pharmaceutical composition comprises the anti-TSLP-R antibody, or antigen-binding fragment thereof, at a concentration of about 280 mg/mL. In some embodiments, the pharmaceutical composition comprises the anti-TSLP-R antibody, or antigen-binding fragment thereof, at a concentration of about 285 mg/mL. In some embodiments, the pharmaceutical composition comprises the anti-TSLP-R antibody, or antigen-binding fragment thereof, at a concentration of about 290 mg/mL. In some embodiments, the pharmaceutical composition comprises the anti-TSLP-R antibody, or antigen-binding fragment thereof, at a concentration of about 300 mg/mL. In some embodiments, the pharmaceutical composition comprises the anti-TSLP-R antibody, or antigen-binding fragment thereof, at a concentration of about 325 mg/mL. In some embodiments, the pharmaceutical composition comprises the anti-TSLP-R antibody, or antigen-binding fragment thereof, at a concentration of about 350 mg/mL. In some embodiments, the pharmaceutical composition comprises the anti-TSLP-R antibody, or antigen-binding fragment thereof, at a concentration of about 375 mg/mL. In some embodiments, the pharmaceutical composition comprises the anti-TSLP-R antibody, or antigen-binding fragment thereof, at a concentration of about 400 mg/mL. In some embodiments, the pharmaceutical composition comprises the anti-TSLP-R antibody, or antigen-binding fragment thereof, at a concentration of about 425 mg/mL. In some embodiments, the pharmaceutical composition comprises the anti-TSLP-R antibody, or antigen-binding fragment thereof, at a concentration of about 450 mg/mL. In some embodiments, the pharmaceutical composition comprises the anti-TSLP-R antibody, or antigen-binding fragment thereof, at a concentration of about 475 mg/mL. In some embodiments, the pharmaceutical composition comprises the anti-TSLP-R antibody, or antigen-binding fragment thereof, at a concentration of about 500 mg/mL. In some embodiments, the pharmaceutical composition comprises the anti-TSLP-R antibody, or antigen-binding fragment thereof, at a concentration of about 525 mg/mL. In some embodiments, the pharmaceutical composition comprises the anti-TSLP-R antibody, or antigen-binding fragment thereof, at a concentration of about 550 mg/mL. In some embodiments, the pharmaceutical composition comprises the anti-TSLP-R antibody, or antigen-binding fragment thereof, at a concentration of about 575 mg/mL. In some embodiments, the pharmaceutical composition comprises the anti-TSLP-R antibody, or antigen-binding fragment thereof, at a concentration of about 600 mg/mL. In some embodiments, the pharmaceutical composition comprises the anti-TSLP-R antibody, or antigen-binding fragment thereof, at a concentration of about 700 mg/mL. In some embodiments, the pharmaceutical composition comprises the anti-TSLP-R antibody, or antigen-binding fragment thereof, at a concentration of about 800 mg/mL. In some embodiments, the pharmaceutical composition comprises the anti-TSLP-R antibody, or antigen-binding fragment thereof, at a concentration of about 900 mg/mL. In some embodiments, the pharmaceutical composition comprises the anti-TSLP-R antibody, or antigen-binding fragment thereof, at a concentration of about 1000 mg/mL.

In some embodiments, the pharmaceutical composition comprises: (i) an anti-TSLP-R antibody, or antigen-binding fragment thereof, at a concentration of about 1 mg/mL to about 300 mg/mL; (ii) a pharmaceutically acceptable buffer at a concentration of about 5 mmol/L to about 100 mmol/L; (iii) arginine, or a pharmaceutically acceptable salt thereof, at a concentration of about 1 mmol/L to about 600 mmol/L; and (iv) a surfactant at a concentration of about 0.001% to about 1% (w/v), wherein the pharmaceutically acceptable buffer is histidine buffer, and wherein the anti-TSLP-R antibody, or antigen-binding fragment thereof, is verekitug. In some embodiments, the pharmaceutical composition comprises: (i) an anti-TSLP-R antibody, or antigen-binding fragment thereof, at a concentration of about 1 mg/mL to about 300 mg/mL; (ii) a pharmaceutically acceptable buffer at a concentration of about 5 mmol/L to about 100 mmol/L; (iii) arginine, or a pharmaceutically acceptable salt thereof, at a concentration of 700 mmol/L or less; and (iv) a surfactant at a concentration of about 0.001% to about 1% (w/v), wherein the pharmaceutically acceptable buffer is histidine buffer, and wherein the anti-TSLP-R antibody, or antigen-binding fragment thereof, is verekitug. In some embodiments, the pharmaceutical composition comprises the anti-human TSLP receptor antibody, or an antigen-binding fragment thereof, at a concentration of 1 mg/mL to 200 mg/mL; the arginine, or a pharmaceutically acceptable salt thereof, at a concentration of no more than 210 mmol/L; the surfactant is present in an amount of 0.01 to 0.2% (w/v), wherein the surfactant is polysorbate 80; and the pharmaceutically acceptable buffer is histidine at a concentration of 5 to 50 mmol/L, wherein the anti-TSLP-R antibody, or antigen-binding fragment thereof, is verekitug. In some embodiments, the pharmaceutical composition comprises an anti-human TSLP receptor antibody, a pharmaceutically acceptable buffer, arginine or a pharmaceutically acceptable salt thereof, and a surfactant, wherein the anti-TSLP-R antibody, or antigen-binding fragment thereof, is verekitug, wherein a concentration of the arginine or a pharmaceutically acceptable salt thereof is 700 mmol/L or less, wherein the pharmaceutical composition is a liquid preparation, or a lyophilized preparation or a spray-dried preparation, wherein when the pharmaceutical composition is a liquid preparation, a content of the anti-human TSLP receptor antibody is 0.007 to 2 mmol/L, or when the pharmaceutical composition is a lyophilized preparation or a spray-dried preparation, a content of a solution after reconstituting the preparation in water is 0.007 to 2 mmol/L, and wherein when the pharmaceutical composition is a liquid preparation, a pH of the liquid preparation is 5 to 6, or when the pharmaceutical composition is a lyophilized preparation or a spray-dried preparation, a pH of a solution after reconstituting the preparation in water is 5 to 6. In some embodiments, the pharmaceutical composition comprises an anti-human TSLP receptor antibody, or an antigen-binding fragment thereof, at a concentration of 1 mg/mL to 300 mg/mL; a pharmaceutically acceptable buffer at a concentration of 5 to 100 mmol/L; arginine, or a pharmaceutically acceptable salt thereof, at a concentration of 700 mmol/L or less; and a surfactant present in an amount of 0.001 to 1% (w/v), wherein the anti-TSLP-R antibody, or antigen-binding fragment thereof, is verekitug. In some embodiments, the pharmaceutical composition comprises an anti-human TSLP receptor antibody, a pharmaceutically acceptable buffer, arginine or a pharmaceutically acceptable salt thereof, and a surfactant, wherein the following (1) and/or (2) is contained as the anti-human TSLP receptor antibody: (1) an anti-human TSLP receptor antibody comprising a heavy chain consisting of the amino acid sequence of SEQ ID NO: 1, and a light chain consisting of the amino acid sequence of SEQ ID NO: 3, and (2) an anti-human TSLP receptor antibody consisting of amino acid sequences of an antibody generated by a post-translational modification of the anti-human TSLP receptor antibody of (1), wherein the post-translational modification is a pyroglutamylation at the N-terminus of a heavy chain variable region and/or lysine deletion at the C-terminus of a heavy chain, wherein a concentration of the arginine or a pharmaceutically acceptable salt thereof is 700 mmol/L or less, wherein the pharmaceutical composition is a liquid preparation, or a lyophilized preparation or a spray-dried preparation, wherein when the pharmaceutical composition is a liquid preparation, a content of the anti-human TSLP receptor antibody is 0.007 to 2 mmol/L, or when the pharmaceutical composition is a lyophilized preparation or a spray-dried preparation, a content of a solution after reconstituting the preparation in water is 0.007 to 2 mmol/L, and wherein when the pharmaceutical composition is a liquid preparation, a pH of the liquid preparation is 5 to 6, or when the pharmaceutical composition is a lyophilized preparation or a spray-dried preparation, a pH of a solution after reconstituting the preparation in water is 5 to 6. In some embodiments, the pharmaceutical composition comprises an anti-human TSLP receptor antibody, or an antigen-binding fragment thereof, at a concentration of 1 mg/mL to 300 mg/mL; a pharmaceutically acceptable buffer at a concentration of 5 to 100 mmol/L; arginine, or a pharmaceutically acceptable salt thereof, at a concentration of 700 mmol/L or less; and a surfactant present in an amount of 0.001 to 1% (w/v), wherein the anti-human TSLP receptor antibody, or an antigen-binding fragment thereof, comprises a heavy chain comprising the amino acid sequence of SEQ ID NO: 1, or a heavy chain comprising the amino acid sequence of SEQ ID NO: 1 with a posttranslational modification, and a light chain having the amino acid sequence of SEQ ID NO: 3, and wherein the posttranslational modification of the heavy chain is a lysine deletion at the C-terminus of the heavy chain having the amino acid sequence of SEQ ID NO: 1, or a pyroglutamylation at the N-terminus of the heavy chain having the amino acid sequence of SEQ ID NO: 1.

In some embodiments, the pharmaceutical composition comprises: (i) an anti-TSLP-R antibody, or antigen-binding fragment thereof, at a concentration of about 1 mg/mL to about 300 mg/mL; (ii) a pharmaceutically acceptable buffer at a concentration of about 5 mmol/L to about 100 mmol/L; (iii) glycine, or a pharmaceutically acceptable salt thereof, at a concentration of about 1 mmol/L to about 600 mmol/L; and (iv) a surfactant at a concentration of about 0.001% to about 1% (w/v), wherein the pharmaceutically acceptable buffer is histidine buffer. In some embodiments, the pharmaceutical composition comprises: (i) an anti-TSLP-R antibody, or antigen-binding fragment thereof, at a concentration of about 150 mg/mL to about 300 mg/mL; (ii) a pharmaceutically acceptable buffer at a concentration of about 5 mmol/L to about 100 mmol/L; (iii) glycine, or a pharmaceutically acceptable salt thereof, at a concentration of about 150 mmol/L to about 200 mmol/L; and (iv) a surfactant at a concentration of about 0.001% to about 1% (w/v), wherein the pharmaceutically acceptable buffer is histidine buffer, and wherein the anti-TSLP-R antibody, or antigen-binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 7; the heavy chain CDR2 has the amino acid sequence of SEQ ID NO: 8; and the heavy chain CDR3 sequence has the amino acid sequence of SEQ ID NO: 9; and (ii) a light chain variable region comprising light chain CDR1, CDR2, and CDR3 sequences, wherein the light chain CDR1 sequence has the amino acid sequence SEQ ID NO: 10; the light chain CDR2 sequence has the amino acid sequence of SEQ ID NO: 11; and the light chain CDR3 sequence has the amino acid sequence of SEQ ID NO: 12. In some embodiments, the pharmaceutical composition comprises: (i) an anti-TSLP-R antibody, or antigen-binding fragment thereof, at a concentration of about 150 mg/mL to about 300 mg/mL; (ii) a pharmaceutically acceptable buffer at a concentration of about 5 mmol/L to about 100 mmol/L; (iii) glycine, or a pharmaceutically acceptable salt thereof, at a concentration of about 150 mmol/L to about 200 mmol/L; and (iv) a surfactant at a concentration of about 0.001% to about 1% (w/v), wherein the pharmaceutically acceptable buffer is histidine buffer, and wherein the anti-TSLP-R antibody, or antigen-binding fragment thereof, is verekitug. In some embodiments, the pharmaceutical composition comprises: (i) an anti-TSLP-R antibody, or antigen-binding fragment thereof, at a concentration of about 150 mg/mL to about 300 mg/mL; (ii) histidine buffer at a concentration of about 10 mmol/L to about 30 mmol/L; (iii) glycine, or a pharmaceutically acceptable salt thereof, at a concentration of about 150 mmol/L to about 200 mmol/L; (iv) polysorbate 80 at a concentration of about 0.01% to about 0.05% (w/v); and (v) a pH of about 5.7, wherein the anti-TSLP-R antibody, or antigen-binding fragment thereof, is verekitug. In some embodiments, the pharmaceutical composition comprises: (i) an anti-TSLP-R antibody, or antigen-binding fragment thereof, at a concentration of about 150 mg/mL to about 300 mg/mL; (ii) histidine buffer at a concentration of 18 mmol/L to 22 mmol/L; (iii) glycine, or a pharmaceutically acceptable salt thereof, at a concentration of 160 mmol/L to 200 mmol/L; (iv) polysorbate 80 at a concentration of 0.01% (w/v) to 0.05% (w/v); and (v) a pH of 5.6 to 5.8, wherein the anti-TSLP-R antibody, or antigen-binding fragment thereof, is verekitug. In some embodiments, the pharmaceutical composition comprises: (i) an anti-TSLP-R antibody, or antigen-binding fragment thereof, at a concentration of about 150 mg/mL to about 300 mg/mL; (ii) histidine buffer at a concentration of about 20 mmol/L; (iii) glycine, or a pharmaceutically acceptable salt thereof, at a concentration of about 180 mmol/L; (iv) polysorbate 80 at a concentration of about 0.03% (w/v); and (v) a pH of about 5.7, wherein the anti-TSLP-R antibody, or antigen-binding fragment thereof, is verekitug. In some embodiments, the pharmaceutical composition comprises: (i) an anti-TSLP-R antibody, or antigen-binding fragment thereof, at a dose of 0.03 mg/kg to 10 mg/kg; (ii) histidine buffer at a concentration of about 20 mmol/L; (iii) glycine, or a pharmaceutically acceptable salt thereof, at a concentration of about 180 mmol/L; (iv) polysorbate 80 at a concentration of about 0.03% (w/v); and (v) a pH of about 5.7, wherein the pharmaceutically acceptable buffer is histidine buffer, wherein the anti-TSLP-R antibody, or antigen-binding fragment thereof, is verekitug. In some embodiments, the pharmaceutical composition comprises histidine buffer at a concentration of 18 mmol/L to 22 mmol/L; glycine, or a pharmaceutically acceptable salt thereof, at a concentration of 160 mmol/L to 200 mmol/L; the surfactant is polysorbate 80 at a concentration of 0.01% (w/v) to 0.05% (w/v); and the pharmaceutical composition is at a pH of about 5.6 to 5.8, wherein the anti-TSLP-R antibody, or antigen-binding fragment thereof, is verekitug. In some embodiments, the pharmaceutical composition comprises histidine buffer at a concentration of 20 mmol/L; glycine, or a pharmaceutically acceptable salt thereof, at a concentration of 180 mmol/L; the surfactant is polysorbate 80 at a concentration of 0.03% (w/v); and the pharmaceutical composition is at a pH of about 5.7, wherein the anti-TSLP-R antibody, or antigen-binding fragment thereof, is verekitug.

In some embodiments, the pharmaceutical composition comprises: (i) an anti-TSLP-R antibody, or antigen-binding fragment thereof, at a concentration of about 1 mg/mL to about 300 mg/mL; (ii) a pharmaceutically acceptable buffer at a concentration of about 5 mmol/L to about 100 mmol/L; (iii) proline, or a pharmaceutically acceptable salt thereof, at a concentration of about 1 mmol/L to about 600 mmol/L; and (iv) a surfactant at a concentration of about 0.001% to about 1% (w/v), wherein the pharmaceutically acceptable buffer is histidine buffer.

In some embodiments, the pharmaceutical composition comprises: (i) an anti-TSLP-R antibody, or antigen-binding fragment thereof, at a concentration of about 1 mg/mL to about 300 mg/mL; (ii) a pharmaceutically acceptable buffer at a concentration of about 5 mmol/L to about 100 mmol/L; (iii) proline or a pharmaceutically acceptable salt thereof, at a concentration of 150 mmol/L to about 200 mmol/L; and (iv) a surfactant at a concentration of about 0.001% to about 1% (w/v); wherein the anti-TSLP-R antibody, or antigen-binding fragment thereof, comprises: (i) a heavy chain variable region comprising heavy chain CDR1, CDR2, and CDR3 sequences, wherein the heavy chain CDR1 sequence has the amino acid sequence of SEQ ID NO: 7; the heavy chain CDR2 has the amino acid sequence of SEQ ID NO: 8; and the heavy chain CDR3 sequence has the amino acid sequence of SEQ ID NO: 9; and (ii) a light chain variable region comprising light chain CDR1, CDR2, and CDR3 sequences, wherein the light chain CDR1 sequence has the amino acid sequence SEQ ID NO: 10; the light chain CDR2 sequence has the amino acid sequence of SEQ ID NO: 11; and the light chain CDR3 sequence has the amino acid sequence of SEQ ID NO: 12. In some embodiments, the pharmaceutical composition comprises: (i) an anti-TSLP-R antibody, or antigen-binding fragment thereof, at a concentration of about 1 mg/mL to about 300 mg/mL; (ii) a pharmaceutically acceptable buffer at a concentration of about 5 mmol/L to about 100 mmol/L; (iii) proline or a pharmaceutically acceptable salt thereof, at a concentration of 150 mmol/L to about 200 mmol/L; and (iv) a surfactant at a concentration of about 0.001% to about 1% (w/v); wherein the anti-TSLP-R antibody, or antigen-binding fragment thereof, is verekitug. In some embodiments, the pharmaceutical composition comprises: (i) an anti-TSLP-R antibody, or antigen-binding fragment thereof, at a concentration of about 150 mg/mL to about 300 mg/mL; (ii) a pharmaceutically acceptable buffer at a concentration of about 5 mmol/L to about 100 mmol/L; (iii) proline or a pharmaceutically acceptable salt thereof, at a concentration of 150 mmol/L to about 200 mmol/L; and (iv) a surfactant at a concentration of about 0.001% to about 1% (w/v); wherein the anti-TSLP-R antibody, or antigen-binding fragment thereof, is verekitug. In some embodiments, the pharmaceutical composition comprises: histidine buffer at a concentration of 18 mmol/L to 22 mmol/L; proline, or a pharmaceutically acceptable salt thereof, at a concentration of 160 mmol/L to 200 mmol/L; the surfactant is polysorbate 80 at a concentration of 0.01% (w/v) to 0.05% (w/v); and the pharmaceutical composition is at a pH of about 5.6 to 5.8; wherein the anti-TSLP-R antibody, or antigen-binding fragment thereof, is verekitug. In some embodiments, the pharmaceutical composition comprises: histidine buffer at a concentration of 20 mmol/L; proline, or a pharmaceutically acceptable salt thereof, at a concentration of 180 mmol/L; the surfactant is polysorbate 80 at a concentration of 0.02% (w/v); and the pharmaceutical composition is at a pH of about 5.7; wherein the anti-TSLP-R antibody, or antigen-binding fragment thereof, is verekitug.

In some embodiments, the pharmaceutical composition comprising the anti-TSLP-R antibody, or antigen-binding fragment thereof, is administered to a subject in accordance with the Physicians' Desk Reference 2003 (Thomson Healthcare; 57th edition (Nov. 1, 2002)). Treatment of individuals may comprise the administration of a therapeutically effective amount of the antibodies, or fragments thereof, as described herein.

Pharmaceutical compositions can be administered with medical devices known in the art. For example, a pharmaceutical composition of the invention can be administered by injection with a hypodermic needle, including, e.g., a prefilled syringe or autoinjector. The pharmaceutical compositions may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556, each of which is hereby incorporated by reference in its entirety.

The pharmaceutical compositions may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer the pharmaceutical composition in a local rather than systemic manner, for example, via injection of the antibody, or fragment thereof, directly into an arthritic joint or pathogen-induced lesion characterized by immunopathology, often in a depot or sustained release formulation. Furthermore, one may administer the antibody, or fragment thereof, in a targeted drug delivery system, for example, in a liposome coated with a tissue-specific antibody, or fragment thereof, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, or fragment thereof, the level of symptoms, the immunogenicity of the therapeutic antibody, or fragment thereof, and the accessibility of the target cells in the biological matrix. Preferably, the administration regimen delivers sufficient therapeutic antibody, or fragment thereof, to effect improvement in the target disease state, while simultaneously minimizing undesired side effects. Accordingly, the amount of biologic delivered depends in part on the particular therapeutic antibody, or fragment thereof, and the severity of the condition being treated. Guidance in selecting appropriate doses of therapeutic antibodies, or fragments thereof, is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

The pharmaceutical compositions provided herein can be provided by continuous infusion, or by doses administered, e.g., daily, weekly, monthly, bimonthly, quarterly, semiannually, etc. Doses may be provided, e.g., intravenously or subcutaneously.

Methods of Treating Chronic Rhinosinusitis with Nasal Polyps

In some embodiments, the anti-TSLP-R antibodies, or antigen-binding fragments thereof, are suitable as therapeutic agents for treating TSLP-R-associated conditions in humans. An antibody, or fragment thereof, capable treating a condition associated with TSLP-R activity, or used to treat a TSLP-R associated pathology, is intended to be provided to subjects in an amount sufficient to affect a reduction, resolution, or amelioration in the TSLP-R related symptom or pathology.

Accordingly, in some embodiments, methods of treating a TSLP-R associated pathology in a subject in need thereof are provided. In some embodiments, the methods of treating a TSLP-R associated pathology comprise administering to the subject an antibody, or an antigen-binding fragment thereof, that specifically binds to TSLP-R. In some embodiments, the methods of treating a TSLP-R associated pathology comprise administering to the subject a therapeutically effective amount of an anti-TSLP-R antibody, or antigen-binding fragment thereof, as provided for herein. In some embodiments, the anti-TSLP-R antibody, or antigen-binding fragment thereof, is provided for herein. In some embodiments, the methods of treating a TSLP-R associated pathology comprise administering a pharmaceutical composition comprising an anti-TSLP-R antibody, or antigen-binding fragment thereof, as provided for herein. In some embodiments, the pharmaceutical composition comprising the anti-TSLP-R antibody, or antigen-binding fragment thereof, is provided for herein.

In some embodiments, the anti-TSLP-R antibody, or antigen-binding fragment thereof, is an antibody capable of treating a condition associated with a TSLP-R activity. In some embodiments, the anti-TSLP-R antibody, or antigen-binding fragment thereof, is provided to subjects in an amount sufficient to produce a reduction, resolution, or amelioration of the TSLP-R associated pathology, as provided for herein. In some embodiments, the methods of treating a TSLP-R associated pathology comprise administering a therapeutically or a prophylactically effective amount of the anti-TSLP-R antibodies, or antigen-binding fragments thereof, as described herein, to a susceptible subject or to a subject exhibiting a condition in which TSLP-R is known or suspected to have caused the observed pathology.

In some embodiments, the TSLP-R associated pathology is Chronic Rhinosinusitis with Nasal Polyps.

As used herein, the term “sinusitis” refers to any inflammatory condition characterized by inflammation of the paranasal sinuses, including inflammation of the maxillary, frontal, ethmoid and/or sphenoid paranasal sinuses. Acute sinusitis is characterized by a sudden onset of cold-like symptoms such as runny, stuffy nose and facial pain that does not go away after 10 to 14 days. Acute sinusitis typically lasts less than four weeks. Sub-acute sinusitis lasts four to eight weeks.

As used herein, “chronic sinusitis” (“CS”) refers to an inflammatory condition of the sinuses characterized by any combination of specific symptoms including, but not limited to, nasal congestion, decreased or lost sense of smell, anterior and/or posterior nasal discharge, facial pain, and/or headache and consequences thereof, often for a period of years. A formal diagnosis is made on the basis of sinus computed tomography (CT) scan and/or sinus endoscopy. Based on endoscopic evaluation, CS can be clinically divided as CS with nasal polyps/nasal polyposis (CRSwNP) or CS without nasal polyps/nasal polyposis. Chronic sinusitis lasts eight weeks or longer, and recurrent sinusitis is characterized by sinusitis episodes that occur three or more times in one year. Many patients with chronic sinusitis have “chronic hyperplastic eosinophilic sinusitis,” which is characterized by marked inflammation of the sinuses, increased eosinophils and mixed mononuclear cells, and a relative paucity of neutrophils. Some of these patients have one or more of associated nasal polyps, asthma, and aspirin or NSAID sensitivity.

As used herein, “allergic sinusitis” refers to sinusitis that occurs when the body's immune system responds to specific, non-infectious irritants such as e.g., plant pollens, molds, dust mites, animal hair, industrial chemicals (including tobacco smoke), foods, medicines, and insect venom. As used herein, “non-allergic sinusitis” refers to sinusitis that is not due to an allergic reaction, e.g., from colds, allergies, and tissue irritants (e.g., nasal sprays, cocaine, cigarette smoke and the like). Less commonly, sinuses can become obstructed by tumors or growths.

The term “rhinitis” refers to an allergic response, such as to a common allergen (“allergic rhinitis,” e.g., perennial allergic rhinitis) or to an environmental irritant (“non-allergic rhinitis”). Symptoms of allergic rhinitis include sneezing; stuffy or runny nose; sinus pressure, and pain or throbbing in the cheeks or nose; and itching in the nose, throat, eyes and ears. As used herein, “allergic rhinitis” refers to rhinitis that occurs when the body's immune responds to specific, non-infectious irritants. As used herein, “non-allergic rhinitis” refers to rhinitis that is not due to an allergic reaction, that can be triggered by factors such as, e.g., cigarette smoke and other pollutants, strong odors, strong chemical environments, alcoholic beverages, cold, blockages in the nose, a deviated septum, infections and over-use of medications such as decongestants and/or nasal sprays. Symptoms of non-allergic rhinitis include constriction or inflammation in the nasal passages which leads to many of the same symptoms of allergic rhinitis.

As used herein, the term “rhinosinusitis” refers to a condition that has symptoms of both rhinitis and sinusitis. Rhinosinusitis includes acute rhinosinusitis and chronic rhinosinusitis. Acute rhinosinusitis can be caused by an infection, such as a bacterial, viral or fungal infection, or by a chemical irritation. Cigarette-smoke-induced acute rhinosinusitis and chlorine fume-induced chronic rhinosinusitis are examples of acute rhinosinusitis. Nasal polyposis is most commonly associated with chronic rhinosinusitis (CRS), which is characterized by mucosal inflammation of the nasal cavity and paranasal sinuses with symptoms lasting more than 8 weeks. Chronic eosinophilic rhinosinusitis with nasal polyps is a condition that lasts longer than 8 weeks.

Chronic sinusitis (CS) and chronic rhinosinusitis (CRS) are conditions that last longer than eight weeks. The underlying causes of acute sinusitis and acute rhinosinusitis may lead to chronic sinusitis or chronic rhinosinusitis if the resulting inflammation persists for more than 8 weeks. Chronic rhinosinusitis is rhinitis together with inflammation and blockage in the sinuses; this can be brought on by an infection, by growths in the sinuses (nasal polyps) or by swelling of the lining of the sinuses. Chronic rhinosinusitis includes for example, eosinophilic chronic hyperplastic rhinosinusitis.

Additional subcategories of chronic sinusitis (and chronic rhinosinusitis) include, e.g., superantigen-induced eosinophilic chronic sinusitis (e.g., sinusitis induced by exo- and endo-toxins produced by bacteria such as Staphylococcus aureus); allergic fungal sinusitis (e.g., sinusitis induced by fungi such as Aspergillus or Alternaria); non-allergic fungal eosinophilic chronic sinusitis; and aspirin-exacerbated eosinophilic chronic sinusitis. Rhinosinusitis is further classified as allergic rhinosinusitis and non-allergic rhinosinusitis.

As used herein, “allergic rhinosinusitis” refers to rhinosinusitis that occurs in response to exposure to one or more allergens. As used herein, “non-allergic rhinosinusitis” refers to rhinosinusitis caused by factors including, but not limited to: pregnancy; thyroid disorders as a side effect of certain blood pressure and/or topical OTC decongestant medications; structural abnormalities in the nasal septum, structural abnormalities in the nasal filtering structures (turbinates), and/or structural abnormalities in the sinus drainage tracts; nasal polyps; and eosinophils.

As used herein, a “nasal polyp” is an overgrowth of tissue in one or more of the nasal cavities. The condition of having nasal polyps is called “nasal polyposis.” Risk factors for nasal polyposis include genetic susceptibility, anatomic abnormality, mucociliary impairment, infection, and local immunologic imbalance. Nasal polyposis is associated with sinusitis (e.g., allergic or non-allergic sinusitis), rhinitis (e.g., allergic and non-allergic rhinitis), rhinosinusitis (e.g., allergic or non-allergic rhinosinusitis), asthma (e.g., moderate-to-severe asthma), NSAID sensitivity (e.g., aspirin sensitivity), and infection, such as bacterial and fungal infection. About 80% of nasal polyps are highly edematous and filled with eosinophils. Nasal polyps can also present as fibrous, glandular or cystic. Nasal polyps are typically teardrop-shaped growths that form in the nose and/or sinuses, obstructing the sinuses and nasal passages. Nasal polyposis is a clinical condition characterized by the presence of multiple polyps in the upper nasal cavity, originating from the osteomeatal complex. Nasal polyposis is characterized by long-term symptoms of nasal obstruction and congestion, reduction in or loss of sense of smell, anterior and posterior rhinorrhea, and facial pain. The presence or absence of nasal polyps can be confirmed for example by performing endoscopy, and the presence and extent of sinus and polyp involvement can be confirmed by methods such as coronal computed tomography (CT) scans. Nasal polyposis is a common finding in patients with chronic rhinosinusitis. More than 80% of patients with nasal polyps/nasal polyposis have eosinophilic upper airway inflammation. Nasal polyposis is a T helper cell-2 (Th-2) driven inflammatory process affecting the mucosa of the nose and paranasal sinuses. Eosinophils and their products are thought to be a hallmark of nasal polyp-associated inflammation as elevated levels of interleukin-5 (IL-5; promotes eosinophil survival and differentiation), ECP, and eotaxin (eosinophil chemoattractant), factors that attract and activate eosinophils, are typically found in nasal polyps. Eosinophils are the predominant inflammatory cell found in the sinuses and nasal polyps, and nasal polyps are also associated with elevated levels of IgE. Nasal polyposis is associated with elevated blood eosinophil counts, elevated FeNO levels, elevated serum IL-5 levels, and elevated serum IL-13 levels in asthma patients. As used herein, “bilateral nasal polyposis” refers to the presence of one or more NPs at each side of the nasal cavity.

A subject with nasal polyposis, such as a patient with chronic sinusitis with bilateral nasal polyposis, can have concomitant Samter's triad (defined by present of nasal polyposis, asthma, and aspirin and NSAID sensitivity). A subject with nasal polyposis can also have concomitant asthma, and/or other Th2 concomitant systemic conditions. Chronic Rhinosinusitis with Nasal Polyps/Nasal Polyposis (CRSwNP) is highly comorbid with asthma. Approximately 7% of all asthma patients and 25-35% of severe asthma patients have CRSwNP, and approximately up to 60% of CRSwNP patients have asthma. Bacterial infections include, for example, staphylococcus infections. A subject with nasal polyposis can have a chronic infection, such as a chronic bacterial infection, e.g., a chronic Staphylococcus aureus infection.

Chronic Rhinosinusitis with Nasal Polyps/Nasal Polyposis (CRSwNP) occurs when the nose sinuses are swollen and inflamed for three months or longer, despite treatment. CRSwNP is an inflammatory condition of the sinuses characterized by the presence of nasal polyposis and long-term symptoms including nasal obstruction, nasal congestion, reduction in or loss of sense of smell, anterior and posterior rhinorrhea, facial pain, and headache. Current medical management of CRSwNP primarily focuses on controlling inflammation, which involves administration of topical and systemic corticosteroids with a goal of reducing polyp mass and number. In case of failure, surgical excision of nasal polyps (surgical polypectomy) is indicated. However, disease recurrence is reported to be frequent after surgery, with rates of recurrence approaching 50% in patients with tissue eosinophilia.

Without wishing to be bound by a particular theory, the cause of CRSwNP is multifactorial, with genetic and environmental factors, and impaired immune response. The inflammatory component is thought to be mediated primarily by the Th2 type T-cell activation pathway. TSLP and TSLP-R, which are associated with CD4+ T cell mediated Th2-type inflammation, have both been implicated in the pathogenesis of CRSwNP and other respiratory diseases such as asthma. Genetic variants located in or near TSLP have been detected in genome wide studies for rhinitis, and TSLP polymorphisms have been found to associated with a higher risk of allergic rhinitis (Ramasamy A et al, J Allergy Clin Immunol. 2011 November; 128(5):996-1005). Elevated levels of TSLP mRNA has been detected in nasal polyposis tissue in patients with CRSwNP when compared with healthy sinus tissue from healthy controls, and when compared to sinus tissue from patients with CRS but without nasal polyps, suggesting that TSLP activity is increased in nasal polyps of patients with chronic rhinosinusitis (Kimura S et al, Allergy Asthma Immunol Res. 2011; 3(3):186-193; Nagarka D R et al, J Allergy Clin Immunol. 2013 September; 132(3):593-600.e12). A strong correlation has also been shown between TSLP protein levels, TSLP mRNA levels, and eosinophilic inflammation in CRSwNP. The presence of TSLP correlates with the number of eosinophils in nasal polyp tissue, and the activity of TSLP in polyp tissue is believed to be sufficient to trigger an inflammatory response.

Taken together, these data suggest a role for TSLP in the pathogenesis of Chronic Rhinosinusitis with Nasal Polyps. Targeting TSLP-TSLP-R signaling via pharmacological inhibition or by antibody mediated neutralization of TSLP-R could be a beneficial tool to prevent and treat CRSwNP associated with elevated TSLP. There remains a need for methods of treating Chronic Rhinosinusitis with Nasal Polyps using pharmaceutical compositions that contain antibodies and fragments thereof, that are specific for TSLP-R.

In some embodiments, the anti-TSLP-R antibody, or antigen-binding fragment thereof, is an antibody capable of treating Chronic Sinusitis with Nasal Polyps/Nasal Polyposis (CRSwNP). In some embodiments, methods of treating Chronic Rhinosinusitis with Nasal Polyps are provided herein. In some embodiments, the anti-TSLP-R antibody, or antigen-binding fragment thereof, may be used to treat, or reduce the severity of, Chronic Rhinosinusitis with Nasal Polyps or a symptom thereof. In some embodiments, the pharmaceutical compositions comprising the anti-TSLP-R antibodies, or antigen-binding fragments thereof, may be used to treat, or reduce the severity of, Chronic Rhinosinusitis with Nasal Polyps or a symptom thereof.

In some embodiments, the anti-TSLP-R antibody, or antigen-binding fragment thereof, described herein is indicated for treatment of sinusitis. In some embodiments, the anti-TSLP-R antibody, or antigen-binding fragment thereof, described herein is indicated for treatment of chronic sinusitis. In some embodiments, the anti-TSLP-R antibody, or antigen-binding fragment thereof, described herein is indicated for treatment of chronic hyperplastic eosinophilic sinusitis. In some embodiments, the anti-TSLP-R antibody, or antigen-binding fragment thereof, described herein is indicated for treatment of allergic sinusitis. In some embodiments, the anti-TSLP-R antibody, or antigen-binding fragment thereof, described herein is indicated for treatment of non-allergic sinusitis. In some embodiments, the anti-TSLP-R antibody, or antigen-binding fragment thereof, described herein is indicated for treatment of rhinitis. In some embodiments, the anti-TSLP-R antibody, or antigen-binding fragment thereof, described herein is indicated for treatment of allergic rhinitis. In some embodiments, the anti-TSLP-R antibody, or antigen-binding fragment thereof, described herein is indicated for treatment of non-allergic rhinitis. In some embodiments, the anti-TSLP-R antibody, or antigen-binding fragment thereof, described herein is indicated for treatment of rhinosinusitis. In some embodiments, the anti-TSLP-R antibody, or antigen-binding fragment thereof, described herein is indicated for treatment of chronic rhinosinusitis. In some embodiments, the anti-TSLP-R antibody, or antigen-binding fragment thereof, described herein is indicated for treatment of eosinophilic chronic hyperplastic rhinosinusitis. In some embodiments, the anti-TSLP-R antibody, or antigen-binding fragment thereof, described herein is indicated for treatment of non-allergic rhinosinusitis. In some embodiments, the anti-TSLP-R antibody, or antigen-binding fragment thereof, described herein is indicated for treatment of nasal polyposis. In some embodiments, the anti-TSLP-R antibody, or antigen-binding fragment thereof, described herein is indicated for treatment of bilateral nasal polyposis. In some embodiments, the anti-TSLP-R antibody, or antigen-binding fragment thereof, described herein is indicated for treatment of nasal polyposis associated with acute sinusitis, sub-acute sinusitis, chronic sinusitis, recurrent sinusitis, rhinitis, chronic rhinitis, rhinosinusitis, or chronic rhinosinusitis. In some embodiments, the anti-TSLP-R antibody, or antigen-binding fragment thereof, described herein is indicated for treatment of nasal polyposis in a subject who has chronic hyperplastic eosinophilic sinusitis. In some embodiments, the anti-TSLP-R antibody, or antigen-binding fragment thereof, described herein is indicated for the long-term treatment of adults with chronic sinusitis with bilateral nasal polyposis.

In some embodiments, the anti-TSLP-R antibody, or antigen-binding fragment thereof, described herein is indicated for treatment of Chronic Rhinosinusitis with Nasal Polyps (CRSwNP). In some embodiments, the anti-TSLP-R antibody, or antigen-binding fragment thereof, described herein is indicated for treatment of CRSwNP in a subject that has never previously received a treatment or surgery for nasal polyposis. In some embodiments, the anti-TSLP-R antibody, or antigen-binding fragment thereof, described herein is indicated for treatment of CRSwNP in a subject that has a relapse of nasal polyposis after receiving surgery to treat the polyps. In some embodiments, the anti-TSLP-R antibody, or antigen-binding fragment thereof, described herein is indicated for the long-term treatment of adults with CRSwNP, who have persistent signs and symptoms despite treatment with intranasal corticosteroids (INCS). In some embodiments, the anti-TSLP-R antibody, or antigen-binding fragment thereof, described herein is indicated for the long-term treatment of adults with CRSwNP who have persistent signs and symptoms despite treatment with INCS.

In some embodiments, the method of treating CRSwNP comprises administering to a subject a therapeutically effective amount of an anti-TSLP-R antibody, or antigen-binding fragment thereof, as provided for herein.

In some embodiments, the method of treating CRSwNP in a subject in need thereof comprises administering to the subject a therapeutically effective amount of the anti-TSLP-R antibody, or antigen-binding fragment thereof, wherein the subject in need thereof is a human or non-human animal that exhibits one or more symptoms or indications of nasal polyposis, chronic sinusitis with nasal polyps, allergic or non-allergic rhinitis, or allergic or non-allergic rhinosinusitis. In some embodiments, the method of treating CRSwNP in a subject in need thereof comprises administering to the subject a therapeutically effective amount of the anti-TSLP-R antibody, or antigen-binding fragment thereof, wherein the subject in need thereof has bilateral nasal polyps, and a nasal polyp score of at least 4 out of a maximum of 8 for both nostrils, with at least a score of 2 for each nostril. In some embodiments, the method of treating CRSwNP in a subject in need thereof comprises administering to the subject a therapeutically effective amount of the anti-TSLP-R antibody, or antigen-binding fragment thereof, wherein the subject in need thereof has polyps in the middle meatus. In some embodiments, the method of treating CRSwNP in a subject in need thereof comprises administering to the subject a therapeutically effective amount of the anti-TSLP-R antibody, or antigen-binding fragment thereof, wherein the subject in need thereof has nasal polyps as confirmed by endoscopy. In some embodiments, the method of treating CRSwNP in a subject in need thereof comprises administering to the subject a therapeutically effective amount of the anti-TSLP-R antibody, or antigen-binding fragment thereof, wherein the subject in need thereof is has bilateral mucosal disease, which is confirmed by a method such as CT scan. As used herein “bilateral mucosal disease” is an infection of the mucous lining of the sinus cavities, e.g., the maxillary sinus cavities. In some embodiments, bilateral nasal polyposis (e.g., a nasal polyp score of at least 4 out of a maximum of 8 for both nostrils, with at least a score of 2 for each nostril) persists even after a treatment regimen of inhaled corticosteroids (INCS), such as where the INCS was administered for at least 6 weeks, at least 7 weeks, at least 8 weeks, or longer.

In some embodiments, the method of treating CRSwNP in a subject in need thereof comprises administering to the subject a therapeutically effective amount of the anti-TSLP-R antibody, or antigen-binding fragment thereof, wherein the subject in need thereof is has anterior and/or posterior mucopurulent drainage, nasal obstruction, and a decreased sense of smell. In some embodiments, the method of treating CRSwNP in a subject in need thereof comprises administering to the subject a therapeutically effective amount of the anti-TSLP-R antibody, or antigen-binding fragment thereof, wherein the subject in need thereof is has had symptoms of chronic sinusitis with nasal polyps for 6 weeks, 7 weeks, 8 weeks, 9 weeks, 10 weeks, 11 weeks, 12 weeks or more, prior to treatment with the anti-TSLP-R antibody, or antigen-binding fragment thereof. In some embodiments, the method of treating CRSwNP in a subject in need thereof comprises administering to the subject a therapeutically effective amount of the anti-TSLP-R antibody, or antigen-binding fragment thereof, wherein the subject in need thereof has received a previous treatment, such as with an intranasal corticosteroid (e.g., MFNS), for at least 4 weeks, at least 5 weeks, at least 6 weeks, at least 7 weeks, at least 8 weeks, at least 9 weeks, at least 10 weeks or longer, prior to receiving treatment with the anti-TSLP-R antibody, or antigen-binding fragment thereof. In some embodiments the subject will continue to receive the INCS while receiving treatment with the anti-TSLP-R antibody, or antigen-binding fragment thereof. In some embodiments, the subject stops receiving the INCS before receiving treatment with the anti-TSLP-R antibody, or antigen-binding fragment thereof, or the subject stops receiving treatment with the INCS if administration with the anti-TSLP-R antibody, or antigen-binding fragment thereof is effective to treat the chronic sinusitis with nasal polyps. In some embodiments, the subject tapers the dose of the INCS before stopping treatment completely.

In some embodiments, the method of treating CRSwNP in a subject in need thereof comprises administering to the subject a therapeutically effective amount of the anti-TSLP-R antibody, or antigen-binding fragment thereof, wherein the subject in need thereof is diagnosed with nasal polyposis or a condition associated with nasal polyposis, such as chronic sinusitis with nasal polyps, allergic or non-allergic rhinitis, or allergic or non-allergic rhinosinusitis. In some embodiments, the method of treating CRSwNP in a subject in need thereof comprises administering to the subject a therapeutically effective amount of the anti-TSLP-R antibody, or antigen-binding fragment thereof, wherein the subject in need thereof is diagnosed with nasal polyposis or a condition associated with nasal polyposis, such as chronic sinusitis with nasal polyps, allergic or non-allergic rhinitis, or allergic or non-allergic rhinosinusitis on the basis of one or more of the following: 22-item Sino Nasal Outcome Test (SNOT-22) score; subject-assessed nasal congestion/obstruction, anterior rhinorrhea, posterior rhinorrhea and loss of sense of smell; number of nocturnal awakenings; Visual Analog Score (VAS) to assess patient-rated rhinosinusitis symptom severity; five-item Asthma Control Questionnaire (ACQ5) score in patients with asthma; Nasal Polyp Score (NPS); Nasal Congestion Score (NCS); Peak Inspiratory Flow (NPIF); smell test (University of Pennsylvania Smell Identification Test (UPSIT); spirometry; physiological parameters, such as measured by nasal endoscopy and CT scan; Lund-Mackay Score; Three Dimensional volumetric measurement of the maxillary sinus, and patient reported outcomes.

In some embodiments, the method of treating CRSwNP in a subject in need thereof comprises administering to the subject a therapeutically effective amount of the anti-TSLP-R antibody, or antigen-binding fragment thereof, wherein the subject in need thereof is a human patient with chronic symptoms of sinusitis, which are the presence of at least two of the following symptoms: nasal blockade/obstruction/congestion or nasal discharge (anterior/posterior nasal drip); facial pain/pressure; and reduction or loss of smell.

In some embodiments, the method of treating CRSwNP in a subject in need thereof comprises administering to the subject a therapeutically effective amount of the anti-TSLP-R antibody, or antigen-binding fragment thereof, wherein the subject in need thereof is a human patient with a SNOT-22 score of greater than about 7, greater than about 10, greater than about 15, greater than about 20, greater than about 25, greater than about 30, greater than about 35, greater than about 40, greater than about 45, or greater than about 50. A “subject in need thereof” may also be a human patient who exhibits a Lund-Mackay score of greater than about 4, greater than about 5, greater than about 6, greater than about 7, greater than about 8, greater than about 9, greater than about 10, greater than about 11, greater than about 12, or greater than about 13.

In some embodiments, the method of treating CRSwNP in a subject in need thereof comprises administering to the subject a therapeutically effective amount of the anti-TSLP-R antibody, or antigen-binding fragment thereof, wherein the subject in need thereof a subject who, prior to receiving the anti-TSLP-R antibody, or antigen-binding fragment thereof, has been prescribed or is currently taking another medication, “a background therapy.” The background therapy can be, for example, an intranasal corticosteroid (INCS, or ICS), such as Mometasone furoate nasal spray (MFNS; Nasonex®). In some embodiments, the method of treating CRSwNP in a subject in need thereof comprises administering to the subject a therapeutically effective amount of the anti-TSLP-R antibody, or antigen-binding fragment thereof, wherein the subject in need thereof is an asthma patient who prior to receiving the anti-TSLP-R antibody, or antigen-binding fragment thereof, has been prescribed or is currently taking an INCS in combination with a long-acting beta-2-adrenergic antagonist (LABA). Examples of INCS/LABA therapies include fluticasone/salmeterol combination therapy and budesonide/formoterol combination therapy. In some embodiments, the background therapy is a nasal saline, a topical decongestant, a topical anesthetic, a leukotriene antagonist or a systemic antihistamine. In some embodiments, the “subject in need thereof” continues the background therapy after the subject receives the anti-TSLP-R antibody, or antigen-binding fragment thereof, and in other embodiments, the subject in need thereof stops receiving the background therapy (e.g., at once or gradually) before receiving the anti-TSLP-R antibody, or antigen-binding fragment thereof.

In some embodiments, the method of treating CRSwNP comprises administering an anti-TSLP-R antibody, or antigen-binding fragment thereof, described herein to a subject to treat, inhibit or ameliorate Chronic Rhinosinusitis with Nasal Polyps. In some embodiments, the method of treating CRSwNP comprises administering a therapeutically effective amount of the anti-TSLP-R antibody, or antigen-binding fragment thereof, as disclosed herein, or the pharmaceutical dosage form of the anti-TSLP-R antibody, or antigen-binding fragment thereof, as disclosed herein, to a subject in need thereof, wherein the treatment results in an improvement of or reduction in severity of a symptom of the disease. In some embodiments, the method of treating CRSwNP comprises delaying the onset of Chronic Rhinosinusitis with Nasal Polyps in a subject in need thereof. In some embodiments, the method of treating CRSwNP comprises preventing of Chronic Rhinosinusitis with Nasal Polyps in a subject in need thereof. In some embodiments, the method of treating CRSwNP comprises increasing the internalization of TSLP-R on a cell. In some embodiments, the method of treating CRSwNP comprises inhibiting TSLP-R on a cell. In some embodiments, the method of treating CRSwNP comprises inhibiting TSLP-R by at least 95%, 96%, 97%, 98%, 99%, or by 100% in a subject in need thereof.

In some embodiments, the method of treating CRSwNP comprises administering to the subject a therapeutically effective amount of an anti-TSLP-R antibody, or antigen-binding fragment thereof, comprising: (i) a heavy chain variable region comprising heavy chain CDR1, CDR2, and CDR3 sequences, wherein the heavy chain CDR1 sequence has the amino acid sequence of SEQ ID NO: 7; the heavy chain CDR2 has the amino acid sequence of SEQ ID NO: 8; and the heavy chain CDR3 sequence has the amino acid sequence of SEQ ID NO: 9; or variants of any of the foregoing; and (ii) a light chain variable region comprising light chain CDR1, CDR2, and CDR3 sequences, wherein the light chain CDR1 sequence has the amino acid sequence SEQ ID NO: 10; the light chain CDR2 sequence has the amino acid sequence of SEQ ID NO: 11; and the light chain CDR3 sequence has the amino acid sequence of SEQ ID NO: 12; or variants of any of the foregoing.

In some embodiments, the method of treating CRSwNP comprises administering to a subject a therapeutically effective amount of an anti-TSLP-R antibody, or antigen-binding fragment thereof, comprising a heavy chain variable region having a sequence that is at least 90%, 91%, 92%, 93%, 94%, 95%, 96%, 97%, 98%, or 99% identical to the sequence of SEQ ID NO: 5. In some embodiments, the method of treating CRSwNP comprises administering to a subject a therapeutically effective amount of an anti-TSLP-R antibody, or antigen-binding fragment thereof, comprising a heavy chain variable region having a sequence that is at least 90%, 91%, 92%, 93%, 94%, 95%, 96%, 97%, 98%, or 99% identical to the sequence of SEQ ID NO: 5, provided that the CDRs comprises the amino acid sequences of SEQ ID NO: 7, SEQ ID NO: 8, and SEQ ID NO: 9. In some embodiments, the method of treating CRSwNP comprises administering to a subject a therapeutically effective amount of an anti-TSLP-R antibody, or antigen-binding fragment thereof, comprising a heavy chain variable region having a sequence that is at least 90%, 91%, 92%, 93%, 94%, 95%, 96%, 97%, 98%, or 99% identical to the sequence of SEQ ID NO: 5, provided that the CDRs comprise the HCDR1 amino acid sequence of SEQ ID NO: 7; the HCDR2 amino acid sequence of SEQ ID NO: 8, and the HCDR3 amino acid sequence of SEQ ID NO: 9.

In some embodiments, the method of treating CRSwNP comprises administering to a subject a therapeutically effective amount of an anti-TSLP-R antibody, or antigen-binding fragment thereof, comprising a light chain variable region having a sequence that is at least 90%, 91%, 92%, 93%, 94%, 95%, 96%, 97%, 98%, or 99% identical to the sequence of SEQ ID NO: 6. In some embodiments, the method of treating CRSwNP comprises administering to a subject a therapeutically effective amount of an anti-TSLP-R antibody, or antigen-binding fragment thereof, comprising a light chain variable region having a sequence that is at least 90%, 91%, 92%, 93%, 94%, 95%, 96%, 97%, 98%, or 99% identical to the sequence of SEQ ID NO: 6, provided that the light chain variable region comprises the amino acid sequences of SEQ ID NO: 10, SEQ ID NO: 11, and SEQ ID NO: 12. In some embodiments, the method of treating CRSwNP comprises administering to a subject a therapeutically effective amount of an anti-TSLP-R antibody, or antigen-binding fragment thereof, comprising a light chain variable region having a sequence that is at least 90%, 91%, 92%, 93%, 94%, 95%, 96%, 97%, 98%, or 99% identical to the sequence of SEQ ID NO: 6, provided that the CDRs comprise the LCDR1 amino acid sequence of SEQ ID NO: 10; the LCDR2 amino acid sequence of SEQ ID NO: 11, and the LCDR3 amino acid sequence of SEQ ID NO: 12.

In some embodiments, the method of treating CRSwNP comprises administering to a subject a therapeutically effective amount of an anti-TSLP-R antibody, or antigen-binding fragment thereof, comprising: (i) a heavy chain variable region having a sequence that is at least 90%, 91%, 92%, 93%, 94%, 95%, 96%, 97%, 98%, or 99% identical to the sequence of SEQ ID NO: 5; and (ii) a light chain variable region having a sequence that is at least 90%, 91%, 92%, 93%, 94%, 95%, 96%, 97%, 98%, or 99% identical to the sequence of SEQ ID NO: 6.

In some embodiments, the method of treating CRSwNP comprises administering to a subject a therapeutically effective amount of an anti-TSLP-R antibody, or antigen-binding fragment thereof, comprising: (i) a heavy chain variable region having a sequence that is at least 90%, 91%, 92%, 93%, 94%, 95%, 96%, 97%, 98%, or 99% identical to the sequence of SEQ ID NO: 5; and (ii) a light chain variable region having a sequence that is at least 90%, 91%, 92%, 93%, 94%, 95%, 96%, 97%, 98%, or 99% identical to the sequence of SEQ ID NO: 6. In some embodiments, the method of treating CRSwNP comprises administering to a subject a therapeutically effective amount of an anti-TSLP-R antibody, or antigen-binding fragment thereof, comprising: (i) a heavy chain variable region having a sequence that is at least 90%, 91%, 92%, 93%, 94%, 95%, 96%, 97%, 98%, or 99% identical to the sequence of SEQ ID NO: 5, provided that the CDRs comprise the HCDR1 amino acid sequence of SEQ ID NO: 7; the HCDR2 amino acid sequence of SEQ ID NO: 8, and the HCDR3 amino acid sequence of SEQ ID NO: 9; and (ii) a light chain variable region having a sequence that is at least 90%, 91%, 92%, 93%, 94%, 95%, 96%, 97%, 98%, or 99% identical to the sequence of SEQ ID NO: 6, provided that the CDRs comprise the LCDR1 amino acid sequence of SEQ ID NO: 10; the LCDR2 amino acid sequence of SEQ ID NO: 11, and the LCDR3 amino acid sequence of SEQ ID NO: 12.

In some embodiments, the method of treating CRSwNP comprises administering to a subject a therapeutically effective amount of an anti-TSLP-R antibody, or antigen-binding fragment thereof, comprising a heavy chain variable region having a sequence of SEQ ID NO: 5. In some embodiments, the method of treating CRSwNP comprises administering to a subject a therapeutically effective amount of an anti-TSLP-R antibody, or antigen-binding fragment thereof, comprising a light chain variable region having a sequence of SEQ ID NO: 6. In some embodiments, the method of treating CRSwNP comprises administering to a subject a therapeutically effective amount of an anti-TSLP-R antibody, or antigen-binding fragment thereof, comprising: (i) a heavy chain variable region having a sequence of SEQ ID NO: 5; and (ii) a light chain variable region having a sequence of SEQ ID NO: 6.

In some embodiments, the method of treating CRSwNP comprises administering to the subject a therapeutically effective amount of the anti-TSLP-R antibody, or antigen-binding fragment thereof, comprising a heavy chain having a sequence that is at least 90%, 91%, 92%, 93%, 94%, 95%, 96%, 97%, 98%, or 99% identical to the sequence of SEQ ID NO: 1. In some embodiments, the method of treating CRSwNP comprises administering to the subject a therapeutically effective amount of the anti-TSLP-R antibody, or antigen-binding fragment thereof, comprising a light chain having a sequence that is at least 90%, 91%, 92%, 93%, 94%, 95%, 96%, 97%, 98%, or 99% identical to the sequence of SEQ ID NO: 3. In some embodiments, the method of treating CRSwNP comprises administering to the subject a therapeutically effective amount of the anti-TSLP-R antibody, or antigen-binding fragment thereof, comprising a heavy chain having a sequence of SEQ ID NO: 1. In some embodiments, the method of treating CRSwNP comprises administering to the subject a therapeutically effective amount of the anti-TSLP-R antibody, or antigen-binding fragment thereof, comprising a light chain having a sequence of SEQ ID NO: 3.

In some embodiments, the method of treating CRSwNP comprises administering to the subject a therapeutically effective amount of the anti-TSLP-R antibody, or antigen-binding fragment thereof, comprising: (i) a heavy chain having a sequence that is at least 90%, 91%, 92%, 93%, 94%, 95%, 96%, 97%, 98%, or 99% identical to the sequence of SEQ ID NO: 1; and (ii) a light chain having a sequence that is at least 90%, 91%, 92%, 93%, 94%, 95%, 96%, 97%, 98%, or 99% identical to the sequence of SEQ ID NO: 3. In some embodiments, the method of treating CRSwNP comprises administering to the subject a therapeutically effective amount of the anti-TSLP-R antibody, or antigen-binding fragment thereof, comprising: (i) a heavy chain having a sequence of SEQ ID NO: 1; and (ii) a light chain having a sequence of SEQ ID NO: 3.

In some embodiments, the method of treating CRSwNP comprises administering to the subject a therapeutically effective amount of the anti-TSLP-R antibody, or antigen-binding fragment thereof, at about 1.0 mg/kg to about 15.0 mg/kg. In some embodiments, the method of treating CRSwNP comprises administering to the subject a therapeutically effective amount of the anti-TSLP-R antibody, or antigen-binding fragment thereof, as an initial dose, then a maintenance dose.

In some embodiments, the method of treating CRSwNP comprises administering to the subject a therapeutically effective amount of the anti-TSLP-R antibody, or antigen-binding fragment thereof, as an initial dose of about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg, about 5.1 mg/kg, about 5.2 mg/kg, about 5.3 mg/kg, about 5.4 mg/kg, about 5.5 mg/kg, about 5.6 mg/kg, about 5.7 mg/kg, about 5.8 mg/kg, about 5.9 mg/kg, about 6.0 mg/kg, about 6.1 mg/kg, about 6.2 mg/kg, about 6.3 mg/kg, about 6.4 mg/kg, about 6.5 mg/kg, about 6.6 mg/kg, about 6.7 mg/kg, about 6.8 mg/kg, about 6.9 mg/kg, about 7.0 mg/kg, about 7.1 mg/kg, about 7.2 mg/kg, about 7.3 mg/kg, about 7.4 mg/kg, about 7.5 mg/kg, about 7.6 mg/kg, about 7.7 mg/kg, about 7.8 mg/kg, about 7.9 mg/kg, about 8.0 mg/kg, about 8.1 mg/kg, about 8.2 mg/kg, about 8.3 mg/kg, about 8.4 mg/kg, about 8.5 mg/kg, about 8.6 mg/kg, about 8.7 mg/kg, about 8.8 mg/kg, about 8.9 mg/kg, about 9.0 mg/kg, about 9.1 mg/kg, about 9.2 mg/kg, about 9.3 mg/kg, about 9.4 mg/kg, about 9.5 mg/kg, about 9.6 mg/kg, about 9.7 mg/kg, about 9.8 mg/kg, about 9.9 mg/kg, about 10.0 mg/kg, about 10.1 mg/kg, about 10.2 mg/kg, about 10.3 mg/kg, about 10.4 mg/kg, about 10.5 mg/kg, about 10.6 mg/kg, about 10.7 mg/kg, about 10.8 mg/kg, about 10.9 mg/kg, about 11.0 mg/kg, about 11.1 mg/kg, about 11.2 mg/kg, about 11.3 mg/kg, about 11.4 mg/kg, about 11.5 mg/kg, about 11.6 mg/kg, about 11.7 mg/kg, about 11.8 mg/kg, about 11.9 mg/kg, about 12.0 mg/kg, about 12.1 mg/kg, about 12.2 mg/kg, about 12.3 mg/kg, about 12.4 mg/kg, about 12.5 mg/kg, about 12.6 mg/kg, about 12.7 mg/kg, about 12.8 mg/kg, about 12.9 mg/kg, about 13.0 mg/kg, about 13.1 mg/kg, about 13.2 mg/kg, about 13.3 mg/kg, about 13.4 mg/kg, about 13.5 mg/kg, about 13.6 mg/kg, about 13.7 mg/kg, about 13.8 mg/kg, about 13.9 mg/kg, about 14.0 mg/kg, about 14.1 mg/kg, about 14.2 mg/kg, about 14.3 mg/kg, about 14.4 mg/kg, about 14.5 mg/kg, about 14.6 mg/kg, about 14.7 mg/kg, about 14.8 mg/kg, about 14.9 mg/kg, or about 15.0 mg/kg.

In some embodiments, the method of treating CRSwNP comprises administering to the subject a therapeutically effective amount of the anti-TSLP-R antibody, or antigen-binding fragment thereof, as a maintenance dose or doses of about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg, about 5.1 mg/kg, about 5.2 mg/kg, about 5.3 mg/kg, about 5.4 mg/kg, about 5.5 mg/kg, about 5.6 mg/kg, about 5.7 mg/kg, about 5.8 mg/kg, about 5.9 mg/kg, about 6.0 mg/kg, about 6.1 mg/kg, about 6.2 mg/kg, about 6.3 mg/kg, about 6.4 mg/kg, about 6.5 mg/kg, about 6.6 mg/kg, about 6.7 mg/kg, about 6.8 mg/kg, about 6.9 mg/kg, about 7.0 mg/kg, about 7.1 mg/kg, about 7.2 mg/kg, about 7.3 mg/kg, about 7.4 mg/kg, about 7.5 mg/kg, about 7.6 mg/kg, about 7.7 mg/kg, about 7.8 mg/kg, about 7.9 mg/kg, about 8.0 mg/kg, about 8.1 mg/kg, about 8.2 mg/kg, about 8.3 mg/kg, about 8.4 mg/kg, about 8.5 mg/kg, about 8.6 mg/kg, about 8.7 mg/kg, about 8.8 mg/kg, about 8.9 mg/kg, about 9.0 mg/kg, about 9.1 mg/kg, about 9.2 mg/kg, about 9.3 mg/kg, about 9.4 mg/kg, about 9.5 mg/kg, about 9.6 mg/kg, about 9.7 mg/kg, about 9.8 mg/kg, about 9.9 mg/kg, about 10.0 mg/kg, about 10.1 mg/kg, about 10.2 mg/kg, about 10.3 mg/kg, about 10.4 mg/kg, about 10.5 mg/kg, about 10.6 mg/kg, about 10.7 mg/kg, about 10.8 mg/kg, about 10.9 mg/kg, about 11.0 mg/kg, about 11.1 mg/kg, about 11.2 mg/kg, about 11.3 mg/kg, about 11.4 mg/kg, about 11.5 mg/kg, about 11.6 mg/kg, about 11.7 mg/kg, about 11.8 mg/kg, about 11.9 mg/kg, about 12.0 mg/kg, about 12.1 mg/kg, about 12.2 mg/kg, about 12.3 mg/kg, about 12.4 mg/kg, about 12.5 mg/kg, about 12.6 mg/kg, about 12.7 mg/kg, about 12.8 mg/kg, about 12.9 mg/kg, about 13.0 mg/kg, about 13.1 mg/kg, about 13.2 mg/kg, about 13.3 mg/kg, about 13.4 mg/kg, about 13.5 mg/kg, about 13.6 mg/kg, about 13.7 mg/kg, about 13.8 mg/kg, about 13.9 mg/kg, about 14.0 mg/kg, about 14.1 mg/kg, about 14.2 mg/kg, about 14.3 mg/kg, about 14.4 mg/kg, about 14.5 mg/kg, about 14.6 mg/kg, about 14.7 mg/kg, about 14.8 mg/kg, about 14.9 mg/kg, or about 15.0 mg/kg.

In some embodiments, the method of treating CRSwNP comprises administering to the subject a therapeutically effective amount of the anti-TSLP-R antibody, or antigen-binding fragment thereof, as an initial dose of about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg.

In some embodiments, the method of treating CRSwNP comprises administering to the subject a therapeutically effective amount of the anti-TSLP-R antibody, or antigen-binding fragment thereof, as a maintenance dose or doses of about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg.

In some embodiments, the method of treating CRSwNP comprises administering to the subject a therapeutically effective amount of the anti-TSLP-R antibody, or antigen-binding fragment thereof, at an initial dose of about 25 mg, 50 mg, 100 mg, 150 mg, 200 mg, 250 mg, 300 mg, 350 mg, 400 mg, 500 mg, 600 mg, 700 mg, or 800 mg of the antibody. In some embodiments, the method of treating CRSwNP comprises administering to the subject a therapeutically effective amount of the anti-TSLP-R antibody, or antigen-binding fragment thereof, at an initial dose of about 25 mg of the antibody. In some embodiments, the method of treating CRSwNP comprises administering to the subject a therapeutically effective amount of the anti-TSLP-R antibody, or antigen-binding fragment thereof, at an initial dose of about 50 mg of the antibody. In some embodiments, the method of treating CRSwNP comprises administering to the subject a therapeutically effective amount of the anti-TSLP-R antibody, or antigen-binding fragment thereof, at an initial dose of about 100 mg of the antibody. In some embodiments, the method of treating CRSwNP comprises administering to the subject a therapeutically effective amount of the anti-TSLP-R antibody, or antigen-binding fragment thereof, at an initial dose of about 150 mg of the antibody. In some embodiments, the method of treating CRSwNP comprises administering to the subject a therapeutically effective amount of the anti-TSLP-R antibody, or antigen-binding fragment thereof, at an initial dose of about 200 mg of the antibody. In some embodiments, the method of treating CRSwNP comprises administering to the subject a therapeutically effective amount of the anti-TSLP-R antibody, or antigen-binding fragment thereof, at an initial dose of about 250 mg of the antibody. In some embodiments, the method of treating CRSwNP comprises administering to the subject a therapeutically effective amount of the anti-TSLP-R antibody, or antigen-binding fragment thereof, at an initial dose of about 300 mg of the antibody. In some embodiments, the method of treating CRSwNP comprises administering to the subject a therapeutically effective amount of the anti-TSLP-R antibody, or antigen-binding fragment thereof, at an initial dose of about 400 mg of the antibody. In some embodiments, the method of treating CRSwNP comprises administering to the subject a therapeutically effective amount of the anti-TSLP-R antibody, or antigen-binding fragment thereof, at an initial dose of about 500 mg of the antibody. In some embodiments, the method of treating CRSwNP comprises administering to the subject a therapeutically effective amount of the anti-TSLP-R antibody, or antigen-binding fragment thereof, at an initial dose of about 600 mg of the antibody. In some embodiments, the method of treating CRSwNP comprises administering to the subject a therapeutically effective amount of the anti-TSLP-R antibody, or antigen-binding fragment thereof, at an initial dose of about 700 mg of the antibody. In some embodiments, the method of treating CRSwNP comprises administering to the subject a therapeutically effective amount of the anti-TSLP-R antibody, or antigen-binding fragment thereof, at an initial dose of about 800 mg of the antibody.

In some embodiments, the method of treating CRSwNP comprises administering to the subject a therapeutically effective amount of the anti-TSLP-R antibody, or antigen-binding fragment thereof, at a maintenance dose or doses of about 25 mg, 50 mg, 100 mg, 150 mg, 200 mg, 250 mg, 300 mg, 350 mg, 400 mg, 500 mg, 600 mg, 700 mg, or 800 mg of the antibody. In some embodiments, the method of treating CRSwNP comprises administering to the subject a therapeutically effective amount of the anti-TSLP-R antibody, or antigen-binding fragment thereof, in a maintenance dose or doses of about 25 mg of the antibody. In some embodiments, the method of treating CRSwNP comprises administering to the subject a therapeutically effective amount of the anti-TSLP-R antibody, or antigen-binding fragment thereof, in a maintenance dose or doses of about 50 mg of the antibody. In some embodiments, the method of treating CRSwNP comprises administering to the subject a therapeutically effective amount of the anti-TSLP-R antibody, or antigen-binding fragment thereof, in a maintenance dose or doses of about 100 mg of the antibody. In some embodiments, the method of treating CRSwNP comprises administering to the subject a therapeutically effective amount of the anti-TSLP-R antibody, or antigen-binding fragment thereof, in a maintenance dose or doses of about 150 mg of the antibody. In some embodiments, the method of treating CRSwNP comprises administering to the subject a therapeutically effective amount of the anti-TSLP-R antibody, or antigen-binding fragment thereof, in a maintenance dose or doses of about 200 mg of the antibody. In some embodiments, the method of treating CRSwNP comprises administering to the subject a therapeutically effective amount of the anti-TSLP-R antibody, or antigen-binding fragment thereof, in a maintenance dose or doses of about 250 mg of the antibody. In some embodiments, the method of treating CRSwNP comprises administering to the subject a therapeutically effective amount of the anti-TSLP-R antibody, or antigen-binding fragment thereof, in a maintenance dose or doses of about 300 mg of the antibody. In some embodiments, the method of treating CRSwNP comprises administering to the subject a therapeutically effective amount of the anti-TSLP-R antibody, or antigen-binding fragment thereof, in a maintenance dose or doses of about 400 mg of the antibody. In some embodiments, the method of treating CRSwNP comprises administering to the subject a therapeutically effective amount of the anti-TSLP-R antibody, or antigen-binding fragment thereof, in a maintenance dose or doses of about 500 mg of the antibody. In some embodiments, the method of treating CRSwNP comprises administering to the subject a therapeutically effective amount of the anti-TSLP-R antibody, or antigen-binding fragment thereof, in a maintenance dose or doses of about 600 mg of the antibody. In some embodiments, the method of treating CRSwNP comprises administering to the subject a therapeutically effective amount of the anti-TSLP-R antibody, or antigen-binding fragment thereof, in a maintenance dose or doses of about 700 mg of the antibody. In some embodiments, the method of treating CRSwNP comprises administering to the subject a therapeutically effective amount of the anti-TSLP-R antibody, or antigen-binding fragment thereof, in a maintenance dose or doses of about 800 mg of the antibody.

In some embodiments, the method of treating CRSwNP comprises administering to the subject a therapeutically effective amount of the anti-TSLP-R antibody, or antigen-binding fragment thereof, by injection. In some embodiments, the injection is an intraarterial injection, an intraarticular injection, an intracameral injection, an intracerebral injection, an intradermal injection, an intramedullary injection, an intramuscular injection, an intraocular/intravitreal injection, an intraperitoneal injection, an intrathecal/intraspinal injection, an intravenous injection, an intraventricular injection, a subcutaneous injection, a suprachoroidal injection, or a retroorbital injection. In some embodiments, the method of treating CRSwNP comprises administering to the subject a therapeutically effective amount of the anti-TSLP-R antibody, or antigen-binding fragment thereof, subcutaneously. In some embodiments, the method of treating Chronic Rhinosinusitis with Nasal Polyps comprises administering to the subject a therapeutically effective amount of the anti-TSLP-R antibody, or antigen-binding fragment thereof, by subcutaneous injection. In some embodiments, the method of treating CRSwNP comprises administering to the subject a therapeutically effective amount of the anti-TSLP-R antibody, or antigen-binding fragment thereof, intravenously. In some embodiments, the method of treating CRSwNP comprises administering to the subject a therapeutically effective amount of the anti-TSLP-R antibody, or antigen-binding fragment thereof, by intravenous injection.

In some embodiments, a method of treating Chronic Rhinosinusitis with Nasal Polyps in a subject in need thereof, comprises administering to the subject a therapeutically effective amount of an anti-TSLP-R antibody, or antigen-binding fragment thereof, at a concentration of about 1 mg/mL to about 300 mg/mL. In some embodiments, a method of treating Chronic Rhinosinusitis with Nasal Polyps in a subject in need thereof, comprises administering to the subject a therapeutically effective amount of an anti-TSLP-R antibody, or antigen-binding fragment thereof, at a concentration of about 300 mg/mL. In some embodiments, a method of treating Chronic Rhinosinusitis with Nasal Polyps in a subject in need thereof, comprises administering to the subject a therapeutically effective amount of an anti-TSLP-R antibody, or antigen-binding fragment thereof, at a concentration of about 1 mg/mL to about 200 mg/mL. In some embodiments, a method of treating Chronic Rhinosinusitis with Nasal Polyps in a subject in need thereof, comprises administering to the subject a therapeutically effective amount of an anti-TSLP-R antibody, or antigen-binding fragment thereof, at a concentration of about 1 mg/mL to about 150 mg/mL.

In some embodiments, the method of treating CRSwNP comprises administering to the subject a therapeutically effective amount of the anti-TSLP-R antibody, or antigen-binding fragment thereof, at least once every 1 day, once every 1 week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, once every 26 weeks, once every 27 weeks, once every 28 weeks, once every 29 weeks, once every 30 weeks, once every 31 weeks, once every 32 weeks, once every 33 weeks, once every 34 weeks, once every 35 weeks, once every 36 weeks, once every 37 weeks, once every 38 weeks, once every 39 weeks, once every 40 weeks, once every 41 weeks, once every 42 weeks, once every 43 weeks, once every 44 weeks, once every 45 weeks, once every 46 weeks, once every 47 weeks, once every 48 weeks, once every 49 weeks, once every 50 weeks, once every 51 weeks, once every 52 weeks, once every 1 month, once every 2 months, once every 3 months, once every 4 months, once every 5 months, once every 6 months, once every 7 months, once every 8 months, once every 9 months, once every 10 months, once every 11 months, or once every 12 months.

In some embodiments, the method of treating CRSwNP comprises administering to the subject a therapeutically effective amount of the anti-TSLP-R antibody, or antigen-binding fragment thereof, at least once every 1 day. In some embodiments, the method of treating CRSwNP comprises administering to the subject a therapeutically effective amount of the anti-TSLP-R antibody, or antigen-binding fragment thereof, at least once every 1 week. In some embodiments, the method of treating CRSwNP comprises administering to the subject a therapeutically effective amount of the anti-TSLP-R antibody, or antigen-binding fragment thereof, at least once every 2 weeks. In some embodiments, the method of treating CRSwNP comprises administering to the subject a therapeutically effective amount of the anti-TSLP-R antibody, or antigen-binding fragment thereof, at least once every 3 weeks. In some embodiments, the method of treating CRSwNP comprises administering to the subject a therapeutically effective amount of the anti-TSLP-R antibody, or antigen-binding fragment thereof, at least once every 4 weeks. In some embodiments, the method of treating CRSwNP comprises administering to the subject a therapeutically effective amount of the anti-TSLP-R antibody, or antigen-binding fragment thereof, at least once every 5 weeks. In some embodiments, the method of treating CRSwNP comprises administering to the subject a therapeutically effective amount of the anti-TSLP-R antibody, or antigen-binding fragment thereof, at least once every 6 weeks. In some embodiments, the method of treating CRSwNP comprises administering to the subject a therapeutically effective amount of the anti-TSLP-R antibody, or antigen-binding fragment thereof, at least once every 7 weeks. In some embodiments, the method of treating CRSwNP comprises administering to the subject a therapeutically effective amount of the anti-TSLP-R antibody, or antigen-binding fragment thereof, at least once every 8 weeks. In some embodiments, the method of treating CRSwNP comprises administering to the subject a therapeutically effective amount of the anti-TSLP-R antibody, or antigen-binding fragment thereof, at least once every 9 weeks. In some embodiments, the method of treating CRSwNP comprises administering to the subject a therapeutically effective amount of the anti-TSLP-R antibody, or antigen-binding fragment thereof, at least once every 10 weeks. In some embodiments, the method of treating CRSwNP comprises administering to the subject a therapeutically effective amount of the anti-TSLP-R antibody, or antigen-binding fragment thereof, at least once every 11 weeks. In some embodiments, the method of treating CRSwNP comprises administering to the subject a therapeutically effective amount of the anti-TSLP-R antibody, or antigen-binding fragment thereof, at least once every 12 weeks. In some embodiments, the method of treating CRSwNP comprises administering to the subject a therapeutically effective amount of the anti-TSLP-R antibody, or antigen-binding fragment thereof, at least once every 13 weeks. In some embodiments, the method of treating CRSwNP comprises administering to the subject a therapeutically effective amount of the anti-TSLP-R antibody, or antigen-binding fragment thereof, at least once every 14 weeks. In some embodiments, the method of treating CRSwNP comprises administering to the subject a therapeutically effective amount of the anti-TSLP-R antibody, or antigen-binding fragment thereof, at least once every 15 weeks. In some embodiments, the method of treating CRSwNP comprises administering to the subject a therapeutically effective amount of the anti-TSLP-R antibody, or antigen-binding fragment thereof, at least once every 16 weeks. In some embodiments, the method of treating CRSwNP comprises administering to the subject a therapeutically effective amount of the anti-TSLP-R antibody, or antigen-binding fragment thereof, at least once every 17 weeks. In some embodiments, the method of treating CRSwNP comprises administering to the subject a therapeutically effective amount of the anti-TSLP-R antibody, or antigen-binding fragment thereof, at least once every 18 weeks. In some embodiments, the method of treating CRSwNP comprises administering to the subject a therapeutically effective amount of the anti-TSLP-R antibody, or antigen-binding fragment thereof, at least once every 19 weeks. In some embodiments, the method of treating CRSwNP comprises administering to the subject a therapeutically effective amount of the anti-TSLP-R antibody, or antigen-binding fragment thereof, at least once every 20 weeks. In some embodiments, the method of treating CRSwNP comprises administering to the subject a therapeutically effective amount of the anti-TSLP-R antibody, or antigen-binding fragment thereof, at least once every 21 weeks. In some embodiments, the method of treating CRSwNP comprises administering to the subject a therapeutically effective amount of the anti-TSLP-R antibody, or antigen-binding fragment thereof, at least once every 22 weeks. In some embodiments, the method of treating CRSwNP comprises administering to the subject a therapeutically effective amount of the anti-TSLP-R antibody, or antigen-binding fragment thereof, at least once every 23 weeks. In some embodiments, the method of treating CRSwNP comprises administering to the subject a therapeutically effective amount of the anti-TSLP-R antibody, or antigen-binding fragment thereof, at least once every 24 weeks. In some embodiments, the method of treating CRSwNP comprises administering to the subject a therapeutically effective amount of the anti-TSLP-R antibody, or antigen-binding fragment thereof, at least once every 25 weeks. In some embodiments, the method of treating CRSwNP comprises administering to the subject a therapeutically effective amount of the anti-TSLP-R antibody, or antigen-binding fragment thereof, at least once every 26 weeks. In some embodiments, the method of treating CRSwNP comprises administering to the subject a therapeutically effective amount of the anti-TSLP-R antibody, or antigen-binding fragment thereof, at least once every 27 weeks. In some embodiments, the method of treating CRSwNP comprises administering to the subject a therapeutically effective amount of the anti-TSLP-R antibody, or antigen-binding fragment thereof, at least once every 28 weeks. In some embodiments, the method of treating CRSwNP comprises administering to the subject a therapeutically effective amount of the anti-TSLP-R antibody, or antigen-binding fragment thereof, at least once every 29 weeks. In some embodiments, the method of treating CRSwNP comprises administering to the subject a therapeutically effective amount of the anti-TSLP-R antibody, or antigen-binding fragment thereof, at least once every 30 weeks. In some embodiments, the method of treating CRSwNP comprises administering to the subject a therapeutically effective amount of the anti-TSLP-R antibody, or antigen-binding fragment thereof, at least once every 31 weeks. In some embodiments, the method of treating CRSwNP comprises administering to the subject a therapeutically effective amount of the anti-TSLP-R antibody, or antigen-binding fragment thereof, at least once every 32 weeks. In some embodiments, the method of treating CRSwNP comprises administering to the subject a therapeutically effective amount of the anti-TSLP-R antibody, or antigen-binding fragment thereof, at least once every 33 weeks. In some embodiments, the method of treating CRSwNP comprises administering to the subject a therapeutically effective amount of the anti-TSLP-R antibody, or antigen-binding fragment thereof, at least once every 34 weeks. In some embodiments, the method of treating CRSwNP comprises administering to the subject a therapeutically effective amount of the anti-TSLP-R antibody, or antigen-binding fragment thereof, at least once every 35 weeks. In some embodiments, the method of treating CRSwNP comprises administering to the subject a therapeutically effective amount of the anti-TSLP-R antibody, or antigen-binding fragment thereof, at least once every 36 weeks. In some embodiments, the method of treating CRSwNP comprises administering to the subject a therapeutically effective amount of the anti-TSLP-R antibody, or antigen-binding fragment thereof, at least once every 37 weeks. In some embodiments, the method of treating CRSwNP comprises administering to the subject a therapeutically effective amount of the anti-TSLP-R antibody, or antigen-binding fragment thereof, at least once every 38 weeks. In some embodiments, the method of treating CRSwNP comprises administering to the subject a therapeutically effective amount of the anti-TSLP-R antibody, or antigen-binding fragment thereof, at least once every 39 weeks. In some embodiments, the method of treating CRSwNP comprises administering to the subject a therapeutically effective amount of the anti-TSLP-R antibody, or antigen-binding fragment thereof, at least once every 40 weeks. In some embodiments, the method of treating CRSwNP comprises administering to the subject a therapeutically effective amount of the anti-TSLP-R antibody, or antigen-binding fragment thereof, at least once every 41 weeks. In some embodiments, the method of treating CRSwNP comprises administering to the subject a therapeutically effective amount of the anti-TSLP-R antibody, or antigen-binding fragment thereof, at least once every 42 weeks. In some embodiments, the method of treating CRSwNP comprises administering to the subject a therapeutically effective amount of the anti-TSLP-R antibody, or antigen-binding fragment thereof, at least once every 43 weeks. In some embodiments, the method of treating CRSwNP comprises administering to the subject a therapeutically effective amount of the anti-TSLP-R antibody, or antigen-binding fragment thereof, at least once every 44 weeks. In some embodiments, the method of treating CRSwNP comprises administering to the subject a therapeutically effective amount of the anti-TSLP-R antibody, or antigen-binding fragment thereof, at least once every 45 weeks. In some embodiments, the method of treating CRSwNP comprises administering to the subject a therapeutically effective amount of the anti-TSLP-R antibody, or antigen-binding fragment thereof, at least once every 46 weeks. In some embodiments, the method of treating CRSwNP comprises administering to the subject a therapeutically effective amount of the anti-TSLP-R antibody, or antigen-binding fragment thereof, at least once every 47 weeks. In some embodiments, the method of treating CRSwNP comprises administering to the subject a therapeutically effective amount of the anti-TSLP-R antibody, or antigen-binding fragment thereof, at least once every 48 weeks. In some embodiments, the method of treating CRSwNP comprises administering to the subject a therapeutically effective amount of the anti-TSLP-R antibody, or antigen-binding fragment thereof, at least once every 49 weeks. In some embodiments, the method of treating CRSwNP comprises administering to the subject a therapeutically effective amount of the anti-TSLP-R antibody, or antigen-binding fragment thereof, at least once every 50 weeks. In some embodiments, the method of treating CRSwNP comprises administering to the subject a therapeutically effective amount of the anti-TSLP-R antibody, or antigen-binding fragment thereof, at least once every 51 weeks. In some embodiments, the method of treating CRSwNP comprises administering to the subject a therapeutically effective amount of the anti-TSLP-R antibody, or antigen-binding fragment thereof, at least once every 52 weeks. In some embodiments, the method of treating CRSwNP comprises administering to the subject a therapeutically effective amount of the anti-TSLP-R antibody, or antigen-binding fragment thereof, at least once every 1 month. In some embodiments, the method of treating CRSwNP comprises administering to the subject a therapeutically effective amount of the anti-TSLP-R antibody, or antigen-binding fragment thereof, at least once every 2 months. In some embodiments, the method of treating CRSwNP comprises administering to the subject a therapeutically effective amount of the anti-TSLP-R antibody, or antigen-binding fragment thereof, at least once every 3 months. In some embodiments, the method of treating CRSwNP comprises administering to the subject a therapeutically effective amount of the anti-TSLP-R antibody, or antigen-binding fragment thereof, at least once every 4 months. In some embodiments, the method of treating CRSwNP comprises administering to the subject a therapeutically effective amount of the anti-TSLP-R antibody, or antigen-binding fragment thereof, at least once every 5 months. In some embodiments, the method of treating CRSwNP comprises administering to the subject a therapeutically effective amount of the anti-TSLP-R antibody, or antigen-binding fragment thereof, at least once every 6 months. In some embodiments, the method of treating CRSwNP comprises administering to the subject a therapeutically effective amount of the anti-TSLP-R antibody, or antigen-binding fragment thereof, at least once every 7 months. In some embodiments, the method of treating CRSwNP comprises administering to the subject a therapeutically effective amount of the anti-TSLP-R antibody, or antigen-binding fragment thereof, at least once every 8 months. In some embodiments, the method of treating CRSwNP comprises administering to the subject a therapeutically effective amount of the anti-TSLP-R antibody, or antigen-binding fragment thereof, at least once every 9 months. In some embodiments, the method of treating CRSwNP comprises administering to the subject a therapeutically effective amount of the anti-TSLP-R antibody, or antigen-binding fragment thereof, at least once every 10 months. In some embodiments, the method of treating CRSwNP comprises administering to the subject a therapeutically effective amount of the anti-TSLP-R antibody, or antigen-binding fragment thereof, at least once every 11 months. In some embodiments, the method of treating CRSwNP comprises administering to the subject a therapeutically effective amount of the anti-TSLP-R antibody, or antigen-binding fragment thereof, at least once every 12 months.

In some embodiments, the method of treating CRSwNP comprises administering to the subject a therapeutically effective amount of the anti-TSLP-R antibody, or antigen-binding fragment thereof, at an initial dose of about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, or 700 mg; and wherein the anti-TSLP-R antibody, or antigen-binding fragment thereof, is administered every 1 week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, every 20 weeks, every 21 weeks, every 22 weeks, every 23 weeks, every 24 weeks, every 25 weeks, every 26 weeks, every 27 weeks, every 28 weeks, every 29 weeks, every 30 weeks, every 31 weeks, every 32 weeks, every 33 weeks, every 34 weeks, every 35 weeks, every 36 weeks, every 37 weeks, every 38 weeks, every 39 weeks, every 40 weeks, every 41 weeks, every 42 weeks, every 43 weeks, every 44 weeks, every 45 weeks, every 46 weeks, every 47 weeks, every 48 weeks, every 49 weeks, every 50 weeks, every 51 weeks, or every 52 weeks.

In some embodiments, a pharmaceutical dosage of about 20 mg, 25 mg, 50 mg, 100 mg, 150 mg, 200 mg, 250 mg, 300 mg, or 400 mg of the anti-TSLP-R antibody, or antigen-binding fragment thereof, is administered to the subject at a frequency of at least once every 1 week. In some embodiments, a pharmaceutical dosage of about 20 mg of the anti-TSLP-R antibody, or antigen-binding fragment thereof, is administered to the subject at a frequency of at least once every 1 week. In some embodiments, a pharmaceutical dosage of about 25 mg of the anti-TSLP-R antibody, or antigen-binding fragment thereof, is administered to the subject at a frequency of at least once every 1 week. In some embodiments, a pharmaceutical dosage of about 50 mg of the anti-TSLP-R antibody, or antigen-binding fragment thereof, is administered to the subject at a frequency of at least once every 1 week. In some embodiments, a pharmaceutical dosage of about 100 mg of the anti-TSLP-R antibody, or antigen-binding fragment thereof, is administered to the subject at a frequency of at least once every 1 week. In some embodiments, a pharmaceutical dosage of about 150 mg of the anti-TSLP-R antibody, or antigen-binding fragment thereof, is administered to the subject at a frequency of at least once every 1 week. In some embodiments, a pharmaceutical dosage of about 200 mg of the anti-TSLP-R antibody, or antigen-binding fragment thereof, is administered to the subject at a frequency of at least once every 1 week. In some embodiments, a pharmaceutical dosage of about 250 mg of the anti-TSLP-R antibody, or antigen-binding fragment thereof, is administered to the subject at a frequency of at least once every 1 week. In some embodiments, a pharmaceutical dosage of about 300 mg of the anti-TSLP-R antibody, or antigen-binding fragment thereof, is administered to the subject at a frequency of at least once every 1 week. In some embodiments, a pharmaceutical dosage of about 400 mg of the anti-TSLP-R antibody, or antigen-binding fragment thereof, is administered to the subject at a frequency of at least once every 1 week.

In some embodiments, a pharmaceutical dosage of about 20 mg, 25 mg, 50 mg, 100 mg, 150 mg, 200 mg, 250 mg, 300 mg, or 400 mg of the anti-TSLP-R antibody, or antigen-binding fragment thereof, is administered to the subject at a frequency of at least once every 2 weeks. In some embodiments, a pharmaceutical dosage of about 20 mg of the anti-TSLP-R antibody, or antigen-binding fragment thereof, is administered to the subject at a frequency of at least once every 2 weeks. In some embodiments, a pharmaceutical dosage of about 25 mg of the anti-TSLP-R antibody, or antigen-binding fragment thereof, is administered to the subject at a frequency of at least once every 2 weeks. In some embodiments, a pharmaceutical dosage of about 50 mg of the anti-TSLP-R antibody, or antigen-binding fragment thereof, is administered to the subject at a frequency of at least once every 2 weeks. In some embodiments, a pharmaceutical dosage of about 100 mg of the anti-TSLP-R antibody, or antigen-binding fragment thereof, is administered to the subject at a frequency of at least once every 2 weeks. In some embodiments, a pharmaceutical dosage of about 150 mg of the anti-TSLP-R antibody, or antigen-binding fragment thereof, is administered to the subject at a frequency of at least once every 2 weeks. In some embodiments, a pharmaceutical dosage of about 200 mg of the anti-TSLP-R antibody, or antigen-binding fragment thereof, is administered to the subject at a frequency of at least once every 2 weeks. In some embodiments, a pharmaceutical dosage of about 250 mg of the anti-TSLP-R antibody, or antigen-binding fragment thereof, is administered to the subject at a frequency of at least once every 2 weeks. In some embodiments, a pharmaceutical dosage of about 300 mg of the anti-TSLP-R antibody, or antigen-binding fragment thereof, is administered to the subject at a frequency of at least once every 2 weeks. In some embodiments, a pharmaceutical dosage of about 400 mg of the anti-TSLP-R antibody, or antigen-binding fragment thereof, is administered to the subject at a frequency of at least once every 2 weeks.

In some embodiments, a pharmaceutical dosage of about 20 mg, 25 mg, 50 mg, 100 mg, 150 mg, 200 mg, 250 mg, 300 mg, or 400 mg of the anti-TSLP-R antibody, or antigen-binding fragment thereof, is administered to the subject at a frequency of at least once every 4 weeks. In some embodiments, a pharmaceutical dosage of about 20 mg of the anti-TSLP-R antibody, or antigen-binding fragment thereof, is administered to the subject at a frequency of at least once every 4 weeks. In some embodiments, a pharmaceutical dosage of about 25 mg of the anti-TSLP-R antibody, or antigen-binding fragment thereof, is administered to the subject at a frequency of at least once every 4 weeks. In some embodiments, a pharmaceutical dosage of about 50 mg of the anti-TSLP-R antibody, or antigen-binding fragment thereof, is administered to the subject at a frequency of at least once every 4 weeks. In some embodiments, a pharmaceutical dosage of about 100 mg of the anti-TSLP-R antibody, or antigen-binding fragment thereof, is administered to the subject at a frequency of at least once every 4 weeks. In some embodiments, a pharmaceutical dosage of about 150 mg of the anti-TSLP-R antibody, or antigen-binding fragment thereof, is administered to the subject at a frequency of at least once every 4 weeks. In some embodiments, a pharmaceutical dosage of about 200 mg of the anti-TSLP-R antibody, or antigen-binding fragment thereof, is administered to the subject at a frequency of at least once every 4 weeks. In some embodiments, a pharmaceutical dosage of about 250 mg of the anti-TSLP-R antibody, or antigen-binding fragment thereof, is administered to the subject at a frequency of at least once every 4 weeks. In some embodiments, a pharmaceutical dosage of about 300 mg of the anti-TSLP-R antibody, or antigen-binding fragment thereof, is administered to the subject at a frequency of at least once every 4 weeks. In some embodiments, a pharmaceutical dosage of about 400 mg of the anti-TSLP-R antibody, or antigen-binding fragment thereof, is administered to the subject at a frequency of at least once every 4 weeks.

In some embodiments, a pharmaceutical dosage of about 20 mg, 25 mg, 50 mg, 100 mg, 150 mg, 200 mg, 250 mg, 300 mg, or 400 mg of the anti-TSLP-R antibody, or antigen-binding fragment thereof, is administered to the subject at a frequency of at least once every 8 weeks. In some embodiments, a pharmaceutical dosage of about 20 mg of the anti-TSLP-R antibody, or antigen-binding fragment thereof, is administered to the subject at a frequency of at least once every 8 weeks. In some embodiments, a pharmaceutical dosage of about 25 mg of the anti-TSLP-R antibody, or antigen-binding fragment thereof, is administered to the subject at a frequency of at least once every 8 weeks. In some embodiments, a pharmaceutical dosage of about 50 mg of the anti-TSLP-R antibody, or antigen-binding fragment thereof, is administered to the subject at a frequency of at least once every 8 weeks. In some embodiments, a pharmaceutical dosage of about 100 mg of the anti-TSLP-R antibody, or antigen-binding fragment thereof, is administered to the subject at a frequency of at least once every 8 weeks. In some embodiments, a pharmaceutical dosage of about 150 mg of the anti-TSLP-R antibody, or antigen-binding fragment thereof, is administered to the subject at a frequency of at least once every 8 weeks. In some embodiments, a pharmaceutical dosage of about 200 mg of the anti-TSLP-R antibody, or antigen-binding fragment thereof, is administered to the subject at a frequency of at least once every 8 weeks. In some embodiments, a pharmaceutical dosage of about 250 mg of the anti-TSLP-R antibody, or antigen-binding fragment thereof, is administered to the subject at a frequency of at least once every 8 weeks. In some embodiments, a pharmaceutical dosage of about 300 mg of the anti-TSLP-R antibody, or antigen-binding fragment thereof, is administered to the subject at a frequency of at least once every 8 weeks. In some embodiments, a pharmaceutical dosage of about 400 mg of the anti-TSLP-R antibody, or antigen-binding fragment thereof, is administered to the subject at a frequency of at least once every 8 weeks.

In some embodiments, a pharmaceutical dosage of about 20 mg, 25 mg, 50 mg, 100 mg, 150 mg, 200 mg, 250 mg, 300 mg, or 400 mg of the anti-TSLP-R antibody, or antigen-binding fragment thereof, is administered to the subject at a frequency of at least once every 12 weeks. In some embodiments, a pharmaceutical dosage of about 20 mg of the anti-TSLP-R antibody, or antigen-binding fragment thereof, is administered to the subject at a frequency of at least once every 12 weeks. In some embodiments, a pharmaceutical dosage of about 25 mg of the anti-TSLP-R antibody, or antigen-binding fragment thereof, is administered to the subject at a frequency of at least once every 12 weeks. In some embodiments, a pharmaceutical dosage of about 50 mg of the anti-TSLP-R antibody, or antigen-binding fragment thereof, is administered to the subject at a frequency of at least once every 12 weeks. In some embodiments, a pharmaceutical dosage of about 100 mg of the anti-TSLP-R antibody, or antigen-binding fragment thereof, is administered to the subject at a frequency of at least once every 12 weeks. In some embodiments, a pharmaceutical dosage of about 150 mg of the anti-TSLP-R antibody, or antigen-binding fragment thereof, is administered to the subject at a frequency of at least once every 12 weeks. In some embodiments, a pharmaceutical dosage of about 200 mg of the anti-TSLP-R antibody, or antigen-binding fragment thereof, is administered to the subject at a frequency of at least once every 12 weeks. In some embodiments, a pharmaceutical dosage of about 250 mg of the anti-TSLP-R antibody, or antigen-binding fragment thereof, is administered to the subject at a frequency of at least once every 12 weeks. In some embodiments, a pharmaceutical dosage of about 300 mg of the anti-TSLP-R antibody, or antigen-binding fragment thereof, is administered to the subject at a frequency of at least once every 12 weeks. In some embodiments, a pharmaceutical dosage of about 400 mg of the anti-TSLP-R antibody, or antigen-binding fragment thereof, is administered to the subject at a frequency of at least once every 12 weeks.

In some embodiments, a pharmaceutical dosage of about 100 mg, 200 mg, 250 mg, 300 mg, 350 mg, 400 mg, 500 mg, 600 mg, 700 mg, or 800 mg of the anti-TSLP-R antibody, or antigen-binding fragment thereof, is administered to the subject at a frequency of at least once every 16 weeks. In some embodiments, a pharmaceutical dosage of about 100 mg of the anti-TSLP-R antibody, or antigen-binding fragment thereof, is administered to the subject at a frequency of at least once every 16 weeks. In some embodiments, a pharmaceutical dosage of about 200 mg of the anti-TSLP-R antibody, or antigen-binding fragment thereof, is administered to the subject at a frequency of at least once every 16 weeks. In some embodiments, a pharmaceutical dosage of about 250 mg of the anti-TSLP-R antibody, or antigen-binding fragment thereof, is administered to the subject at a frequency of at least once every 16 weeks. In some embodiments, a pharmaceutical dosage of about 300 mg of the anti-TSLP-R antibody, or antigen-binding fragment thereof, is administered to the subject at a frequency of at least once every 16 weeks. In some embodiments, a pharmaceutical dosage of about 350 mg of the anti-TSLP-R antibody, or antigen-binding fragment thereof, is administered to the subject at a frequency of at least once every 16 weeks. In some embodiments, a pharmaceutical dosage of about 400 mg of the anti-TSLP-R antibody, or antigen-binding fragment thereof, is administered to the subject at a frequency of at least once every 16 weeks. In some embodiments, a pharmaceutical dosage of about 500 mg of the anti-TSLP-R antibody, or antigen-binding fragment thereof, is administered to the subject at a frequency of at least once every 16 weeks. In some embodiments, a pharmaceutical dosage of about 600 mg of the anti-TSLP-R antibody, or antigen-binding fragment thereof, is administered to the subject at a frequency of at least once every 16 weeks. In some embodiments, a pharmaceutical dosage of about 700 mg of the anti-TSLP-R antibody, or antigen-binding fragment thereof, is administered to the subject at a frequency of at least once every 16 weeks. In some embodiments, a pharmaceutical dosage of about 800 mg of the anti-TSLP-R antibody, or antigen-binding fragment thereof, is administered to the subject at a frequency of at least once every 16 weeks.

In some embodiments, a pharmaceutical dosage of about 200 mg, 250 mg, 300 mg, 350 mg, 400 mg, 500 mg, 600 mg, 700 mg, or 800 mg of the anti-TSLP-R antibody, or antigen-binding fragment thereof, is administered to the subject at a frequency of at least once every 24 weeks. In some embodiments, a pharmaceutical dosage of about 100 mg of the anti-TSLP-R antibody, or antigen-binding fragment thereof, is administered to the subject at a frequency of at least once every 24 weeks. In some embodiments, a pharmaceutical dosage of about 200 mg of the anti-TSLP-R antibody, or antigen-binding fragment thereof, is administered to the subject at a frequency of at least once every 24 weeks. In some embodiments, a pharmaceutical dosage of about 250 mg of the anti-TSLP-R antibody, or antigen-binding fragment thereof, is administered to the subject at a frequency of at least once every 24 weeks. In some embodiments, a pharmaceutical dosage of about 300 mg of the anti-TSLP-R antibody, or antigen-binding fragment thereof, is administered to the subject at a frequency of at least once every 24 weeks. In some embodiments, a pharmaceutical dosage of about 400 mg of the anti-TSLP-R antibody, or antigen-binding fragment thereof, is administered to the subject at a frequency of at least once every 24 weeks. In some embodiments, a pharmaceutical dosage of about 500 mg of the anti-TSLP-R antibody, or antigen-binding fragment thereof, is administered to the subject at a frequency of at least once every 24 weeks. In some embodiments, a pharmaceutical dosage of about 600 mg of the anti-TSLP-R antibody, or antigen-binding fragment thereof, is administered to the subject at a frequency of at least once every 24 weeks. In some embodiments, a pharmaceutical dosage of about 700 mg of the anti-TSLP-R antibody, or antigen-binding fragment thereof, is administered to the subject at a frequency of at least once every 25 weeks. In some embodiments, a pharmaceutical dosage of about 800 mg of the anti-TSLP-R antibody, or antigen-binding fragment thereof, is administered to the subject at a frequency of at least once every 24 weeks.

In some embodiments, the anti-TSLP-R antibody, or antigen-binding fragment thereof, is administered for a total of 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks.

In some embodiments, the anti-TSLP-R antibody, or antigen-binding fragment thereof, is administered every 1 week for a total of at least 1 week from the first administration of the anti-TSLP-R antibody. In some embodiments, the anti-TSLP-R antibody, or antigen-binding fragment thereof, is administered every 1 week for a total of at least 2 weeks from the first administration of the anti-TSLP-R antibody. In some embodiments, the anti-TSLP-R antibody, or antigen-binding fragment thereof, is administered every 2 weeks for a total of at least 2 weeks from the first administration of the anti-TSLP-R antibody. In some embodiments, the anti-TSLP-R antibody, or antigen-binding fragment thereof, is administered every 1 week for a total of at least 3 weeks from the first administration of the anti-TSLP-R antibody.

In some embodiments, the anti-TSLP-R antibody, or antigen-binding fragment thereof, is administered every 1 week for a total of at least 4 weeks from the first administration of the anti-TSLP-R antibody. In some embodiments, the anti-TSLP-R antibody, or antigen-binding fragment thereof, is administered every 2 weeks for a total of at least 4 weeks from the first administration of the anti-TSLP-R antibody. In some embodiments, the anti-TSLP-R antibody, or antigen-binding fragment thereof, is administered every 4 weeks for a total of at least 4 weeks from the first administration of the anti-TSLP-R antibody.

In some embodiments, the anti-TSLP-R antibody, or antigen-binding fragment thereof, is administered every 1 week for a total of at least 5 weeks from the first administration of the anti-TSLP-R antibody. In some embodiments, the anti-TSLP-R antibody, or antigen-binding fragment thereof, is administered every 1 week for a total of at least 6 weeks from the first administration of the anti-TSLP-R antibody. In some embodiments, the anti-TSLP-R antibody, or antigen-binding fragment thereof, is administered every 2 weeks for a total of at least 6 weeks from the first administration of the anti-TSLP-R antibody. In some embodiments, the anti-TSLP-R antibody, or antigen-binding fragment thereof, is administered every 1 week for a total of at least 7 weeks from the first administration of the anti-TSLP-R antibody.

In some embodiments, the anti-TSLP-R antibody, or antigen-binding fragment thereof, is administered every 1 week for a total of at least 8 weeks from the first administration of the anti-TSLP-R antibody. In some embodiments, the anti-TSLP-R antibody, or antigen-binding fragment thereof, is administered every 2 weeks for a total of at least 8 weeks from the first administration of the anti-TSLP-R antibody. In some embodiments, the anti-TSLP-R antibody, or antigen-binding fragment thereof, is administered every 4 weeks for a total of at least 8 weeks from the first administration of the anti-TSLP-R antibody. In some embodiments, the anti-TSLP-R antibody, or antigen-binding fragment thereof, is administered every 8 weeks for a total of at least 8 weeks from the first administration of the anti-TSLP-R antibody.

In some embodiments, the anti-TSLP-R antibody, or antigen-binding fragment thereof, is administered every 1 week for a total of at least 9 weeks from the first administration of the anti-TSLP-R antibody. In some embodiments, the anti-TSLP-R antibody, or antigen-binding fragment thereof, is administered every 1 week for a total of at least 10 weeks from the first administration of the anti-TSLP-R antibody. In some embodiments, the anti-TSLP-R antibody, or antigen-binding fragment thereof, is administered every 2 weeks for a total of at least 10 weeks from the first administration of the anti-TSLP-R antibody. In some embodiments, the anti-TSLP-R antibody, or antigen-binding fragment thereof, is administered every 1 week for a total of at least 11 weeks from the first administration of the anti-TSLP-R antibody.

In some embodiments, the anti-TSLP-R antibody, or antigen-binding fragment thereof, is administered every 1 week for a total of at least 12 weeks from the first administration of the anti-TSLP-R antibody. In some embodiments, the anti-TSLP-R antibody, or antigen-binding fragment thereof, is administered every 2 weeks for a total of at least 12 weeks from the first administration of the anti-TSLP-R antibody. In some embodiments, the anti-TSLP-R antibody, or antigen-binding fragment thereof, is administered every 4 weeks for a total of at least 12 weeks from the first administration of the anti-TSLP-R antibody. In some embodiments, the anti-TSLP-R antibody, or antigen-binding fragment thereof, is administered every 12 weeks for a total of at least 12 weeks from the first administration of the anti-TSLP-R antibody.

In some embodiments, the anti-TSLP-R antibody, or antigen-binding fragment thereof, is administered every 1 week for a total of at least 13 weeks from the first administration of the anti-TSLP-R antibody. In some embodiments, the anti-TSLP-R antibody, or antigen-binding fragment thereof, is administered every 1 week for a total of at least 14 weeks from the first administration of the anti-TSLP-R antibody. In some embodiments, the anti-TSLP-R antibody, or antigen-binding fragment thereof, is administered every 2 weeks for a total of at least 14 weeks from the first administration of the anti-TSLP-R antibody. In some embodiments, the anti-TSLP-R antibody, or antigen-binding fragment thereof, is administered every 1 week for a total of at least 15 weeks from the first administration of the anti-TSLP-R antibody.

In some embodiments, the anti-TSLP-R antibody, or antigen-binding fragment thereof, is administered every 1 week for a total of at least 16 weeks from the first administration of the anti-TSLP-R antibody. In some embodiments, the anti-TSLP-R antibody, or antigen-binding fragment thereof, is administered every 2 weeks for a total of at least 16 weeks from the first administration of the anti-TSLP-R antibody. In some embodiments, the anti-TSLP-R antibody, or antigen-binding fragment thereof, is administered every 4 weeks for a total of at least 16 weeks from the first administration of the anti-TSLP-R antibody. In some embodiments, the anti-TSLP-R antibody, or antigen-binding fragment thereof, is administered every 8 weeks for a total of at least 16 weeks from the first administration of the anti-TSLP-R antibody. In some embodiments, the anti-TSLP-R antibody, or antigen-binding fragment thereof, is administered every 16 weeks for a total of at least 16 weeks from the first administration of the anti-TSLP-R antibody.

In some embodiments, the anti-TSLP-R antibody, or antigen-binding fragment thereof, is administered every 1 week for a total of at least 17 weeks from the first administration of the anti-TSLP-R antibody. In some embodiments, the anti-TSLP-R antibody, or antigen-binding fragment thereof, is administered every 1 week for a total of at least 18 weeks from the first administration of the anti-TSLP-R antibody. In some embodiments, the anti-TSLP-R antibody, or antigen-binding fragment thereof, is administered every 2 weeks for a total of at least 18 weeks from the first administration of the anti-TSLP-R antibody. In some embodiments, the anti-TSLP-R antibody, or antigen-binding fragment thereof, is administered every 1 week for a total of at least 19 weeks from the first administration of the anti-TSLP-R antibody.

In some embodiments, the anti-TSLP-R antibody, or antigen-binding fragment thereof, is administered every 1 week for a total of at least 20 weeks from the first administration of the anti-TSLP-R antibody. In some embodiments, the anti-TSLP-R antibody, or antigen-binding fragment thereof, is administered every 2 weeks for a total of at least 20 weeks from the first administration of the anti-TSLP-R antibody. In some embodiments, the anti-TSLP-R antibody, or antigen-binding fragment thereof, is administered every 4 weeks for at least 20 weeks.

In some embodiments, the anti-TSLP-R antibody, or antigen-binding fragment thereof, is administered every 1 week for a total of at least 21 weeks from the first administration of the anti-TSLP-R antibody. In some embodiments, the anti-TSLP-R antibody, or antigen-binding fragment thereof, is administered every 1 week for a total of at least 22 weeks from the first administration of the anti-TSLP-R antibody. In some embodiments, the anti-TSLP-R antibody, or antigen-binding fragment thereof, is administered every 2 weeks for a total of at least 22 weeks from the first administration of the anti-TSLP-R antibody. In some embodiments, the anti-TSLP-R antibody, or antigen-binding fragment thereof, is administered every 1 week for a total of at least 23 weeks from the first administration of the anti-TSLP-R antibody.

In some embodiments, the anti-TSLP-R antibody, or antigen-binding fragment thereof, is administered every 1 week for a total of at least 24 weeks from the first administration of the anti-TSLP-R antibody. In some embodiments, the anti-TSLP-R antibody, or antigen-binding fragment thereof, is administered every 2 weeks for a total of at least 24 weeks from the first administration of the anti-TSLP-R antibody. In some embodiments, the anti-TSLP-R antibody, or antigen-binding fragment thereof, is administered every 4 weeks for at least 24 weeks. In some embodiments, the anti-TSLP-R antibody, or antigen-binding fragment thereof, is administered every 8 weeks for at least 24 weeks. In some embodiments, the anti-TSLP-R antibody, or antigen-binding fragment thereof, is administered every 12 weeks for at least 24 weeks. In some embodiments, the anti-TSLP-R antibody, or antigen-binding fragment thereof, is administered every 24 weeks for at least 24 weeks.

In some embodiments, the anti-TSLP-R antibody, or antigen-binding fragment thereof, is administered every 1 week for a total of at least 25 weeks from the first administration of the anti-TSLP-R antibody. In some embodiments, the anti-TSLP-R antibody, or antigen-binding fragment thereof, is administered every 1 week for a total of at least 26 weeks from the first administration of the anti-TSLP-R antibody. In some embodiments, the anti-TSLP-R antibody, or antigen-binding fragment thereof, is administered every 2 weeks for a total of at least 26 weeks from the first administration of the anti-TSLP-R antibody. In some embodiments, the anti-TSLP-R antibody, or antigen-binding fragment thereof, is administered every 1 week for a total of at least 27 weeks from the first administration of the anti-TSLP-R antibody.

In some embodiments, the anti-TSLP-R antibody, or antigen-binding fragment thereof, is administered every 1 week for a total of at least 28 weeks from the first administration of the anti-TSLP-R antibody. In some embodiments, the anti-TSLP-R antibody, or antigen-binding fragment thereof, is administered every 2 weeks for a total of at least 28 weeks from the first administration of the anti-TSLP-R antibody. In some embodiments, the anti-TSLP-R antibody, or antigen-binding fragment thereof, is administered every 4 weeks for a total of at least 28 weeks from the first administration of the anti-TSLP-R antibody.

In some embodiments, the anti-TSLP-R antibody, or antigen-binding fragment thereof, is administered every 1 week for a total of at least 29 weeks from the first administration of the anti-TSLP-R antibody. In some embodiments, the anti-TSLP-R antibody, or antigen-binding fragment thereof, is administered every 1 week for a total of at least 30 weeks from the first administration of the anti-TSLP-R antibody. In some embodiments, the anti-TSLP-R antibody, or antigen-binding fragment thereof, is administered every 2 weeks for a total of at least 30 weeks from the first administration of the anti-TSLP-R antibody. In some embodiments, the anti-TSLP-R antibody, or antigen-binding fragment thereof, is administered every 1 week for a total of at least 31 weeks from the first administration of the anti-TSLP-R antibody.

In some embodiments, the anti-TSLP-R antibody, or antigen-binding fragment thereof, is administered every 1 week for a total of at least 32 weeks from the first administration of the anti-TSLP-R antibody. In some embodiments, the anti-TSLP-R antibody, or antigen-binding fragment thereof, is administered every 2 weeks for a total of at least 32 weeks from the first administration of the anti-TSLP-R antibody. In some embodiments, the anti-TSLP-R antibody, or antigen-binding fragment thereof, is administered every 4 weeks for a total of at least 32 weeks from the first administration of the anti-TSLP-R antibody. In some embodiments, the anti-TSLP-R antibody, or antigen-binding fragment thereof, is administered every 8 weeks for a total of at least 32 weeks from the first administration of the anti-TSLP-R antibody. In some embodiments, the anti-TSLP-R antibody, or antigen-binding fragment thereof, is administered every 16 weeks for a total of at least 32 weeks from the first administration of the anti-TSLP-R antibody.

In some embodiments, the anti-TSLP-R antibody, or antigen-binding fragment thereof, is administered every 1 week for a total of at least 33 weeks from the first administration of the anti-TSLP-R antibody. In some embodiments, the anti-TSLP-R antibody, or antigen-binding fragment thereof, is administered every 1 week for a total of at least 34 weeks from the first administration of the anti-TSLP-R antibody. In some embodiments, the anti-TSLP-R antibody, or antigen-binding fragment thereof, is administered every 2 weeks for a total of at least 34 weeks from the first administration of the anti-TSLP-R antibody. In some embodiments, the anti-TSLP-R antibody, or antigen-binding fragment thereof, is administered every 1 week for a total of at least 35 weeks from the first administration of the anti-TSLP-R antibody.

In some embodiments, the anti-TSLP-R antibody, or antigen-binding fragment thereof, is administered every 1 week for a total of at least 36 weeks from the first administration of the anti-TSLP-R antibody. In some embodiments, the anti-TSLP-R antibody, or antigen-binding fragment thereof, is administered every 2 weeks for a total of at least 36 weeks from the first administration of the anti-TSLP-R antibody. In some embodiments, the anti-TSLP-R antibody, or antigen-binding fragment thereof, is administered every 4 for a total of at least 36 weeks from the first administration of the anti-TSLP-R antibody. In some embodiments, the anti-TSLP-R antibody, or antigen-binding fragment thereof, is administered every 12 weeks for a total of at least 36 weeks from the first administration of the anti-TSLP-R antibody.

In some embodiments, the anti-TSLP-R antibody, or antigen-binding fragment thereof, is administered every 1 week for a total of at least 37 weeks from the first administration of the anti-TSLP-R antibody. In some embodiments, the anti-TSLP-R antibody, or antigen-binding fragment thereof, is administered every 1 week for a total of at least 38 weeks from the first administration of the anti-TSLP-R antibody. In some embodiments, the anti-TSLP-R antibody, or antigen-binding fragment thereof, is administered every 2 weeks for a total of at least 38 weeks from the first administration of the anti-TSLP-R antibody. In some embodiments, the anti-TSLP-R antibody, or antigen-binding fragment thereof, is administered every 1 week for a total of at least 39 weeks from the first administration of the anti-TSLP-R antibody.

In some embodiments, the anti-TSLP-R antibody, or antigen-binding fragment thereof, is administered every 1 week for a total of at least 40 weeks from the first administration of the anti-TSLP-R antibody. In some embodiments, the anti-TSLP-R antibody, or antigen-binding fragment thereof, is administered every 2 weeks for a total of at least 40 weeks from the first administration of the anti-TSLP-R antibody. In some embodiments, the anti-TSLP-R antibody, or antigen-binding fragment thereof, is administered every 4 weeks for a total of at least 40 weeks from the first administration of the anti-TSLP-R antibody. In some embodiments, the anti-TSLP-R antibody, or antigen-binding fragment thereof, is administered every 8 weeks for a total of at least 40 weeks from the first administration of the anti-TSLP-R antibody.

In some embodiments, the anti-TSLP-R antibody, or antigen-binding fragment thereof, is administered every 1 week for a total of at least 41 weeks from the first administration of the anti-TSLP-R antibody. In some embodiments, the anti-TSLP-R antibody, or antigen-binding fragment thereof, is administered every 1 week for a total of at least 42 weeks from the first administration of the anti-TSLP-R antibody. In some embodiments, the anti-TSLP-R antibody, or antigen-binding fragment thereof, is administered every 2 weeks for a total of at least 42 weeks from the first administration of the anti-TSLP-R antibody. In some embodiments, the anti-TSLP-R antibody, or antigen-binding fragment thereof, is administered every 1 week for a total of at least 43 weeks from the first administration of the anti-TSLP-R antibody.

In some embodiments, the anti-TSLP-R antibody, or antigen-binding fragment thereof, is administered every 1 week for a total of at least 44 weeks from the first administration of the anti-TSLP-R antibody. In some embodiments, the anti-TSLP-R antibody, or antigen-binding fragment thereof, is administered every 2 weeks for a total of at least 44 weeks from the first administration of the anti-TSLP-R antibody. In some embodiments, the anti-TSLP-R antibody, or antigen-binding fragment thereof, is administered every 4 weeks for a total of at least 44 weeks from the first administration of the anti-TSLP-R antibody.

In some embodiments, the anti-TSLP-R antibody, or antigen-binding fragment thereof, is administered every 1 week for a total of at least 45 weeks from the first administration of the anti-TSLP-R antibody. In some embodiments, the anti-TSLP-R antibody, or antigen-binding fragment thereof, is administered every 1 week for a total of at least 46 weeks from the first administration of the anti-TSLP-R antibody. In some embodiments, the anti-TSLP-R antibody, or antigen-binding fragment thereof, is administered every 2 weeks for a total of at least 46 weeks from the first administration of the anti-TSLP-R antibody. In some embodiments, the anti-TSLP-R antibody, or antigen-binding fragment thereof, is administered every 1 week for a total of at least 47 weeks from the first administration of the anti-TSLP-R antibody.

In some embodiments, the anti-TSLP-R antibody, or antigen-binding fragment thereof, is administered every 1 week for a total of at least 48 weeks from the first administration of the anti-TSLP-R antibody. In some embodiments, the anti-TSLP-R antibody, or antigen-binding fragment thereof, is administered every 2 weeks for a total of at least 48 weeks from the first administration of the anti-TSLP-R antibody. In some embodiments, the anti-TSLP-R antibody, or antigen-binding fragment thereof, is administered every 4 weeks for a total of at least 48 weeks from the first administration of the anti-TSLP-R antibody. In some embodiments, the anti-TSLP-R antibody, or antigen-binding fragment thereof, is administered every 8 weeks for a total of at least 48 weeks from the first administration of the anti-TSLP-R antibody. In some embodiments, the anti-TSLP-R antibody, or antigen-binding fragment thereof, is administered every 12 weeks for a total of at least 48 weeks from the first administration of the anti-TSLP-R antibody. In some embodiments, the anti-TSLP-R antibody, or antigen-binding fragment thereof, is administered every 24 weeks for a total of at least 48 weeks from the first administration of the anti-TSLP-R antibody.

In some embodiments, the anti-TSLP-R antibody, or antigen-binding fragment thereof, is administered every 1 week for a total of at least 49 weeks from the first administration of the anti-TSLP-R antibody. In some embodiments, the anti-TSLP-R antibody, or antigen-binding fragment thereof, is administered every 1 week for a total of at least 50 weeks from the first administration of the anti-TSLP-R antibody. In some embodiments, the anti-TSLP-R antibody, or antigen-binding fragment thereof, is administered every 2 weeks for a total of at least 50 weeks from the first administration of the anti-TSLP-R antibody. In some embodiments, the anti-TSLP-R antibody, or antigen-binding fragment thereof, is administered every 1 week for a total of at least 51 weeks from the first administration of the anti-TSLP-R antibody.

In some embodiments, the anti-TSLP-R antibody, or antigen-binding fragment thereof, is administered every 1 week for a total of at least 52 weeks from the first administration of the anti-TSLP-R antibody. In some embodiments, the anti-TSLP-R antibody, or antigen-binding fragment thereof, is administered every 2 weeks for a total of at least 52 weeks from the first administration of the anti-TSLP-R antibody. In some embodiments, the anti-TSLP-R antibody, or antigen-binding fragment thereof, is administered every 4 weeks for a total of at least 52 weeks from the first administration of the anti-TSLP-R antibody.

In some embodiments, the method of treating CRSwNP comprises administering to the subject a therapeutically effective amount of the anti-TSLP-R antibody, or antigen-binding fragment thereof, co-administered with a second therapeutic agent. In some embodiments, the method of treating CRSwNP comprises administering the second therapeutic agent by injection.

In some embodiments, the method of treating CRSwNP comprises administering the second therapeutic agent at the same time as, prior to, or following administration of the anti-TSLP-R antibody, or antigen-binding fragment thereof.

In some embodiments, the second therapeutic agent is indicated to treat Chronic Rhinosinusitis with Nasal Polyps. In some embodiments, the anti-TSLP-R antibody, or antigen-binding fragment thereof, is co-administered with a second therapeutic, wherein the second therapeutic agent is selected from: levofloxacin, ciprofloxacin, moxifloxacin, sulfamethoxazole/trimethoprim, acetaminophen/caffeine, amoxicillin, ampicillin, oxacillin, amoxicillin/clavulante, ampicillin/sulbactam, chlorpheniramine/pseudoephedrine, dexchlorpheniramine/dextromethorphan/pseudoephedrine, diphenhydramine/phenylephrine, dextromethorphan/diphenhydramine/phenylephrine, dextromethorphan/phenylephrine/pyrilamine, cefuroxime, cefprozil, cefaclor, cefuroxime, cefprozil, cefaclor, cefdinir, cefixime, cefpodoxime, ceftazidime, clindamycin, prednisone, fluticasone, mometasone, beclomethasone, azithromycin, clarithromycin, omalizumab, dupilumab, mepolizumab, or any combination thereof.

In some embodiments, the second therapeutic agent is a quinolone, a sulfonamide, an analgesic combination, an aminopenicillin, a penicillinase resistant penicillin, a beta-lactamas inhibitor, an upper respiratory combination, a second generation cephalosporin, a third generation cephalosporin, a linomycin derivative, a glucocorticoid, a nasal steroid, a macrolide, or a selective immunosuppressant.

In some embodiments, the second therapeutic agent is an oral corticosteroid or an intranasal corticosteroid. In some embodiments, the second therapeutic agent is an intranasal corticosteroid that comprises mometasone furoate. In some embodiments, the second therapeutic agent is a local treatment. In some embodiments, the second therapeutic agent is a nasal spray.

In some embodiments, the second therapeutic agent is a systemic treatment. In some embodiments, the second therapeutic agent comprising a systemic treatments comprises a biologic. In some embodiments, the biologic can be an antibody. In some embodiments, the antibody can be an interleukin inhibitor, a TSLP inhibitor, or an IgG inhibitor. In some embodiments, the antibody, or antibody fragment thereof, targets IgE. In some embodiments, the second therapeutic agent is an antibody, or fragment thereof, that targets IgE, wherein the antibody that targets IgE is omalizumab, ligelizumab, FB-825, or UB-221. In some embodiments, the antibody, or antibody fragment thereof, that specifically targets an agent indicated in the pathogenesis of Chronic Rhinosinusitis with Nasal Polyps, is an antibody that targets TSLP. In some embodiments, the second therapeutic agent is an antibody, or fragment thereof, that targets TSLP, wherein the antibody that targets TSLP is tezepelumab, CSJ-117, or CM-326. In some embodiments, the second therapeutic agent is siglec. In some embodiments, the second therapeutic agent targets an interleukin. In some embodiments, the second therapeutic agent is an antibody, or antibody fragment thereof, that specifically targets IL-5, IL-13, IL-4, or IL-4/IL-13. In some embodiments, the interleukin inhibitor is an anti-IL-5 antibody, or antigen-binding fragment thereof. In some embodiments, the IL-5 inhibitor is benralizumab, mepolizumab, reslizumab, or 610 (610-SBio). In some embodiments, the interleukin inhibitor is an anti-IL-13 antibody, or antigen-binding fragment thereof. In some embodiments, the IL-13 inhibitor is dupilumab. In some embodiments, the interleukin inhibitor is an anti-IL-4 antibody, or antigen-binding fragment thereof. In some embodiments, the IL-4 inhibitor is dupilumab. In some embodiments, the interleukin inhibitor inhibits two or more interleukins. In some embodiments, the interleukin inhibitor is an antibody, or antigen-binding fragment thereof, that targets one or more interleukins. In some embodiments, the interleukin inhibitor is an antibody, or antigen-binding fragment thereof, that targets two interleukins. In some embodiments, the interleukin inhibitor is an anti-IL-4/IL-13 antibody, or antigen-binding fragment thereof. In some embodiments, the IL-4/IL-13 inhibitor is dupilumab, eblasakimab, cendakimab, CBP-201, CM-310, AK-120, BC005, QX-005N, or 611 (611-SBio).

In some embodiments, the method of treating CRSwNP comprises administering the anti-TSLP-R antibody, or antigen-binding fragment thereof, to a subject diagnosed with CRSwNP, wherein the subject has a history of inadequate response to treatment with intranasal corticosteroids In some embodiments, the method of treating CRSwNP comprises administering the anti-TSLP-R antibody, or antigen-binding fragment thereof, to a subject diagnosed with CRSwNP, wherein the subject has one or more nasal polyp present in each nostril prior to the administering of the anti-TSLP-R antibody, or antigen-binding fragment thereof. In some embodiments, the method of treating CRSwNP comprises administering the anti-TSLP-R antibody, or antigen-binding fragment thereof, to a subject diagnosed with CRSwNP, wherein the subject has a total of at least 5 nasal polyps, with two or more nasal polyps present in each nostril prior to the administering of the anti-TSLP-R antibody, or antigen-binding fragment thereof. In some embodiments, the method of treating CRSwNP comprises administering the anti-TSLP-R antibody, or antigen-binding fragment thereof, to a subject diagnosed with CRSwNP, wherein the subject has previously had a relapse of nasal polyposis after receiving surgery to treat the polyps and prior to the administering of the anti-TSLP-R antibody, or antigen-binding fragment thereof.

In some embodiments, the subject has an improvement of one or more symptom of CRSwNP following administration of the anti-TSLP-R antibody, or antigen-binding fragment thereof. In some embodiments, the one or more symptom of CRSwNP is improved after, or about, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of treatment with the anti-TSLP-R antibody, or antigen-binding fragment thereof. In some embodiments, the one or more symptom of CRSwNP is improved after, or about, 1 week of treatment with the anti-TSLP-R antibody, or antigen-binding fragment thereof. In some embodiments, the one or more symptom of CRSwNP is improved after, or about, 2 weeks of treatment with the anti-TSLP-R antibody, or antigen-binding fragment thereof. In some embodiments, the one or more symptom of CRSwNP is improved after, or about, 3 weeks of treatment with the anti-TSLP-R antibody, or antigen-binding fragment thereof. In some embodiments, the one or more symptom of CRSwNP is improved after, or about, 4 weeks of treatment with the anti-TSLP-R antibody, or antigen-binding fragment thereof. In some embodiments, the one or more symptom of CRSwNP is improved after, or about, 5 weeks of treatment with the anti-TSLP-R antibody, or antigen-binding fragment thereof. In some embodiments, the one or more symptom of CRSwNP is improved after, or about, 6 weeks of treatment with the anti-TSLP-R antibody, or antigen-binding fragment thereof. In some embodiments, the one or more symptom of CRSwNP is improved after, or about, 7 weeks of treatment with the anti-TSLP-R antibody, or antigen-binding fragment thereof. In some embodiments, the one or more symptom of CRSwNP is improved after, or about, 8 weeks of treatment with the anti-TSLP-R antibody, or antigen-binding fragment thereof. In some embodiments, the one or more symptom of CRSwNP is improved after, or about, 9 weeks of treatment with the anti-TSLP-R antibody, or antigen-binding fragment thereof. In some embodiments, the one or more symptom of CRSwNP is improved after, or about, 10 weeks of treatment with the anti-TSLP-R antibody, or antigen-binding fragment thereof. In some embodiments, the one or more symptom of CRSwNP is improved after, or about, 11 weeks of treatment with the anti-TSLP-R antibody, or antigen-binding fragment thereof. In some embodiments, the one or more symptom of CRSwNP is improved after, or about, 12 weeks of treatment with the anti-TSLP-R antibody, or antigen-binding fragment thereof. In some embodiments, the one or more symptom of CRSwNP is improved after, or about, 13 weeks of treatment with the anti-TSLP-R antibody, or antigen-binding fragment thereof. In some embodiments, the one or more symptom of CRSwNP is improved after, or about, 14 weeks of treatment with the anti-TSLP-R antibody, or antigen-binding fragment thereof. In some embodiments, the one or more symptom of CRSwNP is improved after, or about, 15 weeks of treatment with the anti-TSLP-R antibody, or antigen-binding fragment thereof. In some embodiments, the one or more symptom of CRSwNP is improved after, or about, 16 weeks of treatment with the anti-TSLP-R antibody, or antigen-binding fragment thereof. In some embodiments, the one or more symptom of CRSwNP is improved after, or about, 17 weeks of treatment with the anti-TSLP-R antibody, or antigen-binding fragment thereof. In some embodiments, the one or more symptom of CRSwNP is improved after, or about, 18 weeks of treatment with the anti-TSLP-R antibody, or antigen-binding fragment thereof. In some embodiments, the one or more symptom of CRSwNP is improved after, or about, 19 weeks of treatment with the anti-TSLP-R antibody, or antigen-binding fragment thereof. In some embodiments, the one or more symptom of CRSwNP is improved after, or about, 20 weeks of treatment with the anti-TSLP-R antibody, or antigen-binding fragment thereof. In some embodiments, the one or more symptom of CRSwNP is improved after, or about, 21 weeks of treatment with the anti-TSLP-R antibody, or antigen-binding fragment thereof. In some embodiments, the one or more symptom of CRSwNP is improved after, or about, 22 weeks of treatment with the anti-TSLP-R antibody, or antigen-binding fragment thereof. In some embodiments, the one or more symptom of CRSwNP is improved after, or about, 23 weeks of treatment with the anti-TSLP-R antibody, or antigen-binding fragment thereof. In some embodiments, the one or more symptom of CRSwNP is improved after, or about, 24 weeks of treatment with the anti-TSLP-R antibody, or antigen-binding fragment thereof.

In some embodiments, the one or more symptom of CRSwNP continues to improve after administration of the anti-TSLP-R antibody, or antigen-binding fragment thereof, has ceased for at least 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks.

In some embodiments, the method of treating CRSwNP comprises administering to the subject a therapeutically effective amount of the anti-TSLP-R antibody, or antigen-binding fragment thereof, wherein the subject achieves an improvement of CRSwNP symptoms following the treatment. In some embodiments, the method of treating CRSwNP comprises administering the anti-TSLP-R antibody, or antigen-binding fragment thereof, to a subject diagnosed with CRSwNP, wherein the subject achieves an improvement in one or more nasal polyp-associated parameters.

An “improvement in a nasal polyp-associated parameter” means an increase from baseline of one or more of NPIF, UPSIT, and/or a decrease from baseline of one or more of SNOT-22 score, subject-assessed nasal congestion/obstruction, anterior rhinorrhea (runny nose), posterior rhinorrhea (post nasal drip) and loss of sense of smell; number of nocturnal awakenings; VAS score; Lund-Mackay score; and 3D volumetric scores; and ACQ5 score in patients with asthma. As used herein, the term “baseline,” with regard to a nasal polyp-associated parameter, means the numerical value of the nasal polyp-associated parameter for a subject prior to or at the time of administration of the anti-TSLP-R antibody, or antigen-binding fragment thereof.

To determine whether a nasal polyp-associated parameter has “improved,” the parameter is quantified at baseline and at a time point after administration of the anti-TSLP-R antibody, or antigen-binding fragment thereof. For example, a nasal polyp-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the anti-TSLP-R antibody, or antigen-binding fragment thereof. In some embodiments, the parameter is measured daily (e.g., once or twice per day), weekly, biweekly, or monthly. In other embodiments, the parameter is measured daily and the mean value determined over the course of a month is compared to baseline.

The difference between the value of the parameter at a particular time point following the administration of the anti-TSLP-R antibody, or antibody fragment thereof and the value of the parameter at baseline is used to establish whether there has been an “improvement” in the nasal associated parameter (e.g., an increase or decrease).

In some embodiments, the method of treating CRSwNP comprises administering the anti-TSLP-R antibody, or antigen-binding fragment thereof, to a subject diagnosed with CRSwNP, wherein the subject achieves an improvement in one or more nasal polyp-associated parameters chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes.

In some embodiments, the method of treating CRSwNP comprises administering the anti-TSLP-R antibody, or antigen-binding fragment thereof, to a subject diagnosed with CRSwNP, wherein the subject achieves an improvement in endoscopic Nasal Polyp Score (NPS). A nasal polyp score (NPS) is a clinician-reported outcome measure scored after endoscopic evaluation of the nasal cavities. Each nostril is scored from 0 to 4. A NPS of 0 indicates the presence of no polyps. An NPS of 1 indicates the presence of small polyps in the middle meatus not reaching below the inferior border of the middle turbinate. An NPS of 2 indicates polyps reaching below lower border of middle turbinate. An NPS of 3 indicates large polyps reaching the lower border of the inferior turbinate or polyps medial to the middle turbinate. An NPS of 4 indicates large polyps causing complete obstruction of the inferior nasal cavity. Combined left and right scores give a total possible score range of 0-8, with higher scores indicating larger nasal polyps and greater disease severity.

In some embodiments, the method of treating CRSwNP comprises administering the anti-TSLP-R antibody, or antigen-binding fragment thereof, to a subject diagnosed with CRSwNP, wherein the subject achieves an improvement in endoscopic Nasal Polyp Score (NPS) by about 1 point, or a fraction thereof, by 2 points, or a fraction thereof, by 3 points, or a fraction thereof; by 4 points, or a fraction thereof, by 5 points, or a fraction thereof; by 6 points, or a fraction thereof; by 7 points, or a fraction thereof, or by 8 points or a fraction thereof, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the endoscopic Nasal Polyp Score by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30, about 31%, about 32%, about 33%, about 34%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%, as compared to baseline.

In some embodiments, the method of treating CRSwNP comprises administering the anti-TSLP-R antibody, or antigen-binding fragment thereof, to a subject diagnosed with CRSwNP, wherein the subject achieves an improvement in Nasal Congestion Score (NCS). NCS is a single-item PRO measure of whether a patient feels their nose is blocked. The score ranges from 0 (“not at all”) to 3 (“severe”) and is reported as average daily score over the previous 7 days.

In some embodiments, the method of treating CRSwNP comprises administering the anti-TSLP-R antibody, or antigen-binding fragment thereof, to a subject diagnosed with CRSwNP, wherein the subject achieves an improvement in Nasal Congestion Score (NCS) by about 1 point, or a fraction thereof; by 2 points, or a fraction thereof, or by 3 points, or a fraction thereof, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the Nasal Congestion Score (NCS) by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30, about 31%, about 32%, about 33%, about 34%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the Nasal Congestion Score (NCS) by at least −1.0, −1.5, or −1.75.

In some embodiments, the method of treating CRSwNP comprises administering the anti-TSLP-R antibody, or antigen-binding fragment thereof, to a subject diagnosed with CRSwNP, wherein the subject achieves an improvement in sinus opacification score (LMK score) by about 1 point, or a fraction thereof, by 2 points, or a fraction thereof, by 3 points, or a fraction thereof, by 4 points, or a fraction thereof, by 5 points, or a fraction thereof, by 6 points, or a fraction thereof, by 7 points, or a fraction thereof, by 8 points, or a fraction thereof, by 9 points, or a fraction thereof, or by 10 points, or a fraction thereof, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the sinus opacification score (LMK score) by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30, about 31%, about 32%, about 33%, about 34%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the sinus opacification score (LMK score) by at least −2, −3, −4, −5, −6, −7, −8, −9, or −10.

In some embodiments, the method of treating CRSwNP comprises administering the anti-TSLP-R antibody, or antigen-binding fragment thereof, to a subject diagnosed with CRSwNP, wherein the subject achieves an improvement in total symptom score (TSS) by about 1 point, or a fraction thereof; by 2 points, or a fraction thereof, by 3 points, or a fraction thereof, by 4 points, or a fraction thereof, by 5 points, or a fraction thereof, by 6 points, or a fraction thereof, by 7 points, or a fraction thereof, by 8 points, or a fraction thereof, by 9 points, or a fraction thereof, by 10 points, or a fraction thereof, or by 11 points, or a fraction thereof, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the total symptom score (TSS) by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30, about 31%, about 32%, about 33%, about 34%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the total symptom score (TSS) by at least −7, −8, −9, −10, or −11.

In some embodiments, the method of treating CRSwNP comprises administering the anti-TSLP-R antibody, or antigen-binding fragment thereof, to a subject diagnosed with CRSwNP, wherein the subject achieves an improvement in difficulty with smell score (DSS score) by about 1 point, or a fraction thereof, or by 2 points, or a fraction thereof, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the difficulty with smell score (DSS score) by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30, about 31%, about 32%, about 33%, about 34%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the difficulty with smell score (DSS score) by at least −1, −1.25, or −1.5.

In some embodiments, the method of treating CRSwNP comprises administering the anti-TSLP-R antibody, or antigen-binding fragment thereof, to a subject diagnosed with CRSwNP, wherein the subject achieves an improvement in nasal blockage score (NBS) by about 1 point, or a fraction thereof, or by 2 points, or a fraction thereof, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the nasal blockage score (NBS) by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30, about 31%, about 32%, about 33%, about 34%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100%, as compared to baseline. In some embodiments, the method of treating CRSwNP comprises administering the anti-TSLP-R antibody, or antigen-binding fragment thereof, to a subject diagnosed with CRSwNP, wherein the subject achieve an improvement in the nasal blockage score (NBS) by at least −1, −1.25, or −1.5.

In some embodiments, the method of treating CRSwNP comprises administering the anti-TSLP-R antibody, or antigen-binding fragment thereof, to a subject diagnosed with CRSwNP, wherein the subject achieves an improvement in the 22-Item Sinonasal Outcome Test (SNOT-22) Score. The SNOT-22 is a questionnaire to assess the impact of chronic rhinosinusitis (CRS) on quality of life. The questionnaire measures items related to sinonasal conditions and surgical treatments. The score ranges from 0 to 110, and higher scores imply greater impact of CRS on Health Related Quality of Life (HRQoL) (Hopkins et al 2009, Clin. Otolaryngol. 34: 447-454).

In some embodiments, the method of treating CRSwNP comprises administering the anti-TSLP-R antibody, or antigen-binding fragment thereof, to a subject diagnosed with CRSwNP, wherein the subject achieves a decrease in SNOT-22 score by about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the first administration of the anti-TSLP-R antibody, or antigen-binding fragment thereof following the administration of the anti-TSLP-R antibody, or antibody fragment thereof. In some embodiments, In some embodiments, the method of treating CRSwNP comprises administering the anti-TSLP-R antibody, or antigen-binding fragment thereof, to a subject diagnosed with CRSwNP, wherein the subject achieves a decrease in SNOT-22 score from baseline of about 1, 2, 3, 4, 5, 6, 7, 8, 9, 10, 11, 12, 13 points, or more score by about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the first administration of the anti-TSLP-R antibody, or antigen-binding fragment thereof.

In some embodiments, the method of treating CRSwNP comprises administering the anti-TSLP-R antibody, or antigen-binding fragment thereof, to a subject diagnosed with CRSwNP, wherein the subject achieves an improvement in subject-assessed symptoms. Subject-assessed symptoms are assayed by responding to morning and evening individual rhinosinusitis symptom questions using a 0-3 categorical scale (where 0=no symptoms, 1=mild symptoms, 2=moderate symptoms and 3=severe symptoms), and including the symptoms of congestion and/or obstruction, anterior rhinorrhea, posterior rhinorrhea, and loss of sense of smell. Nasal symptoms can be assayed in the day (AM), at night (PM) or both AM and PM.

In some embodiments, the method of treating CRSwNP comprises administering the anti-TSLP-R antibody, or antigen-binding fragment thereof, to a subject diagnosed with CRSwNP, wherein the subject achieves an improvement in one or more subject-assessed symptom chosen from nasal congestion/obstruction, anterior rhinorrhea (runny nose), posterior rhinorrhea (postnasal drip) and loss of sense of smell; and number of nocturnal awakenings.

In some embodiments, the method of treating CRSwNP comprises administering the anti-TSLP-R antibody, or antigen-binding fragment thereof, to a subject diagnosed with CRSwNP, wherein the subject achieves a decrease in loss of sense of smell (i.e., achieving a lower number on the scale) from baseline following the administration of the anti-TSLP-R antibody, or antibody fragment thereof. In some embodiments, the method of treating CRSwNP comprises administering the anti-TSLP-R antibody, or antigen-binding fragment thereof, to a subject diagnosed with CRSwNP, wherein the subject achieves a decrease in loss of sense of smell from baseline (e.g., from about 0.5, about 1.0, about 1.5, about 2.0, about 2.5 or about 3.0) that can be detected by about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves a decrease in congestion and/or obstruction (i.e., achieving a lower number on the scale) from baseline following the administration of the anti-TSLP-R antibody, or antibody fragment thereof. In some embodiments, the method of treating CRSwNP comprises administering the anti-TSLP-R antibody, or antigen-binding fragment thereof, to a subject diagnosed with CRSwNP, wherein the subject achieves a decrease in congestion and/or obstruction from baseline (e.g., from about 0.5, about 1.0, about 1.5, about 2.0, about 2.5 or about 3.0) that can be detected by about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves a decrease in runny nose (i.e., achieving a lower number on the scale) from baseline following the administration of the anti-TSLP-R antibody, or antibody fragment thereof. In some embodiments, the method of treating CRSwNP comprises administering the anti-TSLP-R antibody, or antigen-binding fragment thereof, to a subject diagnosed with CRSwNP, wherein the subject achieves a decrease in runny nose from baseline (e.g., from about 0.5, about 1.0, about 1.5, about 2.0, about 2.5 or about 3.0) that can be detected by about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves a decrease in post-nasal drip (i.e., achieving a lower number on the scale) from baseline following the administration of the anti-TSLP-R antibody, or antibody fragment thereof. In some embodiments, the method of treating CRSwNP comprises administering the anti-TSLP-R antibody, or antigen-binding fragment thereof, to a subject diagnosed with CRSwNP, wherein the subject achieves a decrease in post nasal drip from baseline (e.g., from about 0.5, about 1.0, about 1.5, about 2.0, about 2.5 or about 3.0) that can be detected by about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

Night-time awakenings can be assessed according to the following scores based on subject self-assessment: 0=no symptoms, slept through the night; 1=slept well, but some complaints in the morning; 2=woke up once because of rhinosinusitis symptoms (including early awakening); 3=woke up several times because of symptoms (including early awakening); 4=bad night, awake most of the night because of symptoms. In some embodiments, the method of treating CRSwNP comprises administering the anti-TSLP-R antibody, or antigen-binding fragment thereof, to a subject diagnosed with CRSwNP, wherein the subject achieves a decrease in average number of nighttime awakenings per night from baseline following the administration of the anti-TSLP-R antibody, or antibody fragment thereof. In some embodiments, the method of treating CRSwNP comprises administering the anti-TSLP-R antibody, or antigen-binding fragment thereof, to a subject diagnosed with CRSwNP, wherein the subject achieves a decrease in frequency of nighttime awakenings per night from baseline of at least about 0.10 times per night that can be detected by about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof. In some embodiments, the method of treating CRSwNP comprises administering the anti-TSLP-R antibody, or antigen-binding fragment thereof, to a subject diagnosed with CRSwNP, wherein the subject achieves a decrease in average number of nighttime awakenings per night from baseline by about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 time per night, 2.0 times per night, or more that can be detected by about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves an improvement in the Visual Analog Score (VAS) to assess patient-rated rhinosinusitis symptom severity. The VAS is a measure to assess patient-related rhinosinusitis symptom severity on a scale of 1 to 10. Mild symptoms are indicated by a score of 0 to 3, moderate symptoms are indicated by a VAS score of >3 to 7, and severe symptoms are indicated by a VAS score of >7 to 10. In some embodiments, the method of treating CRSwNP comprises administering the anti-TSLP-R antibody, or antigen-binding fragment thereof, to a subject diagnosed with CRSwNP, wherein the subject achieves a decrease in VAS score from baseline of about 0.5 point, 1 point, 1.5 points, 2 points, 2.5 points, 3 points, 3.5 points, 4 points, or more that can be detected by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves an improvement in the five-item Asthma Control Questionnaire (ACQ5) score. The ACQ5 measures both the adequacy of asthma control and change in asthma control, which occurs either spontaneously or as a result of treatment. The five questions on the ACQ5 reflect the top-scoring five asthma symptoms: woken at night by symptoms, wake in the mornings with symptoms, limitation of daily activities, shortness of breath and wheeze. Patients respond to the symptom questions on a 7-point scale (0=no impairment, totally controlled; 6=maximum impairment, severely uncontrolled). In some embodiments, the method of treating CRSwNP comprises administering the anti-TSLP-R antibody, or antigen-binding fragment thereof, to a subject diagnosed with CRSwNP, wherein the subject achieves a decrease in ACQ score from baseline of about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more that can be detected by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves an improvement in Nasal Peak Inspiratory Flow (NPIF). The Nasal Peak Inspiratory Flow (NPIF) represents a physiologic measure of air flow through both nasal cavities during forced inspiration and/or expiration expressed in liters per minute. Nasal inspiration correlates most with the subjective feeling of obstruction and is used to monitor nasal flow. In some embodiments, the method of treating CRSwNP comprises administering the anti-TSLP-R antibody, or antigen-binding fragment thereof, to a subject diagnosed with CRSwNP, wherein the subject achieves an increase in NPIF from baseline by about 0.10 liters per minute, 0.15 liters per minute, 0.20 liters per minute, 0.25 liters per minute, 0.30 liters per minute, 0.35 liters per minute, 0.40 liters per minute, 0.45 liters per minute, 0.50 liters per minute, 0.55 liters per minute, 0.60 liters per minute, 0.65 liters per minute, 0.70 liters per minute, 0.75 liters per minute, 0.80 liters per minute, 0.85 liters per minute, or more that can be detected by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves an improvement in the smell test (University of Pennsylvania Smell Identification Test (UPSIT)). The UPSIT is a method to quantitatively assess human olfactory function. The test consists of samples of odorants, and the subject has to describe the odor. The score is based on the number of correct answers. This test can distinguish patients with a normal sense of smell (“normosmia”) from those with different levels of reduction (“mild, moderate and severe microsmia”) or loss (“anosmia”). In some embodiments, the method of treating CRSwNP comprises administering the anti-TSLP-R antibody, or antigen-binding fragment thereof, to a subject diagnosed with CRSwNP, wherein the subject achieves an increase in UPSIT score from baseline by about 0.5 points, 1 point, 1.5 points, 2 points, 2.5 points, 3 points, 3.5 points or more that can be detected by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves an improvement in physiological parameters, such as lung function, capacity, and performance as measured by spirometry as performed according to the American Thoracic Society (ATS)/European Respiratory Society (ERS) spirometry guidelines. Spirometric measurements consist of determining the force expiratory volume in 1 second (FEV₁) as a percentage of the predicted normal value (PNV). Efficacy of treatment can be assayed by measuring the changes in FEV₁ from baseline. In some embodiments, the method of treating CRSwNP comprises administering the anti-TSLP-R antibody, or antigen-binding fragment thereof, to a subject diagnosed with CRSwNP, wherein the subject achieves an improvement in physiological parameters, including but not limited to lung function, capacity, and performance, that can be detected by spirometry as an increase in FEV₁ from baseline by about 1%, 2%, 3%, 4%, 5%, 6%, 7%, 8%, 9%, 10%, or more by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves an improvement in physiological parameters, such as measured by nasal endoscopy and CT scan. Efficacy of treatment be assayed by measuring the effect of physiological parameters, such as within the nasal cavities, such as by nasal endoscopy or computed tomography (CT) scan. In some embodiments, the method of treating CRSwNP comprises administering the anti-TSLP-R antibody, or antigen-binding fragment thereof, to a subject diagnosed with CRSwNP, wherein the subject achieves an improvement in physiological parameters that can be detected by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves an improvement in the Lund-Mackay Score. The Lund-Mackay scoring system is based on localization with points given for degree of opacification: 0=normal, 1=partial opacification, 2=total opacification. These points are then applied to the maxillary, anterior ethmoid, posterior ethmoid, sphenoid, and frontal sinus on each side. The osteomeatal complex is graded as 0=not occluded, or 2=occluded deriving a maximum score of 12 per side. For patients in whom the osteomeatal complex (OC) is missing (because of a previous surgery) the location of the former OC is considered and a score is provided, as if the OC was there. In some embodiments, the method of treating CRSwNP comprises administering the anti-TSLP-R antibody, or antigen-binding fragment thereof, to a subject diagnosed with CRSwNP, wherein the subject achieves a decrease in Lund-Mackay score from baseline by about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more that can be detected by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSwNP comprises administering the anti-TSLP-R antibody, or antigen-binding fragment thereof, to a subject diagnosed with CRSwNP, wherein the subject achieves an improvement in the Three Dimensional volumetric measurement of the maxillary sinus. The three-Dimensional Volumetric Measurement of Maxillary Sinus is used to calculate the volume of air (mL); the volume of mucosa (mL); the percent sinus occupied by disease; and the thickness of lateral wall in the maxillary sinus. In some embodiments, the method of treating CRSwNP comprises administering the anti-TSLP-R antibody, or antigen-binding fragment thereof, to a subject diagnosed with CRSwNP, wherein the subject achieves an increase in the Three-Dimensional volumetric measurement that can be detected by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the patient achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by that is an improvement to a score of 0 to 1 over time following administration of the anti-TSLP-R antibody, or antigen-binding fragment thereof.

In some embodiments, the method of treating CRSWNP comprises administering to a patient in need thereof a therapeutically effective amount of the anti-TSLP-R antibody, or antigen-binding fragment thereof, wherein the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, week 40, week 41, week 42, week 43, week 44, week 45, week 46, week 47, week 48, week 49, week 50, week 51, week 52, or over time following administration of the anti-TSLP-R antibody, or antigen-binding fragment thereof. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 1. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 2. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 3. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 4. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 5. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 6. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 7. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 8. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 9. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 10. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 11. In some embodiments, the subject achieves an improvement on an assessment chosen from the endoscopic Nasal Polyp Score (NPS); Sinus Opacification Score (LMK); Total Symptom Score (TSS); Difficulty With Smell Score (DSS); Nasal Blockage Score (NBS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 12. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 13. In some embodiments, the subject achieves an improvement on an assessment chosen from the endoscopic Nasal Polyp Score (NPS); Nasal Conestion Score (NCS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 14. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 15. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 16. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 17. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 18. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 19. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 20. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 21. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 22. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 23. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 24. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 25. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 26. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 27. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 28. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 29. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 30. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 31. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 32. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 33. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 34. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 35. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 36. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 37. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 38. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 39. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 40. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 41. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 42. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 43. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 44. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 45. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 46. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 47. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 48. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 49. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 50. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 51. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by week 52. In some embodiments, the subject achieves an improvement on an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by over time.

In some embodiments, the method of treating CRSWNP comprises administering to a patient in need thereof a therapeutically effective amount of the anti-TSLP-R antibody, or antigen-binding fragment thereof, wherein the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In some embodiments, the subject has sustained improvement of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by about 1 week after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 2 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 3 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 4 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 5 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 6 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 7 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 8 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 9 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 10 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 11 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 12 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 13 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 14 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 15 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 16 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 17 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 18 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 19 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 20 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for up to 52 weeks after treatment has ended.

In some embodiments, the method of treating CRSWNP comprises administering to a patient in need thereof a therapeutically effective amount of the anti-TSLP-R antibody, or antigen-binding fragment thereof, wherein the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In some embodiments, the subject has sustained improvement of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof by about 1 week after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 2 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 3 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 4 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 5 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 6 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 7 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 8 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 9 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 10 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 11 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 12 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 13 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 14 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 15 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 16 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 17 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 18 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 19 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for about 20 weeks after treatment has ended. In some embodiments, the subject does not experience worsening of one or more symptom assessed by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof for up to 52 weeks after treatment has ended.

In some embodiments, the method of treating CRSwNP comprises administering the anti-TSLP-R antibody, or antigen-binding fragment thereof, to a subject diagnosed with CRSwNP, wherein the subject achieves an improvement in patient reported outcomes. In some embodiments, the method of treating CRSwNP comprises administering the anti-TSLP-R antibody, or antigen-binding fragment thereof, to a subject diagnosed with CRSwNP, wherein the subject achieves an improvement in patient reported outcomes that can be detected by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

Non-limiting examples of assessments to measure patient reported outcomes include quality of life questionnaires including Short-Form-36 (SF-36) Questionnaire, the Eurogol-5D (EQ-5D), nasal polyp related resource use questionnaire, and the patient qualitative self-assessment.

The SF-36 is a 36 item questionnaire that measures eight multi-item dimensions of health: physical functioning (10 items) social functioning (2 items) role limitations due to physical problems (4 items), role limitations due to emotional problems (3 items), mental health (5 items), energy/vitality (4 items), pain (2 items), and general health perception (5 items). For each dimension, item scores are coded, summed, and transformed on a scale from 0 (worst possible health state measured by the questionnaire) to 100 (best possible health state). Two standardized summary scores can also be calculated from the SF-36; the physical component summary (PCS) and the mental health component summary (MCS).

The EQ-5D is a standardized health-related quality of life questionnaire developed by the EuroQoI Group in order to provide a simple, generic measure of health for clinical and economic appraisal and inter-disease comparisons. EQ-5D, designed for self-completion by patients, consists of two parts, the EQ-5D descriptive system and the EQ VAS. The EQ-5D descriptive system comprises 5 dimensions: mobility, self-care, usual activities, pain/discomfort and anxiety/depression; and each dimension has 3 levels: no problem, some problems, severe problems. The EQ Visual Analogue Scale (VAS) records the respondent's self-rated health on a vertical visual analogue scale. The EQ VAS ‘thermometer’ has endpoints of 100 (Best imaginable health state) at the top and 0 (Worst imaginable health state) at the bottom.

The nasal polyp related resource use questionnaire is a questionnaire of health care resource utilization for nasal polyposis, including specialist visits, emergency care visits, sick leaves, days off etc.

In some embodiments, the method of treating CRSwNP comprises administering the anti-TSLP-R antibody, or antigen-binding fragment thereof, to a subject diagnosed with CRSwNP, wherein the subject achieves an improvement in patient reported outcomes as measured by an assessment chosen from the Short-Form-36 (SF-36) Questionnaire, the Eurogol-5D (EQ-5D), nasal polyp related resource use questionnaire, or the patient qualitative self-assessment, or any combination thereof. In some embodiments, the method of treating CRSwNP comprises administering the anti-TSLP-R antibody, or antigen-binding fragment thereof, to a subject diagnosed with CRSwNP, wherein the subject achieves an improvement in patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof that can be detected by about 1 week, 2 week, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks following the administration of the anti-TSLP-R antibody, or antibody fragment thereof.

In some embodiments, the method of treating CRSWNP comprises administering to a patient in need thereof a therapeutically effective amount of the anti-TSLP-R antibody, or antigen-binding fragment thereof, wherein the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, week 40, week 41, week 42, week 43, week 44, week 45, week 46, week 47, week 48, week 49, week 50, week 51, week 52, or over time following administration of the anti-TSLP-R antibody, or antigen-binding fragment thereof. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 1. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 2. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 3. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 4. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 5. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 6. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 7. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 8. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 9. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 10. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 11. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 12. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 13. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 14. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 15. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 16. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 17. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 18. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 19. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 20. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 21. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 22. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 23. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 24. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 25. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 26. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 27. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 28. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 29. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 30. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 31. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 32. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 33. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 34. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 35. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 36. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 37. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 38. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 39. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 40. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 41. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 42. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 43. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 44. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 45. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 46. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 47. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 48. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 49. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 50. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 51. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, by week 52. In some embodiments, the subject achieves an improvement on patient reported outcomes as measured by an assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, over time.

In some embodiments, the method of treating CRSWNP comprises administering to a patient in need thereof a therapeutically effective amount of the anti-TSLP-R antibody, or antigen-binding fragment thereof, wherein the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In some embodiments, the subject has sustained improvement of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 1 week after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 2 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 3 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 4 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 5 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 6 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 7 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 8 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 9 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 10 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 11 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 12 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 13 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 14 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 15 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 16 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 17 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 18 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 19 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 20 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for up to 52 weeks after treatment has ended.

In some embodiments, the method of treating CRSWNP comprises administering to a patient in need thereof a therapeutically effective amount of the anti-TSLP-R antibody, or antigen-binding fragment thereof, wherein the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In some embodiments, the subject has sustained improvement of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 1 week after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 2 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 3 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 4 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 5 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 6 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 7 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 8 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 9 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 10 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 11 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 12 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 13 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 14 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 15 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 16 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 17 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 18 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 19 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for about 20 weeks after treatment has ended. In some embodiments, the subject does not experience worsening of one or more symptom assessed by the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof, for up to 52 weeks after treatment has ended.

In some embodiments, the method of treating CRSwNP comprises administering the anti-TSLP-R antibody, or antigen-binding fragment thereof, to a subject diagnosed with CRSwNP, wherein the subject achieves an improvement in a nasal polyp-associated biomarker.

Examples of nasal polyp-associated biomarkers include, but are not limited to, one or any combination of IgE, TARC, eotaxin-3 and ECP. In some embodiments, one or more nasal polyp-associated biomarkers may be detected from a biological sample derived from a subject. A biological sample includes, but is not limited to, one or any combination of materials taken from a patient including cultures, cells, tissues, blood, saliva, nasal secretions, cerebrospinal fluid, pleural fluid, milk, lymph, sputum, semen, needle aspirates, and the like. Biological samples may be obtained using any methods known in the art. For example, nasal secretion samples may be obtained from smears, blown secretions, imprints, lavage, swabs, brushes and the like.

A normal IgE level in healthy subjects is less than about 100 kU/L (e.g., as measured using the IMMUNOCAP® assay [Phadia, Inc. Portage, Mich.]). In some embodiments, the method of treating CRSwNP comprises administering the anti-TSLP-R antibody, or antigen-binding fragment thereof, to a subject diagnosed with CRSwNP, wherein the subject achieves a decrease in an elevated serum IgE level, which is a serum IgE level greater than about 100 kU/L, greater than about 150 kU/L, greater than about 500 kU/L, greater than about 1000 kU/L, greater than about 1500 kU/L, greater than about 2000 kU/L, greater than about 2500 kU/L, greater than about 3000 kU/L, greater than about 3500 kU/L, greater than about 4000 kU/L, greater than about 4500 kU/L, or greater than about 5000 kU/L following the administration of the anti-TSLP-R antibody, or antibody fragment thereof.

TARC levels in healthy subjects are in the range of 106 ng/L to 431 ng/L, with a mean of about 239 ng/L. (An exemplary assay system for measuring TARC level is the TARC quantitative ELISA kit offered as Cat. No. DDN00 by R&D Systems, Minneapolis, Minn.) In some embodiments, the method of treating CRSwNP comprises administering the anti-TSLP-R antibody, or antigen-binding fragment thereof, to a subject diagnosed with CRSwNP, wherein the subject achieves a decrease in an elevated serum TARC level, which is a serum TARC (e.g., serum TARC) level greater than about 431 ng/L, greater than about 500 ng/L, greater than about 1000 ng/L, greater than about 1500 ng/L, greater than about 2000 ng/L, greater than about 2500 ng/L, greater than about 3000 ng/L, greater than about 3500 ng/L, greater than about 4000 ng/L, greater than about 4500 ng/L, or greater than about 5000 ng/L following the administration of the anti-TSLP-R antibody, or antibody fragment thereof.

Improvement of a nasal polyp-associated parameter, such as a nasal polyp-associated parameter described above, can be expressed as a percentage. For example, a score can be improved by 30% or more, by 40% or more, by 50% or more, by 60% or more, by 70% or more, or by 80% or more.

Biomarker expression, as discussed above, can be assayed by detection of protein or RNA in serum. In some embodiments, RNA samples are used to determine RNA levels (non-genetic analysis), e.g., RNA levels of biomarkers; and in other embodiments, RNA samples are used for transcriptome sequencing (e.g., genetic analysis).

In some embodiments, the method of treating CRSWNP comprises administering to a patient in need thereof a therapeutically effective amount of the anti-TSLP-R antibody, or antigen-binding fragment thereof, wherein the subject achieves an improvement of a nasal polyp-associated biomarker chosen from IgE, TARC, eotaxin-3 and ECP, by week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, week 40, week 41, week 42, week 43, week 44, week 45, week 46, week 47, week 48, week 49, week 50, week 51, week 52, or over time following administration of the anti-TSLP-R antibody, or antigen-binding fragment thereof. In some embodiments, the subject achieves an improvement of a nasal polyp-associated biomarker chosen from IgE, TARC, eotaxin-3 and ECP, by week 1. In some embodiments, the subject achieves an improvement of a nasal polyp-associated biomarker chosen from IgE, TARC, eotaxin-3 and ECP, by week 2. In some embodiments, the subject achieves an improvement of a nasal polyp-associated biomarker chosen from IgE, TARC, eotaxin-3 and ECP, by week 3. In some embodiments, the subject achieves an improvement of a nasal polyp-associated biomarker chosen from IgE, TARC, eotaxin-3 and ECP, by week 4. In some embodiments, the subject achieves an improvement of a nasal polyp-associated biomarker chosen from IgE, TARC, eotaxin-3 and ECP, by week 5. In some embodiments, the subject achieves an improvement of a nasal polyp-associated biomarker chosen from IgE, TARC, eotaxin-3 and ECP, by week 6. In some embodiments, the subject achieves an improvement of a nasal polyp-associated biomarker chosen from IgE, TARC, eotaxin-3 and ECP, by week 7. In some embodiments, the subject achieves an improvement of a nasal polyp-associated biomarker chosen from IgE, TARC, eotaxin-3 and ECP, by week 8. In some embodiments, the subject achieves an improvement of a nasal polyp-associated biomarker chosen from IgE, TARC, eotaxin-3 and ECP, by week 9. In some embodiments, the subject achieves an improvement of a nasal polyp-associated biomarker chosen from IgE, TARC, eotaxin-3 and ECP, by week 10. In some embodiments, the subject achieves an improvement of a nasal polyp-associated biomarker chosen from IgE, TARC, eotaxin-3 and ECP, by week 11. In some embodiments, the subject achieves an improvement of a nasal polyp-associated biomarker chosen from IgE, TARC, eotaxin-3 and ECP, by week 12. In some embodiments, the subject achieves an improvement of a nasal polyp-associated biomarker chosen from IgE, TARC, eotaxin-3 and ECP, by week 13. In some embodiments, the subject achieves an improvement of a nasal polyp-associated biomarker chosen from IgE, TARC, eotaxin-3 and ECP, by week 14. In some embodiments, the subject achieves an improvement of a nasal polyp-associated biomarker chosen from IgE, TARC, eotaxin-3 and ECP, by week 15. In some embodiments, the subject achieves an improvement of a nasal polyp-associated biomarker chosen from IgE, TARC, eotaxin-3 and ECP, by week 16. In some embodiments, the subject achieves an improvement of a nasal polyp-associated biomarker chosen from IgE, TARC, eotaxin-3 and ECP, by week 17. In some embodiments, the subject achieves an improvement of a nasal polyp-associated biomarker chosen from IgE, TARC, eotaxin-3 and ECP, by week 18. In some embodiments, the subject achieves an improvement of a nasal polyp-associated biomarker chosen from IgE, TARC, eotaxin-3 and ECP, by week 19. In some embodiments, the subject achieves an improvement of a nasal polyp-associated biomarker chosen from IgE, TARC, eotaxin-3 and ECP, by week 20. In some embodiments, the subject achieves an improvement of a nasal polyp-associated biomarker chosen from IgE, TARC, eotaxin-3 and ECP, by week 21. In some embodiments, the subject achieves an improvement of a nasal polyp-associated biomarker chosen from IgE, TARC, eotaxin-3 and ECP, by week 22. In some embodiments, the subject achieves an improvement of a nasal polyp-associated biomarker chosen from IgE, TARC, eotaxin-3 and ECP, by week 23. In some embodiments, the subject achieves an improvement of a nasal polyp-associated biomarker chosen from IgE, TARC, eotaxin-3 and ECP, by week 24. In some embodiments, the subject achieves an improvement of a nasal polyp-associated biomarker chosen from IgE, TARC, eotaxin-3 and ECP, by week 25. In some embodiments, the subject achieves an improvement of a nasal polyp-associated biomarker chosen from IgE, TARC, eotaxin-3 and ECP, by week 26. In some embodiments, the subject achieves an improvement of a nasal polyp-associated biomarker chosen from IgE, TARC, eotaxin-3 and ECP, by week 27. In some embodiments, the subject achieves an improvement of a nasal polyp-associated biomarker chosen from IgE, TARC, eotaxin-3 and ECP, by week 28. In some embodiments, the subject achieves an improvement of a nasal polyp-associated biomarker chosen from IgE, TARC, eotaxin-3 and ECP, by week 29. In some embodiments, the subject achieves an improvement of a nasal polyp-associated biomarker chosen from IgE, TARC, eotaxin-3 and ECP, by week 30. In some embodiments, the subject achieves an improvement of a nasal polyp-associated biomarker chosen from IgE, TARC, eotaxin-3 and ECP, by week 31. In some embodiments, the subject achieves an improvement of a nasal polyp-associated biomarker chosen from IgE, TARC, eotaxin-3 and ECP, by week 32. In some embodiments, the subject achieves an improvement of a nasal polyp-associated biomarker chosen from IgE, TARC, eotaxin-3 and ECP, by week 33. In some embodiments, the subject achieves an improvement of a nasal polyp-associated biomarker chosen from IgE, TARC, eotaxin-3 and ECP, by week 34. In some embodiments, the subject achieves an improvement of a nasal polyp-associated biomarker chosen from IgE, TARC, eotaxin-3 and ECP, by week 35. In some embodiments, the subject achieves an improvement of a nasal polyp-associated biomarker chosen from IgE, TARC, eotaxin-3 and ECP, by week 36. In some embodiments, the subject achieves an improvement of a nasal polyp-associated biomarker chosen from IgE, TARC, eotaxin-3 and ECP, by week 37. In some embodiments, the subject achieves an improvement of a nasal polyp-associated biomarker chosen from IgE, TARC, eotaxin-3 and ECP, by week 38. In some embodiments, the subject achieves an improvement of a nasal polyp-associated biomarker chosen from IgE, TARC, eotaxin-3 and ECP, by week 39. In some embodiments, the subject achieves an improvement of a nasal polyp-associated biomarker chosen from IgE, TARC, eotaxin-3 and ECP, by week 40. In some embodiments, the subject achieves an improvement of a nasal polyp-associated biomarker chosen from IgE, TARC, eotaxin-3 and ECP, by week 41. In some embodiments, the subject achieves an improvement of a nasal polyp-associated biomarker chosen from IgE, TARC, eotaxin-3 and ECP, by week 42. In some embodiments, the subject achieves an improvement of a nasal polyp-associated biomarker chosen from IgE, TARC, eotaxin-3 and ECP, by week 43. In some embodiments, the subject achieves an improvement of a nasal polyp-associated biomarker chosen from IgE, TARC, eotaxin-3 and ECP, by week 44. In some embodiments, the subject achieves an improvement of a nasal polyp-associated biomarker chosen from IgE, TARC, eotaxin-3 and ECP, by week 45. In some embodiments, the subject achieves an improvement of a nasal polyp-associated biomarker chosen from IgE, TARC, eotaxin-3 and ECP, by week 46. In some embodiments, the subject achieves an improvement of a nasal polyp-associated biomarker chosen from IgE, TARC, eotaxin-3 and ECP, by week 47. In some embodiments, the subject achieves an improvement of a nasal polyp-associated biomarker chosen from IgE, TARC, eotaxin-3 and ECP, by week 48. In some embodiments, the subject achieves an improvement of a nasal polyp-associated biomarker chosen from IgE, TARC, eotaxin-3 and ECP, by week 49. In some embodiments, the subject achieves an improvement of a nasal polyp-associated biomarker chosen from IgE, TARC, eotaxin-3 and ECP, by week 50. In some embodiments, the subject achieves an improvement of a nasal polyp-associated biomarker chosen from IgE, TARC, eotaxin-3 and ECP, by week 51. In some embodiments, the subject achieves an improvement of a nasal polyp-associated biomarker chosen from IgE, TARC, eotaxin-3 and ECP, by week 52. In some embodiments, the subject achieves an improvement of a nasal polyp-associated biomarker chosen from IgE, TARC, eotaxin-3 and ECP, over time.

In some embodiments, the method of treating CRSWNP comprises administering to a patient in need thereof a therapeutically effective amount of the anti-TSLP-R antibody, or antigen-binding fragment thereof, wherein the subject does not experience worsening of a nasal polyp-associated biomarker chosen from IgE, TARC, eotaxin-3 and ECP, for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In some embodiments, the subject has sustained improvement of a nasal polyp-associated biomarker chosen from IgE, TARC, eotaxin-3 and ECP, for about 1 week after treatment has ended. In some embodiments, the subject does not experience worsening of a nasal polyp-associated biomarker chosen from IgE, TARC, eotaxin-3 and ECP, for about 2 weeks after treatment has ended. In some embodiments, the subject does not experience worsening of a nasal polyp-associated biomarker chosen from IgE, TARC, eotaxin-3 and ECP, for about 3 weeks after treatment has ended. In some embodiments, the subject does not experience worsening of a nasal polyp-associated biomarker chosen from IgE, TARC, eotaxin-3 and ECP, for about 4 weeks after treatment has ended. In some embodiments, the subject does not experience worsening of a nasal polyp-associated biomarker chosen from IgE, TARC, eotaxin-3 and ECP, for about 5 weeks after treatment has ended. In some embodiments, the subject does not experience worsening of a nasal polyp-associated biomarker chosen from IgE, TARC, eotaxin-3 and ECP, for about 6 weeks after treatment has ended. In some embodiments, the subject does not experience worsening of a nasal polyp-associated biomarker chosen from IgE, TARC, eotaxin-3 and ECP, for about 7 weeks after treatment has ended. In some embodiments, the subject does not experience worsening of a nasal polyp-associated biomarker chosen from IgE, TARC, eotaxin-3 and ECP, for about 8 weeks after treatment has ended. In some embodiments, the subject does not experience worsening of a nasal polyp-associated biomarker chosen from IgE, TARC, eotaxin-3 and ECP, for about 9 weeks after treatment has ended. In some embodiments, the subject does not experience worsening of a nasal polyp-associated biomarker chosen from IgE, TARC, eotaxin-3 and ECP, for about 10 weeks after treatment has ended. In some embodiments, the subject does not experience worsening of a nasal polyp-associated biomarker chosen from IgE, TARC, eotaxin-3 and ECP, for about 11 weeks after treatment has ended. In some embodiments, the subject does not experience worsening of a nasal polyp-associated biomarker chosen from IgE, TARC, eotaxin-3 and ECP, for about 12 weeks after treatment has ended. In some embodiments, the subject does not experience worsening of a nasal polyp-associated biomarker chosen from IgE, TARC, eotaxin-3 and ECP, for about 13 weeks after treatment has ended. In some embodiments, the subject does not experience worsening of a nasal polyp-associated biomarker chosen from IgE, TARC, eotaxin-3 and ECP, for about 14 weeks after treatment has ended. In some embodiments, the subject does not experience worsening of a nasal polyp-associated biomarker chosen from IgE, TARC, eotaxin-3 and ECP, for about 15 weeks after treatment has ended. In some embodiments, the subject does not experience worsening of a nasal polyp-associated biomarker chosen from IgE, TARC, eotaxin-3 and ECP, for about 16 weeks after treatment has ended. In some embodiments, the subject does not experience worsening of a nasal polyp-associated biomarker chosen from IgE, TARC, eotaxin-3 and ECP, for about 17 weeks after treatment has ended. In some embodiments, the subject does not experience worsening of a nasal polyp-associated biomarker chosen from IgE, TARC, eotaxin-3 and ECP, for about 18 weeks after treatment has ended. In some embodiments, the subject does not experience worsening of a nasal polyp-associated biomarker chosen from IgE, TARC, eotaxin-3 and ECP, for about 19 weeks after treatment has ended. In some embodiments, the subject does not experience worsening of a nasal polyp-associated biomarker chosen from IgE, TARC, eotaxin-3 and ECP, for about 20 weeks after treatment has ended. In some embodiments, the subject does not experience worsening of a nasal polyp-associated biomarker chosen from IgE, TARC, eotaxin-3 and ECP, for up to 52 weeks after treatment has ended.

In some embodiments, the method of treating CRSWNP comprises administering to a patient in need thereof a therapeutically effective amount of the anti-TSLP-R antibody, or antigen-binding fragment thereof, wherein the subject does not experience worsening of a nasal polyp-associated biomarker chosen from IgE, TARC, eotaxin-3 and ECP, for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended. In some embodiments, the subject has sustained improvement of a nasal polyp-associated biomarker chosen from IgE, TARC, eotaxin-3 and ECP, for about 1 week after treatment has ended. In some embodiments, the subject does not experience worsening of a nasal polyp-associated biomarker chosen from IgE, TARC, eotaxin-3 and ECP, for about 2 weeks after treatment has ended. In some embodiments, the subject does not experience worsening of a nasal polyp-associated biomarker chosen from IgE, TARC, eotaxin-3 and ECP, for about 3 weeks after treatment has ended. In some embodiments, the subject does not experience worsening of a nasal polyp-associated biomarker chosen from IgE, TARC, eotaxin-3 and ECP, for about 4 weeks after treatment has ended. In some embodiments, the subject does not experience worsening of a nasal polyp-associated biomarker chosen from IgE, TARC, eotaxin-3 and ECP, for about 5 weeks after treatment has ended. In some embodiments, the subject does not experience worsening of a nasal polyp-associated biomarker chosen from IgE, TARC, eotaxin-3 and ECP, for about 6 weeks after treatment has ended. In some embodiments, the subject does not experience worsening of a nasal polyp-associated biomarker chosen from IgE, TARC, eotaxin-3 and ECP, for about 7 weeks after treatment has ended. In some embodiments, the subject does not experience worsening of a nasal polyp-associated biomarker chosen from IgE, TARC, eotaxin-3 and ECP, for about 8 weeks after treatment has ended. In some embodiments, the subject does not experience worsening of a nasal polyp-associated biomarker chosen from IgE, TARC, eotaxin-3 and ECP, for about 9 weeks after treatment has ended. In some embodiments, the subject does not experience worsening of a nasal polyp-associated biomarker chosen from IgE, TARC, eotaxin-3 and ECP, for about 10 weeks after treatment has ended. In some embodiments, the subject does not experience worsening of a nasal polyp-associated biomarker chosen from IgE, TARC, eotaxin-3 and ECP, for about 11 weeks after treatment has ended. In some embodiments, the subject does not experience worsening of a nasal polyp-associated biomarker chosen from IgE, TARC, eotaxin-3 and ECP, for about 12 weeks after treatment has ended. In some embodiments, the subject does not experience worsening of a nasal polyp-associated biomarker chosen from IgE, TARC, eotaxin-3 and ECP, for about 13 weeks after treatment has ended. In some embodiments, the subject does not experience worsening of a nasal polyp-associated biomarker chosen from IgE, TARC, eotaxin-3 and ECP, for about 14 weeks after treatment has ended. In some embodiments, the subject does not experience worsening of a nasal polyp-associated biomarker chosen from IgE, TARC, eotaxin-3 and ECP, for about 15 weeks after treatment has ended. In some embodiments, the subject does not experience worsening of a nasal polyp-associated biomarker chosen from IgE, TARC, eotaxin-3 and ECP, for about 16 weeks after treatment has ended. In some embodiments, the subject does not experience worsening of a nasal polyp-associated biomarker chosen from IgE, TARC, eotaxin-3 and ECP, for about 17 weeks after treatment has ended. In some embodiments, the subject does not experience worsening of a nasal polyp-associated biomarker chosen from IgE, TARC, eotaxin-3 and ECP, for about 18 weeks after treatment has ended. In some embodiments, the subject does not experience worsening of a nasal polyp-associated biomarker chosen from IgE, TARC, eotaxin-3 and ECP, for about 19 weeks after treatment has ended. In some embodiments, the subject does not experience worsening of a nasal polyp-associated biomarker chosen from IgE, TARC, eotaxin-3 and ECP, for about 20 weeks after treatment has ended. In some embodiments, the subject does not experience worsening of a nasal polyp-associated biomarker chosen from IgE, TARC, eotaxin-3 and ECP, for up to 52 weeks after treatment has ended.

In some embodiments, the method of treating CRSWNP comprises administering to a patient in need thereof a therapeutically effective amount of the anti-TSLP-R antibody, or antigen-binding fragment thereof, wherein the subject achieves an improvement in a symptom of CRSwNP as measured by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or any combination thereof.

In some embodiments, the method of treating CRSWNP comprises administering to a patient in need thereof a therapeutically effective amount of the anti-TSLP-R antibody, or antigen-binding fragment thereof, wherein the subject's assessment of patient reported outcomes is improved after treatment. In some embodiments, the method of treating CRSWNP comprises administering to a patient in need thereof a therapeutically effective amount of the anti-TSLP-R antibody, or antigen-binding fragment thereof, wherein the subject's assessment of patient reported outcomes is improved according to one or more assessment chosen from the Short-Form-36 (SF-36) Questionnaire, the Eurogol-5D (EQ-5D), nasal polyp related resource use questionnaire, the patient qualitative self-assessment, and other assessments of patient reported outcomes, or any combination thereof.

In some embodiments, the method of treating CRSWNP comprises administering to a patient in need thereof a therapeutically effective amount of the anti-TSLP-R antibody, or antigen-binding fragment thereof, wherein the subject achieves an improvement of a nasal polyp-associated biomarker, compared to baseline. In some embodiments, the method of treating CRSWNP comprises administering to a patient in need thereof a therapeutically effective amount of the anti-TSLP-R antibody, or antigen-binding fragment thereof, wherein the subject achieves an improvement of a nasal polyp-associated biomarker chosen from IgE, TARC, eotaxin-3 and ECP, compared to baseline.

In some embodiments, the method of treating CRSwNP comprises administering to a patient in need thereof a therapeutically effective amount of the anti-TSLP-R antibody, or antigen-binding fragment thereof, wherein the method may produce long lasting effects on the nasal tissue and may modify the long-term course of CRSwNP. Specifically, in some embodiments, the improvement of the symptoms may be maintained long after the final administration of the anti-TSLP-R antibody, or antigen-binding fragment thereof, as described herein. In some embodiments, the improvement continues over time after administration of the anti-TSLP-R antibody, or antigen-binding fragment thereof, has ceased. In some embodiments, the improvement continues for greater than or equal to 1 month, 2 months, 3 months, 4 months, 5 months, 6 months, 7 months, 8 months, 9 months, 10 months, 11 months, or 12 months after administration of the anti-TSLP-R antibody, or antigen-binding fragment thereof, has ceased.

Doses may be provided, e.g., intravenously, subcutaneously, or intramuscularly. In some embodiments, the doses are provided intravenously. In some embodiments, the doses are provided subcutaneously. The pharmaceutical compositions provided herein can be provided by continuous infusion, or by doses administered, e.g., monthly, bimonthly, quarterly, semiannually, etc. Doses may be provided, e.g., intravenously or subcutaneously. In some embodiments, the pharmaceutical composition is administered (e.g., intravenous or subcutaneous injection) once. In some embodiments, the pharmaceutical composition is administered (e.g., intravenous or subcutaneous injection) twice. In some embodiments, the pharmaceutical composition is administered (e.g., intravenous or subcutaneous injection) three times. In some embodiments, the pharmaceutical composition is administered (e.g., intravenous or subcutaneous injection) four times. In some embodiments, the pharmaceutical composition is administered (e.g., intravenous or subcutaneous injection) more than four times. When the pharmaceutical composition is administered more than once it can be administered according to a schedule, such as the schedules provided for herein.

In some embodiments, a method of treating CRSwNP in a subject in need thereof is provided, the method comprising administering to the subject about 1.0 mg/kg to about 15.0 mg/kg of an anti-TSLP-R antibody, or antigen-binding fragment thereof, wherein the anti-TSLP-R antibody, or antigen-binding fragment thereof, comprises: (i) a heavy chain having a sequence of SEQ ID NO: 1; and (ii) a light chain having a sequence of SEQ ID NO: 3, wherein the subject achieves an improvement in one or more symptom of CRSwNP chosen from loss of smell, nasal congestion, anterior nasal discharge, posterior nasal discharge, nasal peak inspiratory flow (NPIF) that occur in the day (AM), nasal peak inspiratory flow (NPIF) that occur in the night (PM), facial pain, headache, or any combination thereof.

In some embodiments, a method of treating CRSwNP in a subject in need thereof is provided, the method comprising administering to the subject about 1.0 mg/kg to about 15.0 mg/kg of an anti-TSLP-R antibody, or antigen-binding fragment thereof, wherein the anti-TSLP-R antibody, or antigen-binding fragment thereof, comprises: (i) a heavy chain having a sequence of SEQ ID NO: 1; and (ii) a light chain having a sequence of SEQ ID NO: 3, wherein the subject achieves an improvement of one or more symptom of CRSwNP as measured by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, patient reported outcomes, or any combination thereof, compared to baseline.

In some embodiments, a method of treating CRSwNP in a subject in need thereof is provided, the method comprising administering to the subject about 1.0 mg/kg to about 15.0 mg/kg of an anti-TSLP-R antibody, or antigen-binding fragment thereof, wherein the anti-TSLP-R antibody, or antigen-binding fragment thereof, comprises: (i) a heavy chain having a sequence of SEQ ID NO: 1; and (ii) a light chain having a sequence of SEQ ID NO: 3, wherein the subject achieves an improvement of one or more patient reported outcomes according to one or more assessment chosen from the Short-Form-36 (SF-36) Questionnaire, the Eurogol-5D (EQ-5D), nasal polyp related resource use questionnaire, the patient qualitative self-assessment, or any combination thereof, compared to baseline.

In some embodiments, a method of treating CRSwNP in a subject in need thereof is provided, the method comprising administering to the subject about 1.0 mg/kg to about 15.0 mg/kg of an anti-TSLP-R antibody, or antigen-binding fragment thereof, wherein the anti-TSLP-R antibody, or antigen-binding fragment thereof, comprises: (i) a heavy chain having a sequence of SEQ ID NO: 1; and (ii) a light chain having a sequence of SEQ ID NO: 3, wherein the subject achieves an improvement of a nasal polyp-associated biomarker, compared to baseline.

In some embodiments, a method of treating CRSwNP in a subject in need thereof is provided, the method comprising administering to the subject about 1.0 mg/kg to about 15.0 mg/kg of an anti-TSLP-R antibody, or antigen-binding fragment thereof, wherein the anti-TSLP-R antibody, or antigen-binding fragment thereof, comprises: (i) a heavy chain having a sequence of SEQ ID NO: 1; and (ii) a light chain having a sequence of SEQ ID NO: 3, wherein the subject achieves an improvement of a nasal polyp-associated biomarker chosen from IgE, TARC, eotaxin-3 and ECP, compared to baseline.

In some embodiments, a method of treating CRSwNP in a subject in need thereof is provided, the method comprising administering to the subject about 1.0 mg/kg to about 15.0 mg/kg of an anti-TSLP-R antibody, or antigen-binding fragment thereof, wherein the anti-TSLP-R antibody, or antigen-binding fragment thereof, comprises: (i) a heavy chain having a sequence of SEQ ID NO: 1; and (ii) a light chain having a sequence of SEQ ID NO: 3, wherein the subject achieves a reduction in the size of one or more nasal polyp as assessed by the nasal polyp score (NPS) compared to baseline.

In some embodiments, a method of treating CRSwNP in a subject in need thereof is provided, the method comprising administering to the subject about 1.0 mg/kg to about 15.0 mg/kg of an anti-TSLP-R antibody, or antigen-binding fragment thereof, wherein the anti-TSLP-R antibody, or antigen-binding fragment thereof, comprises: (i) a heavy chain having a sequence of SEQ ID NO: 1; and (ii) a light chain having a sequence of SEQ ID NO: 3, wherein the subject achieves a reduction in the number of nasal polyps present compared to baseline.

In some embodiments, a method of treating CRSwNP in a subject in need thereof is provided, the method comprising administering to the subject about 1.0 mg/kg to about 15.0 mg/kg of an anti-TSLP-R antibody, or antigen-binding fragment thereof, wherein the anti-TSLP-R antibody, or antigen-binding fragment thereof, comprises: (i) a heavy chain having a sequence of SEQ ID NO: 1; and (ii) a light chain having a sequence of SEQ ID NO: 3, wherein the need for treatment with intranasal or oral corticosteroids is reduced in the subject.

In some embodiments, a method of treating CRSwNP in a subject in need thereof is provided, the method comprising administering to the subject about 1.0 mg/kg to about 15.0 mg/kg of an anti-TSLP-R antibody, or antigen-binding fragment thereof, wherein the anti-TSLP-R antibody, or antigen-binding fragment thereof, comprises: (i) a heavy chain having a sequence of SEQ ID NO: 1; and (ii) a light chain having a sequence of SEQ ID NO: 3, wherein the need for treatment with nasal surgery is reduced in the subject.

In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a blood eosinophil concentration of at least 200 eosinophiles/μL, 300 eosinophiles/μL, or 400 eosinophiles/L prior to the administration of the anti-TSLP-R antibody, or antigen-binding fragment thereof. In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a blood eosinophil concentration of at least 200 eosinophiles/L prior to the administration of the anti-TSLP-R antibody, or antigen-binding fragment thereof. In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a blood eosinophil concentration of at least 300 eosinophiles/μL prior to the administration of the anti-TSLP-R antibody, or antigen-binding fragment thereof. In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a blood eosinophil concentration of at least 400 eosinophiles/L prior to the administration of the anti-TSLP-R antibody, or antigen-binding fragment thereof.

In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a decrease in blood eosinophil concentration following the administration of the anti-TSLP-R antibody, or antigen-binding fragment thereof. In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a continued decrease in blood eosinophil concentration after administration of anti-TSLP-R antibody, or antigen-binding fragment thereof, has ceased. In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a continued decrease in blood eosinophil concentration after at least 1 administration of the anti-TSLP-R antibody, or antigen-binding fragment thereof. In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a continued decrease in blood eosinophil concentration for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, or about 24 weeks after the at least 1 administration of the anti-TSLP-R antibody, or antigen-binding fragment thereof.

In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the pharmaceutical composition, or the pharmaceutical dosage is administered to the subject about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, about every 20 weeks, about every 21 weeks, about every 22 weeks, about every 23 weeks, or about every 24 weeks; and the subject has a decrease in blood eosinophil concentration following 2, 3, 4, 5, or more administrations of the anti-TSLP-R antibody, or antigen-binding fragment thereof. In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the pharmaceutical composition, or the pharmaceutical dosage is administered to the subject every 4 weeks; and the subject has a decrease in blood eosinophil concentration following 3 administrations of the anti-TSLP-R antibody, or antigen-binding fragment thereof.

In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a decrease in blood eosinophil concentration that is sustained after administration of the anti-TSLP-R antibody, or antigen-binding fragment thereof has ceased for at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks.

In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the subject has a sustained and measurable serum concentration of the anti-TSLP-R antibody, or antigen-binding fragment thereof after administration has ceased for at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks. In some embodiments, the method of treating Chronic Rhinosinusitis with Nasal Polyps in a subject in need thereof comprises administering to the subject a therapeutically effective amount of the anti-TSLP-R antibody, or antigen-binding fragment thereof, as described herein, wherein the anti-TSLP-R antibody, or antigen-binding fragment thereof, is administered to the subject about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, about every 20 weeks, about every 21 weeks, about every 22 weeks, about every 23 weeks, or about every 24 weeks; at least 2 administrations or at least 3 administrations of the anti-TSLP-R antibody, or antigen-binding fragment thereof, are administered; and the subject has a sustained and measurable serum concentration of the anti-TSLP-R antibody, or antigen-binding fragment thereof after administration has ceased for at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks.

In some embodiments, a method of treating chronic rhinosinusitis with nasal polyps (CRSwNP) in a subject in need thereof comprises administering a pharmaceutical composition comprising about 100 to about 150 mg of verekitug to the subject in need thereof. In some embodiments, any anti-TSLP-R antibody binding to the same epitope on TSLP-R as verekitug and with the same binding affinity as verekitug may be used. In some embodiments, a method of treating chronic rhinosinusitis with nasal polyps (CRSwNP) in a subject in need thereof comprises administering a pharmaceutical composition comprising about 100 to about 150 mg of an anti-TSLP-R antibody binding to the same epitope on TSLP-R as verekitug and with the same binding affinity as verekitug, to the subject in need thereof. In some embodiments, the composition is administered once every twelve weeks. In some embodiments, the pharmaceutical composition comprises about 100, about 125, or about 150 mg of verekitug. In some embodiments, the pharmaceutical composition is administered subcutaneously. In some embodiments, verekitug binds to the TSLP receptor. In some embodiments, verekitug blocks the formation of the heterodimeric TSLP receptor. In some embodiments, the subject has a history of nasal polyp (NP) surgery or NP exacerbation requiring steroids (e.g. corticosteroids) being administered to the subject sometime in the 24 months preceding the administration of the pharmaceutical composition. In some embodiments, the subject has a endoscopic nasal polyp score (NPS)≥5. In some embodiments, the subject has a nasal congestion score (NCS)≥2 over 14 days. In some embodiments, the subject has CRSwNP symptoms for at least 8 weeks. In some embodiments, the subject in need thereof has had stable standard of care for the treatment of CRSwNP for at least 30 days. In some embodiments, the subject in need thereof has asthma. In some embodiments, the subject is administered a second dose 12 weeks after the first dose. In some embodiments, the subject achieves an improvement in at least one of: a nasal polyp score (NPS), a nasal congestion score (NCS), a sinus opacification score (LMK score), a total symptom score (TSS), a difficulty with smell score (DSS score), or a nasal blockage score (NBS). In some embodiments, the reduction in the nasal polyp score (NPS) is at least −1.0, −1.5, −2.0, or −2.5. In some embodiments, the reduction in the nasal polyp score (NPS) is measured at 12 weeks from the first dose. In some embodiments, the reduction in the nasal polyp score (NPS) is measured at 24 weeks from the first dose. In some embodiments, the subject achieves the improvement in the nasal congestion score (NCS) at 2 weeks, at 3 weeks, at 4 weeks, at 5 weeks, or at 6 weeks after administration of the first dose. In some embodiments, the change of the nasal congestion score (NCS) is a reduction by at least −1.0, −1.5, or −1.75. In some embodiments, the reduction in the nasal congestion score (NCS) is measured at 24 weeks from the first dose. In some embodiments, the sinus opacification (LMK score) change from baseline is at least −2, −3, −4, −5, −6, −7, −8, −9, or −10. In some embodiments, the sinus opacification (LMK score) change from baseline is measured at 24 weeks from the first dose. In some embodiments, the total symptom score (TSS) change from baseline is at least −7, −8, −9, −10, or −11. In some embodiments, the TSS change from baseline is measured at 24 weeks from the first dose. In some embodiments, the difficulty with smell (DSS score) change from baseline is at least −1, −1.25, or −1.5. In some embodiments, the nasal blockage score (NBS) change from baseline is at least −1, −1.25, or −1.5. In some embodiments, the subject in need thereof treated is less likely to require sinus surgery to treat the polyps.

In some embodiments, provided herein is a use of verekitug for the manufacture of a medicament for treating chronic rhinosinusitis with nasal polyps (CRSwNP) in a subject in need thereof, wherein the medicament comprises about 100 to about 150 mg of verekitug.

### Kits

Kits are also provided which are useful for carrying out the methods described herein. In some embodiments, a kit is provided, comprising any of the pharmaceutical compositions, as disclosed herein, and instructions for use. In some embodiments, a kit is provided, comprising any of the pharmaceutical compositions, as disclosed herein, in a container, and instructions for use. In some embodiments, a kit is provided, comprising any of the pharmaceutical compositions, as disclosed herein, in a container, wherein the container is a pre-filled syringe, and instructions for use. In some embodiments, a kit is provided, comprising any of the pharmaceutical compositions, as disclosed herein, in a container, wherein the container is a plastic vial, or glass vial, and instructions for use.

The kit includes a container holding antibodies, or antigen-binding fragments thereof, which binds an epitope of TSLP-R protein and instructions for using the antibodies, or antigen-binding fragments thereof, for the purpose of binding to TSLP-R protein to form an immunological complex and detecting the formation of the immunological complex such that the presence or absence of the immunological complex correlates with presence or absence of TSLP-R protein in the sample.

The present kits comprise a first container containing or packaged in association with the above-described antibodies, or fragments thereof. The kit may also comprise another container containing or packaged in association solutions necessary or convenient for carrying out the methods disclosed herein. The containers can be made of glass, plastic or foil and can be a vial, bottle, pouch, tube, bag, etc. The kit may also contain written information, such as procedures for carrying out the methods disclosed herein, or analytical information, such as the amount of reagent contained in the first container. The container may be in another container apparatus, e.g., a box or a bag, along with the written information. Examples of containers include multiwell plates which allow simultaneous detection of TSLP-R protein in multiple samples.

Yet another aspect provided for herein is a kit for detecting TSLP-R protein in a biological sample. The kit includes a container holding antibodies, or fragments thereof, which binds an epitope of TSLP-R protein and instructions for using the antibodies, or fragments thereof, for the purpose of binding to TSLP-R protein to form an immunological complex and detecting the formation of the immunological complex such that the presence or absence of the immunological complex correlates with presence or absence of TSLP-R protein in the sample. Examples of containers include multi-well plates which allow simultaneous detection of TSLP-R protein in multiple samples.

In some embodiments, the kit comprises antibodies, or fragments thereof, that bind to a TSLP-R protein. In some embodiments, the anti-TSLP-R antibody, or antigen-binding fragment thereof, is isolated. In some embodiments, the anti-TSLP-R antibody, or antigen-binding fragment thereof, binds to a TSLP-R protein that is properly folded. In some embodiments, the anti-TSLP-R antibody, or antigen-binding fragment thereof, binds to a TSLP-R protein in a cell membrane. In some embodiments, the anti-TSLP-R antibody, or antigen-binding fragment thereof, binds to a TSLP-R protein that is in a cell membrane in an intact cell. In some embodiments, the anti-TSLP-R antibody, or antigen-binding fragment thereof, inhibits or neutralizes the function of a TSLP-R protein. As used herein, the term “neutralize” means that the activity or function of the protein is inhibited. The inhibition can be complete or partial. In some embodiments, the activity or function of the protein is inhibited at least 10%, 20%, 30%, 40%, 50%, 60%, 70%, 80%, 90%, 95%, or 99%. The percent inhibition can be based upon the function or activity of the protein in the absence of the antibody, or fragment thereof. In some embodiments, the anti-TSLP-R antibody, or antigen-binding fragment thereof, inhibits the internalization of the TSLP-R protein.

The antibodies, or fragments thereof, can be provided in a kit, such as those provided herein. The antibodies, or fragments thereof, can be used or administered alone or in admixture with another therapeutic, analgesic, or diagnostic agent, such as provided for herein. In some embodiments, the methods comprise administering an antibody, or fragment thereof, such as those provided herein.

Although the present disclosure has been described in considerable detail with reference to certain preferred embodiments thereof, other versions are possible. Therefore, the spirit and scope of the appended claims should not be limited to the description and the preferred versions contained within this specification.

### EXAMPLES

Various aspects of the present disclosure are illustrated with reference to the following non-limiting examples.

### Example 1: In Vitro Studies

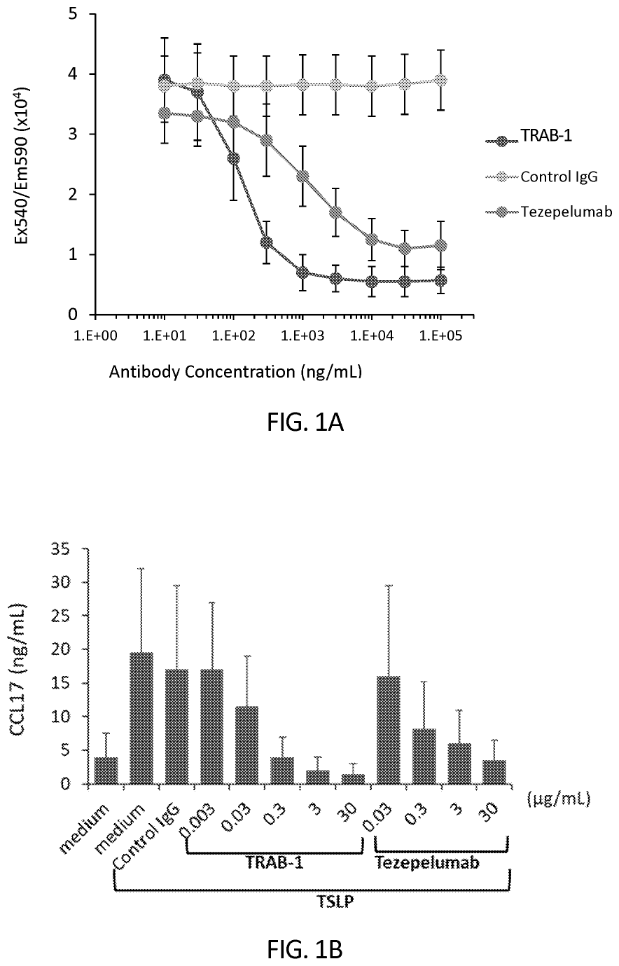

Ba/F3 cells were transfected with human TSLP-R/IL-7Rα and treated with either TRAB-1 antibody (the anti-TSLP-R antibody described herein), tezepelumab antibody (an anti-TSLP antibody), or IgG antibody. Cell proliferation rates in the Ba/F3 cells were monitored as a function of the concentration of the antibody concentration (FIG. 1A). IC50 and IC90 values are plotted in Table 5.

TABLE 5
IC50 and IC90 values from Ba/F3 cells transfected
with human TSLP-R/IL-7Rα and treated with
either TRAB-1 antibody or tezepelumab antibody.

| | IC50 (ng/mL) | IC90 (ng/ml) |
|---|---|---|
| TRAB-1 | 90.7 | 200 |
| Tezepelumab | 518 | 15300 |

Dendritic cells were treated with TRAB-1 antibody, tezepelumab antibody, or IgG antibody. The CCL17 (ng/mL) production in the dendritic cells was monitored as a function of the concentration of the antibody concentration (FIG. 1). IC50 and IC90 values are plotted in Table 6.

TABLE 6
IC50 and IC90 values from dendritic cells treated with
either TRAB-1 antibody or tezepelumab antibody

| | IC50 (ng/ml) | IC90 (ng/ml) |
|---|---|---|
| TRAB-1 | 16.1 | 163 |
| Tezepelumab | 67.0 | 855 |

IC90 values from these in vitro assays suggested a trough concentration of approximately 0.3 ng/mL. Together, these data depict that TRAB-1 produces potent suppression of TSLP/TSLP-R-mediated responses in vitro.

### Example 2: Single Ascending Dose Studies in Healthy Volunteers

Single ascending dose (SAD) studies were performed in healthy volunteers. Healthy volunteer subjects (n=36) were treated with 0.03-10 mg/kg single doses of TRAB-1 either intravenously (IV) or subcutaneously (SC). The doses chosen for study were: 0.03 mg/kg IV; 0.1 mg/kg IV; 0.3 mg/kg IV; 1.0 mg/kg IV; 3 mg/kg IV; 10 mg/kg IV; and 1.0 mg/kg SC. No serious aversive events were observed in the group of healthy volunteers. The most common emergent adverse event in the treatment group was: dysmenorrhea (3, 8.3%), breast tenderness (3, 8.3%), and headache (1, 2.8%).

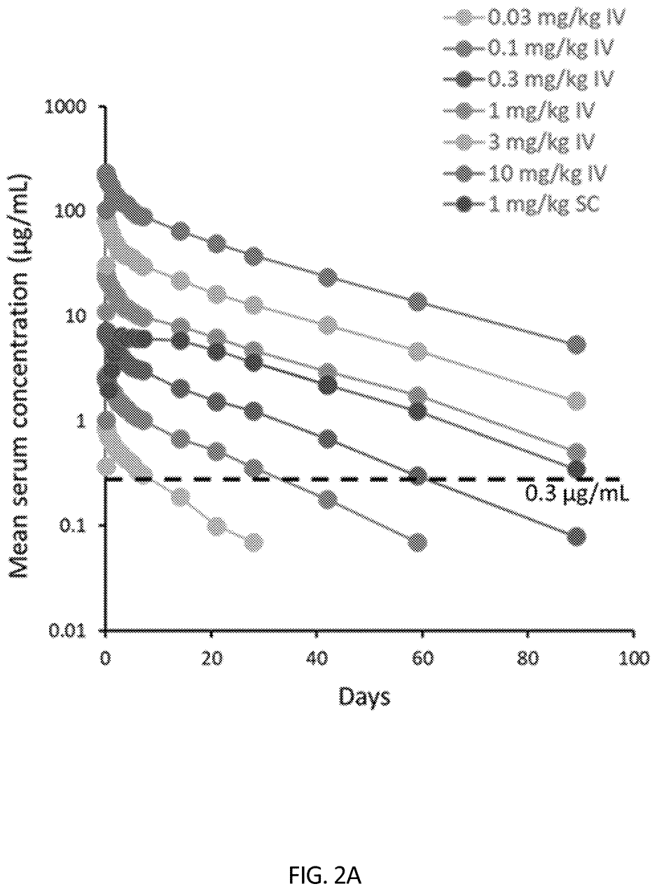

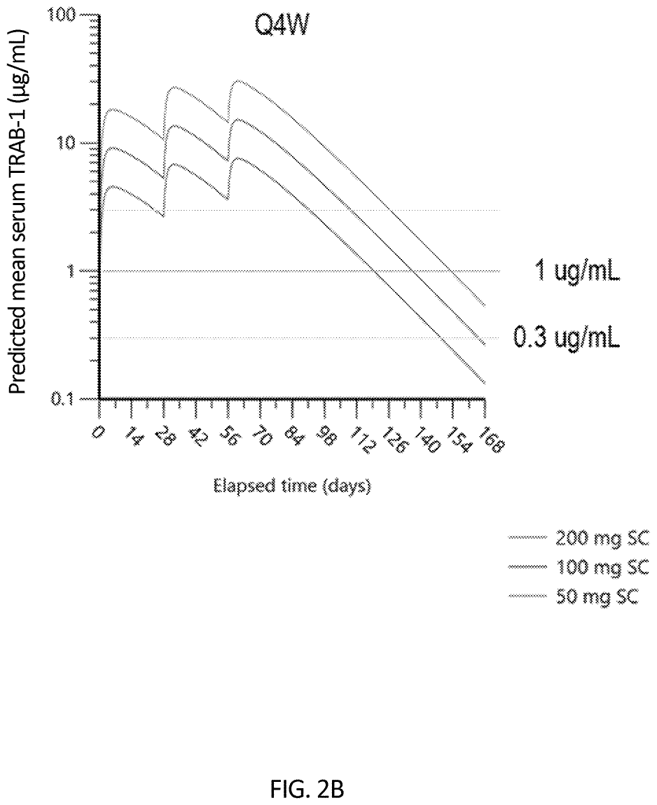

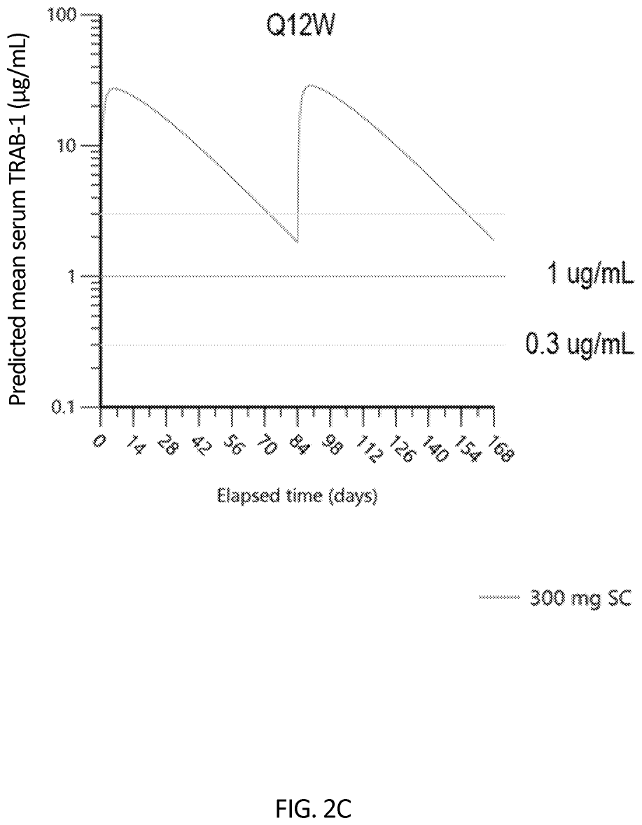

Mean serum concentration as a function of dose and route of administration of TRAB-1 was monitored over time (FIG. 2A). This data demonstrated sustained pharmacokinetics (PK) effect over 60 days. Dose-proportional pharmacokinetics were observed from 2 mg TRAB-1 to 700 mg TRAB-1. About 70% bioavailability was observed with sub-cutaneous administration. Doses of greater than or equal to 1.0 mg/kg resulted in exposure greater than or equal to the therapeutic target (0.3 g/mL). Lower titer antidrug-antibodies (ADAs) were observed in 24% of subjects, with minimal impact on pharmacokinetics. The following dosing schedules were chosen for further study: every four weeks (Q4W), and every 12 weeks (Q12W). Predicted mean serum concentration of TRAB-1 overtime, as a function of dose of TRAB-1 administered subcutaneously every 4 weeks, is shown in FIG. 2B. Predicted mean serum concentration of TRAB-1 over time, as a function of dose of TRAB-1 administered subcutaneously every 12 weeks, is shown in FIG. 2C.

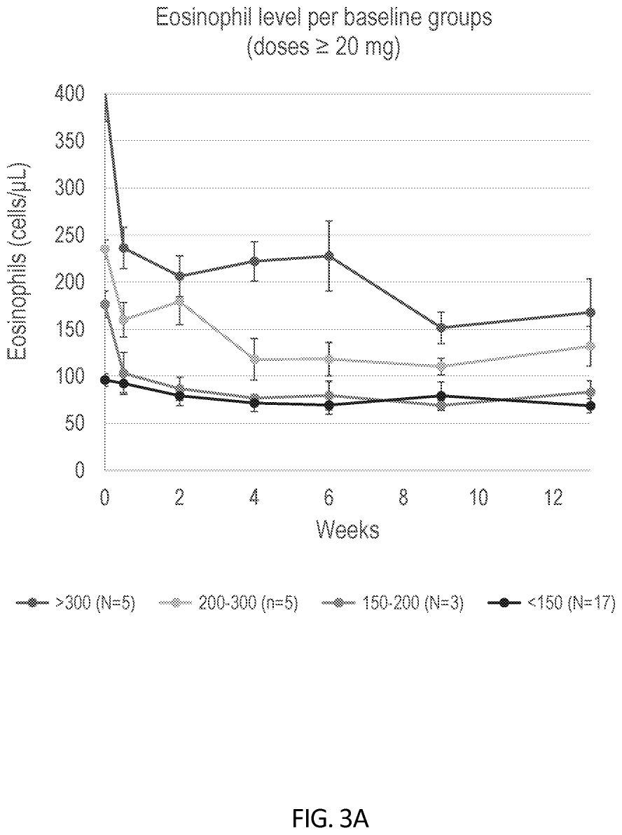

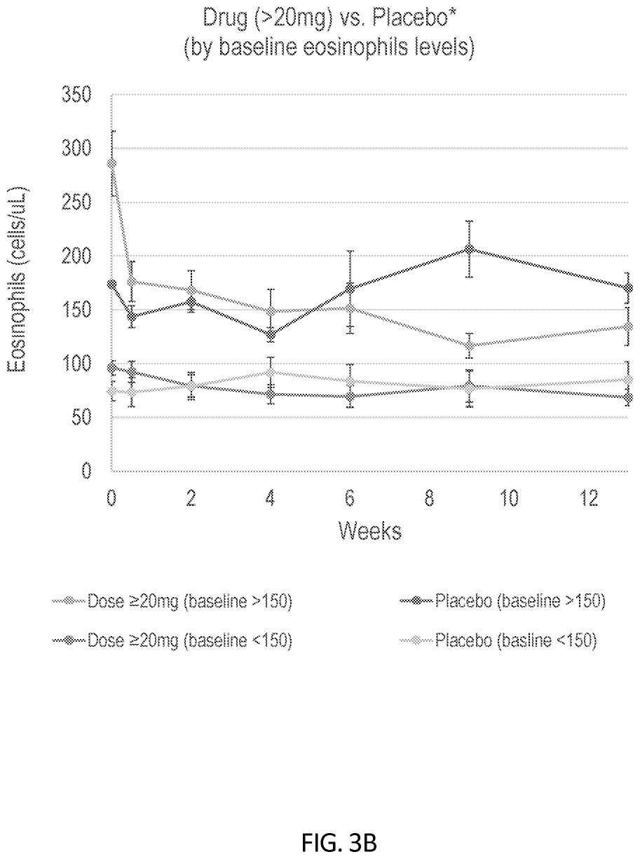

Leukocyte differentials were measured from peripheral blood. Eosinophil concentrations in healthy volunteers treated with greater than or equal to 20 mg (0.3 mg/kg) of TRAB-1 were monitored as a function of time (FIG. 3A). Groups were selected based on eosinophil concentrations at day 0: <150 cells/μL (n=17); 150-200 cells/μL (n=3); 200-300 cells/μL (n=5); and >300 cell/μL (n=5).

Eosinophil concentrations in healthy volunteers treated with greater than or equal to 20 mg (0.3 mg/kg) of TRAB-1 were monitored as a function of time and compared to the placebo group (FIG. 3B). Groups were selected based on eosinophil concentrations at day 0: <150 cells/μL (treatment: n=17; placebo: n=9); and >150 cells/μL (treatment: n=13; placebo: n=3). Data from one placebo subject, with high eosinophil baseline levels (646 cells/mL) was removed for being out of range. This data demonstrates that reduction of eosinophils was evident after a single dose of TRAB-1 in healthy volunteers.

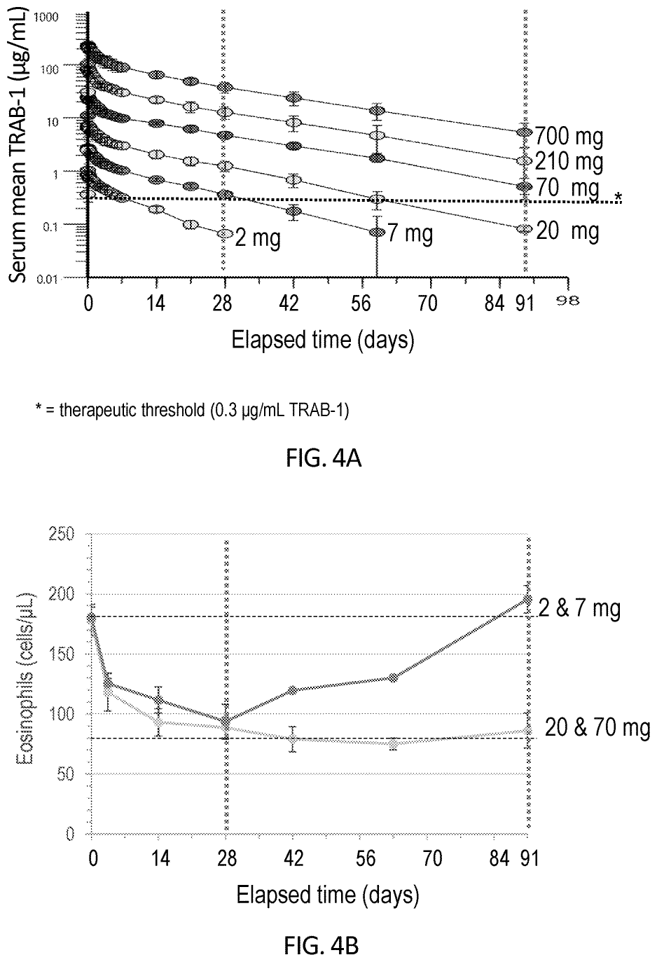

Pharmacokinetics curves depicting serum mean TRAB-1 were plotted as a function of time in healthy volunteers treated with 2 mg, 7 mg, 20 mg, 70 mg, 210 mg, or 700 mg of TRAB-1 (FIG. 4A). Subjects with the same baseline eosinophil levels (147-215 cells/μL) were compared across two dose groups (2 mg and 7 mg of TRAB-1, or 20 mg and 70 mg of TRAB-1), and the pharmacodynamics curves depicting eosinophils were plotted as a function of time (FIG. 4B). Low-doses were found to lose efficacy when serum concentrations of TRAB-1 dropped below the therapeutic threshold. Higher doses retained efficacy and TRAB-1 concentrations remained above the therapeutic threshold. These data depict that a pharmacokinetics/pharmacodynamics correlation in healthy volunteers treated with single ascending dose of TRAB-1 is indicated.

### Example 3: Multiple Ascending Dose Studies in Asthmatic Subjects

Multiple ascending dose (MAD) studies were performed in asthmatic subjects treated with TRAB-1 or placebo. The studies were randomized and double-blind. Observation continued for either 4, 8, 12, or 24 weeks (as indicated herein) following the last dose. Safety, tolerability, immunogenicity, pharmacokinetics, and pharmacodynamics were assessed. The pharmacodynamics assessed included target engagement, blood eosinophils, and fractional exhaled Nitric Oxide (Fractional exhaled Nitric Oxide (FeNO)) levels. A pharmacokinetics-pharmacodynamics profile, including the long tail of the pharmacodynamics response, was assessed. Immunogenicity was used to confirm explore pharmacodynamics and anti-drug antibodies. Exploratory studies were also performed to assess mechanism of action. The exploratory studies were also performed to assess affected pathways (e.g., Th2 and Th1 response). For example, serum concentrations of cytokines downstream to TSLP-R were assessed in exploratory studies.

The following dosing schedules were studied: single dose (SD), every four weeks (Q4W), every 8 weeks (Q8W), every 12 weeks (Q12W), every 16 weeks (Q16W), and every 24 weeks (Q24W). The following doses were selected for study: 25 mg, 100 mg, 200 mg, and 300 mg. The pharmaceutical dosage range per weight selected for study was 0.03-10 mg/kg per pharmaceutical dosage. The routes of administration selected for study was subcutaneous injection (SC). The dosing schedule for each participant group is indicated in the following examples.

Abbreviations used in the examples are as follow: SD=single dose; Q4W=every 4 weeks; Q8W=every 8 weeks; Q12W=every 12 weeks; Q16W=every 16 weeks; Q24W=every 24 weeks; IV=intravenous; SC=subcutaneous

The subjects were observed for treatment emergent aversive events and emergent adverse events. Mean serum concentration of TRAB-1 was monitored over time and was analyzed for dose-proportional pharmacokinetics. Bioavailability was measured. Antidrug-antibodies (ADAs) and their impact on pharmacokinetics was measured. Leukocyte differentials were measured from peripheral blood. Eosinophil concentrations in asthmatic subjects of TRAB-1 were monitored as a function of time and compared to the placebo group.

Pharmacokinetics and pharmacodynamics curves depicting serum mean TRAB-1 were monitored and plotted as a function of time in asthmatic subjects. Efficacy at low-doses and high doses were measured. The data was analyzed for a pharmacokinetics/pharmacodynamics correlation.

Receptor saturation is also measured.

### Results

As discussed above, a randomized, double-blind, placebo-controlled multiple ascending-dose study was conducted to evaluate the safety, tolerability, pharmacokinetics (PK), and pharmacodynamics (PD) of TRAB-1 patients with asthma. The cohort received either a placebo for a total of 3 injections, or TRAB-1 by subcutaneous (SC) injection. Of the subjects administered TRAB-1, Group 1 was administered 100 mg TRAB-1 every 4 weeks for a total of 2 or 3 administrations, Group 2 was administered 200 mg TRAB-1 every 4 weeks for a total of 1, 2, or 3 administrations, a Group 3 was administered 300 mg TRAB-1 every 12 weeks for a total of 1 or 2 administrations, and a Group 4 was administered 25 mg TRAB-1 for a total of 1 administration. Groups were observed for up to 24 weeks. Demographics data for each cohort is available in Table 7.

TABLE 7
Subject Demographics

| | Group 1: | Group 2: | Group 3: | | |
|---|---|---|---|---|---|
| TRAB-1 dosage | 100 mg | 200 mg | 300 mg | Group 4: | |
| and schedule | Q4W | Q4W | Q12W | 25 mg | Placebo |
| n | 6 | 6 | 6 | 5 | 8 |
| Mean age | 35.7 | 37.7 | 32.7 | 49.4 | 37.0 |
| Gender N (%) | 1M:5F | 2M:4F | 4M:2F | 3M:2F | 5M:3F |
| Male | 1 (17%) | 2 (33%) | 4 (67%) | 3 (60%) | 5 (63%) |
| Female | 5 (83%) | 4 (67%) | 2 (33%) | 2 (40%) | 3 (38%) |
| Race N (%) | | | | | |
| Asian | 2 (33%) | — | 3 (30%) | — | — |
| African | — | 1 (17%) | — | — | 1 (13%) |
| American/Black | | | | | |
| White | 4 (67%) | 4 (67%) | 2 (33%) | 5 (100%) | 7(88%) |
| Other or Mixed | — | 1 (17%) | 1 (17%) | — | — |
| Mean BMI | 26.6 | 25.2 | 25.2 | 26.8 | 25.6 |

Several subjects had mild, short-lived, and self-limited injection site reactions (ISR) TEAEs were observed in the groups administered 100 mg Q4W (83%; n=5/6), 200 mg Q4W (50%; n=3/6), and 300 mg Q12W (33%; n=2/6) of TRAB-1. All treatment emergent adverse events (TEAEs) that were observed are described in Table 8. All TEAEs were assigned a relatedness rating based on the likelihood of the TEAE being related to the treatment. All TEAEs were determined to be either possibly related to treatment (PR) or not related to treatment (NR). The TEAEs that were determined to be possibly related to treatment included respiratory infection, rhinitis, back stiffness, first degree heart block, and headaches. TEAEs that were determined to be not related to treatment included coryza (1 mild; 2 moderate) and blocked nose (2 mild). For Group 1, a total of 15 TEAEs (60% mild; 40% moderate) were observed, of which 2 mild TEAEs were determined to possibly be related to treatment, and 13 TEAEs were determined to be not related to treatment. For Group 2, a total of 8 TEAEs (63% mild; 38% moderate) were observed, of which 2 mild TEAEs were possibly be related to treatment, and 6 TEAEs were determined to be not related to treatment. For Group 3, a total of 3 TEAEs (100% mild) were observed, of which 2 were possibly be related to treatment, and 1 was determined to be not related to treatment. No TEAEs were observed for Group 4. For the placebo group, a total of 7 TEAEs (29% mild; 71% moderate) were observed, of which 1 mild TEAE was determined to possibly be related to treatment. No serious aversive events (SAEs) and no withdrawal symptoms were observed in any group. No treatments were not stopped for any individuals in any groups due to TEAEs. The most common TEAEs observed were headaches (HA) (see Table 9). For Group 1, 3 individuals reported a total of 4 headaches, of which 1 (250) was mild and possibly related to treatment, and 3 (75%) were moderate and determined to be not related to treatment. A single subject from the Group 3 experienced one mild headache that was determined to be possibly related to treatment.

TABLE 8
Treatment Emergent Adverse Events

| TRAB-1 | Group 1: 100 mg | | Group 2: 200 mg | | Group 3: 300 mg | | Group 4: | | |
|---|---|---|---|---|---|---|---|---|---|
| dosage | Q4 W | | Q4 W | | Q12 W | | 25 mg | Placebo | |
| and | N | | N | | N | | N | N | |
| schedule | (%) | Related | (%) | Related | (%) | Related | (%) | (%) | Related |
| # of | 5 | — | 3 | — | 2 | — | 0 | — | 5 |
| subjects | (83%) | | (50%) | | (33%) | | (0%) | | (83%) |
| with | | | | | | | | | |
| TEAEs | | | | | | | | | |
| Total | 15 | 2 PR | 8 | 2 PR | 3 | 1NR | 0 | 1 PR | 15 |
| number of | | 13 NR | | 6 NR | | 2 PR | | 4 NR | |
| TEAEs | | | | | | | | 2^{†} | |
| Mild (% | 9 | 7 NR | 5 | 2 PR | 3 | 1NR | 0 | 1^{†} | 9 |
| of TEAEs) | (60%) | | (63%) | 3 NR | (100%) | 2 PR | (0%) | | (60%) |
| Moderate | 6 | All NR | 3 | All NR | 0 | — | 0 | 3 NR | 6 |
| (% of | (40%) | | (38%) | | (0%) | | (0%) | 2^{†} | (40%) |
| TEAEs) | | | | | | | | | |
| Severe (% | 0 | — | 0 | — | 0 | — | 0 | — | 0 |
| of TEAEs) | (0%) | | (0%) | | (0%) | | (0%) | | (0%) |
| Withdrawal/ | 0 (0%) | | 0 (0%) | | 0 (0%) | | 0 | 0 (0%) | |
| IMP | | | | | | | (0%) | | |
| stopped | | | | | | | | | |

* Q4 W = every 4 weeks;
Q12 W = every 12 weeks;
PR = possibly related;
NR = not related;
^{†}= Determination pending

TABLE 9
Distribution of TEAEs reported to be headaches

| | | | Group 2: | Group 3: 300 mg | | | | |
|---|---|---|---|---|---|---|---|---|
| | Group 1: 100 mg | | 200 mg | Q12 W | | | Group 4: | |
| TRAB-1 dosage | Q4 W | | Q4 W | | | | 25 mg | Placebo |
| and schedule | N (%) | Relatedness | N (%) | N (%) | | Relatedness | N (%) | N (%) |
| # subjects with HA | 3 | — | 0 | 1 | | — | 0 | 0 |
| # HAs reported | 4 | 3 NR, 1 PR | 0 (0%) | 1 | | 1 PR | 0 (0%) | 0 (0%) |
| # Mild (% of HAs) | 1 (25%) | 1 PR | 0 (0%) | 1 | (100%) | 1 PR | 0 (0%) | 0 (0%) |
| # Moderate (% of | 3 (75%) | All NR | 0 (0%) | 0 | (0%) | — | 0 (0%) | 0 (0%) |

HAs)

* Q4 W = every 4 weeks;
Q12 W = every 12 weeks;
HA = headache;
TEAE = treatment emergent adverse event

Baseline values for each group for FEV1% predicted, mean FeNo, and mean Eosinophils were measured (Table 10).

TABLE 10
Baseline Characteristics

| TRAB-1 dosage | Group 1: 100 | | Group 2: 200 | | Group 3: 300 | | Group 4: 25 | | | |
|---|---|---|---|---|---|---|---|---|---|---|
| and schedule | mg Q4W | | mg Q4W | | mg Q12W | | mg | | Placebo | |
| FEV1% predicted | 89.1 | (6.7)% | 91.2 | (5.6)% | 98.1 | (7.6)% | 84.6 | (5.6)% | 89.5 | (5.4)% |
| (mean +/− SE) | | | | | | | | | | |
| Mean FeNO | 54 | (8.5) ppb | 75 | (26.4) ppb | 62 | (22.5) ppb | 53 | (20.1) ppb | 33 | (8.1) ppb |
| (mean +/− SE) | | | | | | | | | | |
| Mean Eosinophils | 336 | (55.8) | 317 | (37.4) | 230 | (30.9) | 403 | (71.5) | 241 | (26.4) |
| (mean +/− SE) | | | | | | | | | | |

* Q4W = every 4 weeks;
Q12W = every 12 weeks;
FEV1% = forced expiratory volume in the first second as a percentage of the predicted baseline value

No ADA positive samples were detected in samples tested from subjects receiving multiple doses of 100 mg or 200 mg TRAB-1.

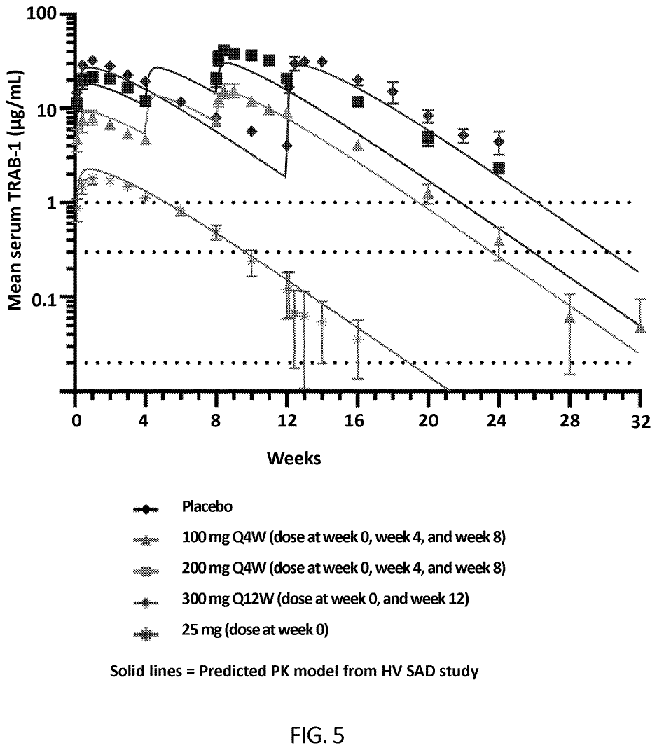

Mean serum concentrations of TRAB-1 were monitored and plotted as a function of time (FIG. 5). Mean serum concentrations were obtained for Group 1 (100 mg TRAB-1) in subjects administered 3 administrations of TRAB-1. For Group 2 and Group 3, mean serum concentrations are shown for the subjects after a single administration of TRAB-1. The observed serum concentrations observed (FIG. 5) matched the predicted serum concentrations from the model as shown in FIG. 2B.

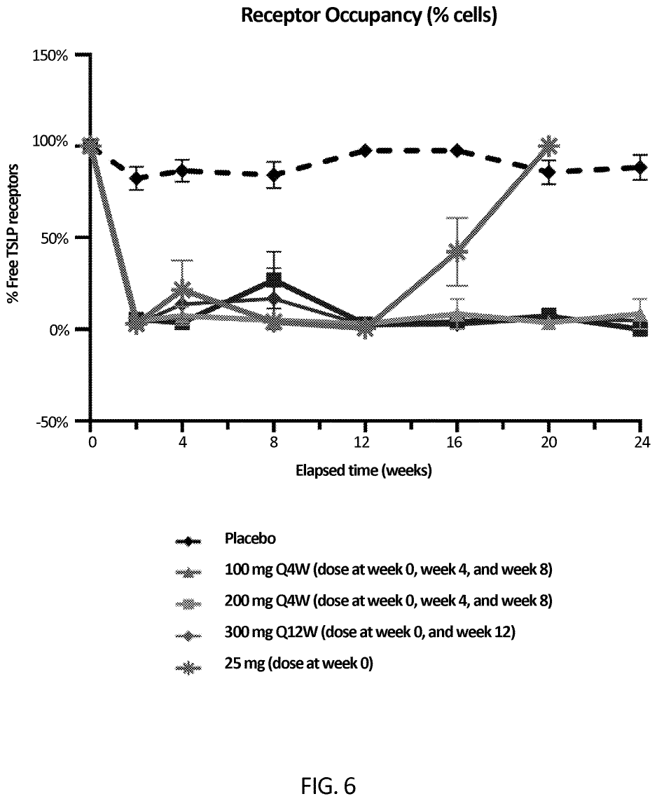

TSLP-R occupancy was measured as a function of time (FIG. 6) in asthmatic subjects administered placebo, or TRAB-1. For the subjects administered TRAB-1: Group 1 was administered 100 mg TRAB-1 every 4 weeks for a total of 2 or 3 administrations and receptor occupancy was monitored over 24 weeks; a Group 2 was administered 200 mg TRAB-1 for a total of 1, 2, or 3 administrations and receptor occupancy was monitored over 24 weeks; Group 3 was administered 300 mg TRAB-1 and receptor occupancy was monitored over 24 weeks. Group 4 was administered 25 mg TRAB-1 and receptor occupancy was monitored over 24 weeks. Initial receptor occupancy was approximately 100% available receptors for all subjects prior to any injections. Receptor occupancy was observed for up to 24 weeks for Group 1, Group 2, and Group 3, and up to 20 weeks for Group 4 and the placebo group. After one dose, receptor occupancy decreased to approximately 0% free at 2 weeks for Group 1, Group 2, and Group 3, but remained elevated for the placebo group. Receptor occupancy remained approximately 0% free up to 24 weeks for Group 1, Group 2, and Group 3, and up to 12 weeks for Group 4. For the placebo group, receptor occupancy remained elevated (approximately 100% free receptors) through 24 weeks.

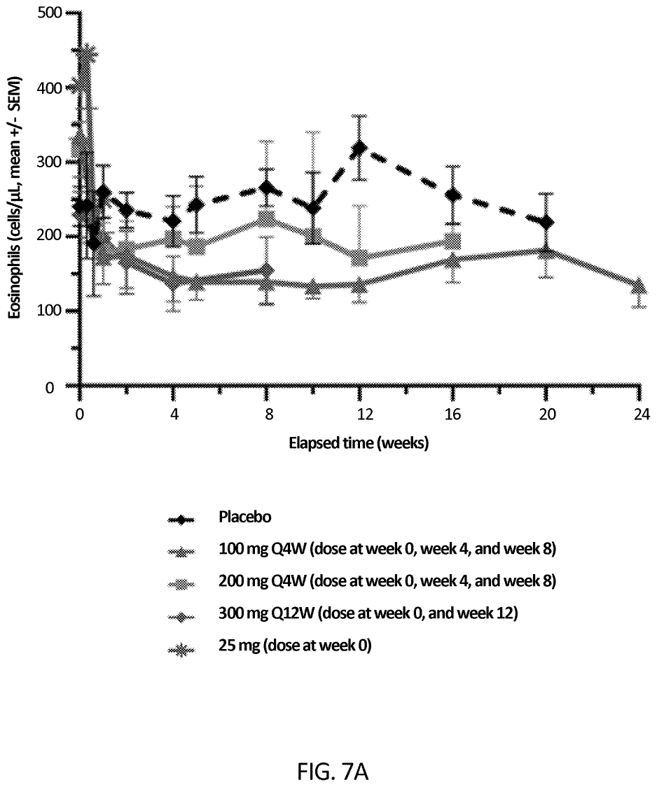

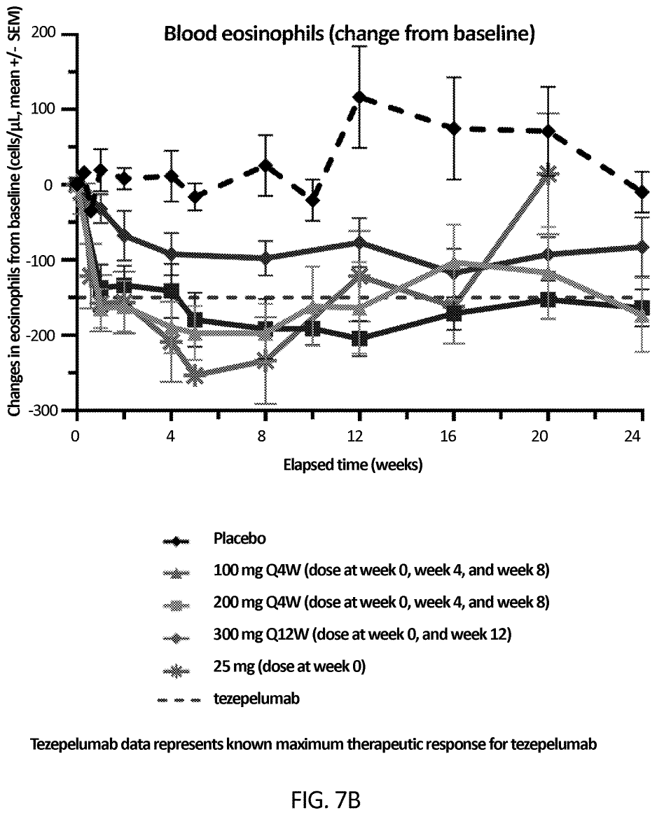

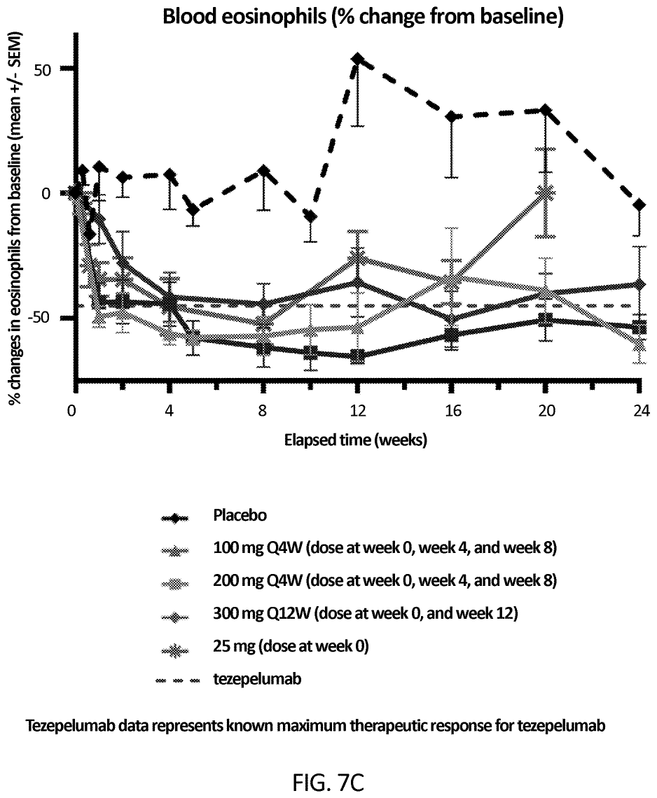

Eosinophil concentrations were monitored as a function of time in asthmatic subjects treated with TRAB-1 and compared to the placebo group (FIG. 7A). Group 1 was administered 100 mg TRAB-1 every 4 weeks for a total of 2 or 3 administrations and eosinophil concentration was monitored over 24 weeks; Group 2 was administered 200 mg TRAB-1 for a total of 1, 2, or 3 administrations and eosinophil concentration was monitored over 24 weeks; Group 3 was administered 300 mg TRAB-1 and eosinophil concentration was monitored over 24 weeks; and Group 4 was administered 25 mg TRAB-1 and eosinophil concentration was monitored over 20 weeks. Eosinophil concentrations were decreased at 1 week after 1 administration of Group 1, Group 2, Group 3, and Group 4. This data demonstrates that reduction of eosinophils was evident after in in asthmatic subjects administered 25 mg, 100 mg, 200 mg, or 300 mg of TRAB-1 after a single administration. At 12 weeks, a decrease in Eosinophils of approximately 164 cells/μL, 204 cells/μL, and 77 cells/μL were observed for Group 1, Group 2, and Group 3, respectively. These decreases were equivalent to an approximately a 54% decrease, 65% decrease, and 36% decrease compared to baseline (FIG. 7C), respectively. In comparison, approximately 150 cells/μL in eosinophils were observed in subjected administered 210 mg Tezepelumab (Teze Phase 3 study) after 12 weeks (Ly et al., J Clin Pharm 2021. July; 61(7):901-912). This data demonstrates that reduction of eosinophils was evident in asthmatic subjects that received 100 mg or 200 mg of TRAB-1 every 4 weeks for a total of 1, 2, or 3 dosages.

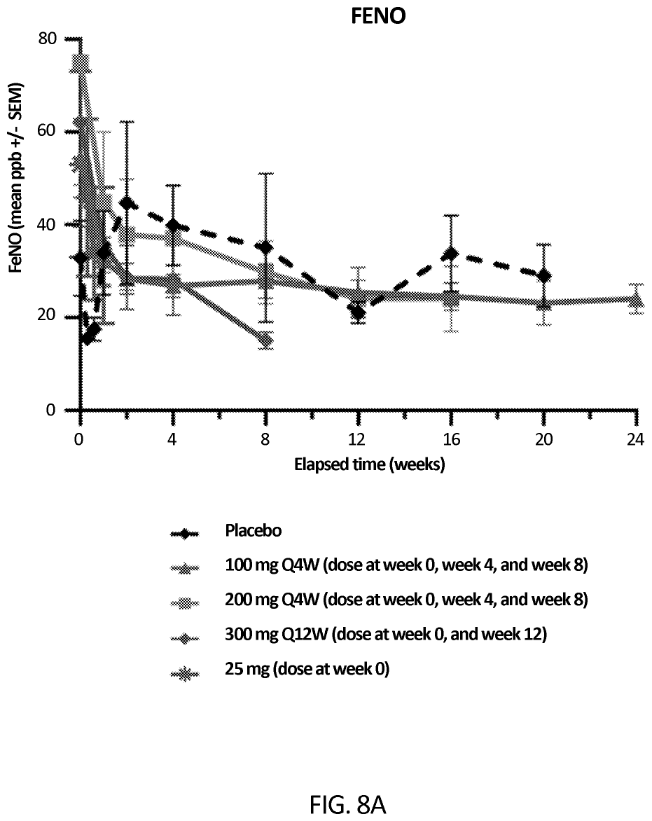

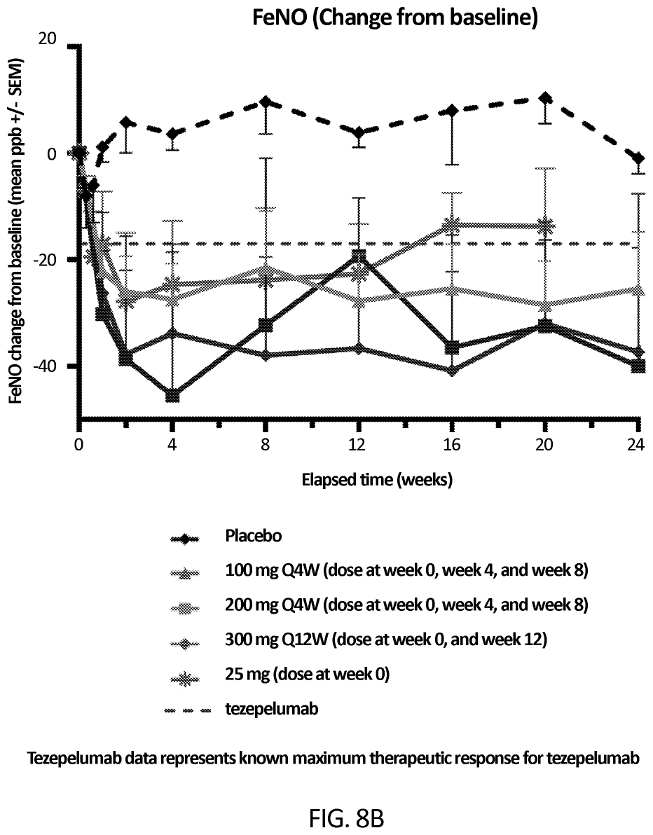

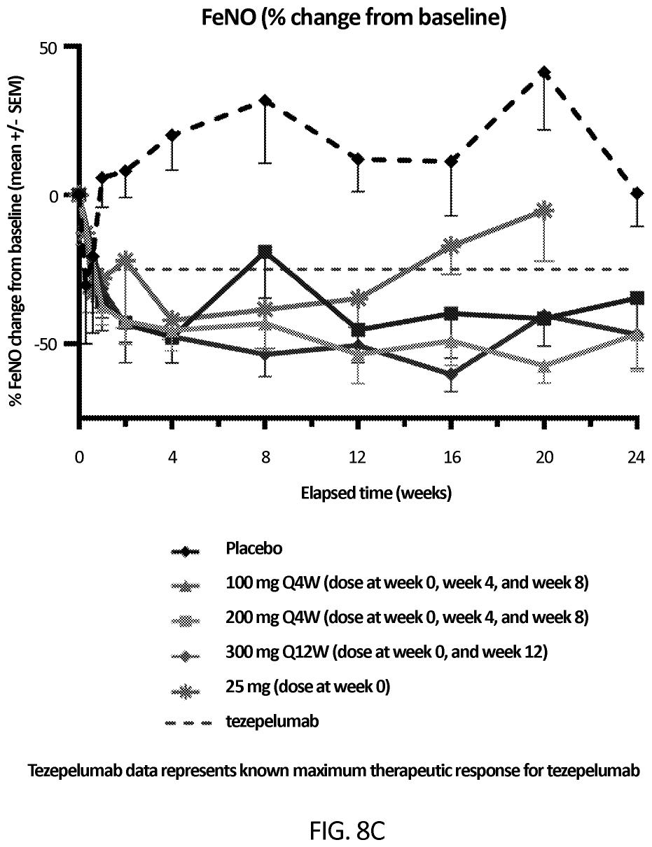

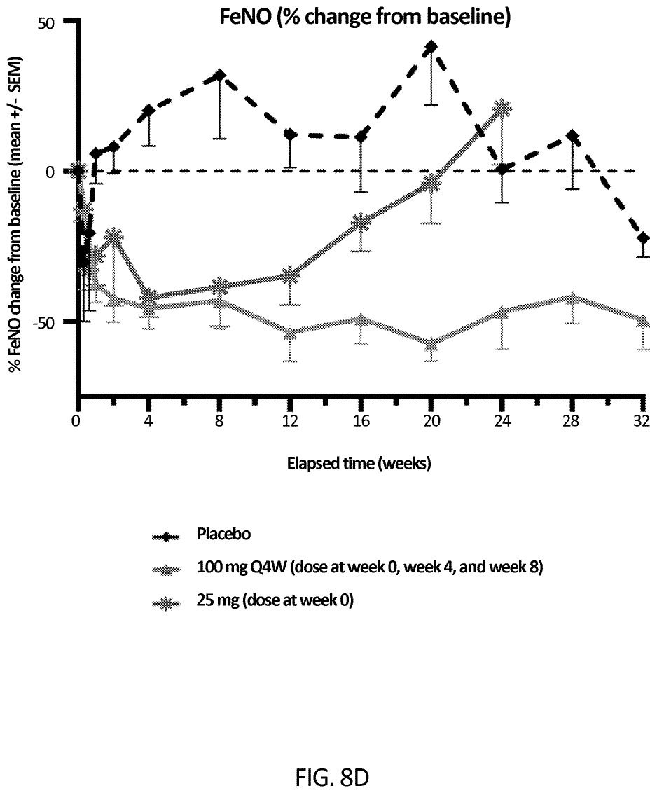

Fractional exhaled Nitric Oxide (FeNO) was monitored as a function of time (FIG. 8A) in asthmatic subjects administered placebo, or TRAB-1. Group 1 was administered 100 mg TRAB-1 every 4 weeks for a total of 3 administrations and FeNO was monitored over 32 weeks; a Group 2 was administered 200 mg TRAB-1 for a total of 1, 2, or 3 administrations and FeNO was monitored over 24 weeks; Group 3 was administered 300 mg TRAB-1 and FeNo was monitored over 2 4 weeks; and Group 4 was administered 25 mg TRAB-1 and FeNO was monitored over 24 weeks. FeNO were decreased at 4 weeks after 1 administration of Group 1, Group 2, Group 3, and Group 4. At 12 weeks, a decrease in FeNO of approximately 28 ppb, 19 ppb, and 37 ppb were observed in subjects in Group 1, Group 2, and Group 3, respectively (FIB. 8B). These decreases in FeNO were equivalent to an approximately a 54% decrease, 45% decrease, and 51% decrease compared to baseline (FIG. 8C), respectively. This data demonstrates that reduction of FeNO was evident after in the TRAB-1 in asthmatic subjects that received 100 mg of TRAB-1 every 4 weeks for a total of 3 dosages, up to 32 weeks after the administration of the first dosage and up to 24 weeks after the administration of the third dosage.

All available data on the effect of TRAB-1 on eosinophil levels and FeNO from the MAD study was used to generate a PK/PD model between TRAB-1 dose, TRAB-1 exposure (PK), and PD (FeNO or EOS) profiles in time. The maximum magnitude for response (Eₘₐₓ) was calculated to be a 42.6% reduction from baseline in eosinophil levels, and the concentration of TRAB-1 needed for a 50% response (EC₅₀) on eosinophil levels was calculated to be 0.000514 g/mL TRAB-1.

The Eₘₐₓ for FeNO response was calculated to be a 43.4% reduction from baseline in FeNO, and the concentration of TRAB-1 needed for a 50% response (EC₅₀) in FeNO was calculated to be 0.0514 g/mL TRAB-1. In comparison, an approximately 27.8% decrease in FeNO with an EC₅₀ of 2.5 g/mL was observed in subjected administered 210 mg Tezepelumab (Teze Phase 3 study) after 12 weeks (Ly et al., J Clin Pharm 2021. July; 61(7):901-912).

No subjects with clear loss of pharmacokinetics, FeNO, or eosinophil effect were observed.

### Example 4: Pharmacokinetic/Pharmacodynamic Modeling of TRAB-1 on FeNO Based on MAD Data

The relationship between the dosage of TRAB-1, the serum concentrations of TRAB-1 observed in patients immediately before the next dose was administered (Ct_{roug}h), and the effect on FeNO was further evaluated over time in a PK/PD model. The threshold concentration of TRAB-1 needed for a 50% response (EC₅₀), 80% response (EC₅₀), and 90% response (EC₉₀) on FeNO were calculated to be 0.1386 μg/ml, 0.062 μg/ml, and 0.015 μg/ml based on all available data in the MAD study. The population of patients demonstrating a response above the threshold concentration of TRAB-1 needed for a 50% response (EC₅₀), 80% response (EC₅₀), and 90% response (EC₉₀) on FeNO was compared for the following dosing regimens: 20 mg, 40 mg, 75 mg, 100 mg, 150 mg, 200 mg, and 300 mg TRAB-1 given every 12 weeks (Q12W); 400 mg TRAB-1 given every 24 weeks (Q24W); and 100 mg TRAB-1 given every 4 weeks (Q4W).

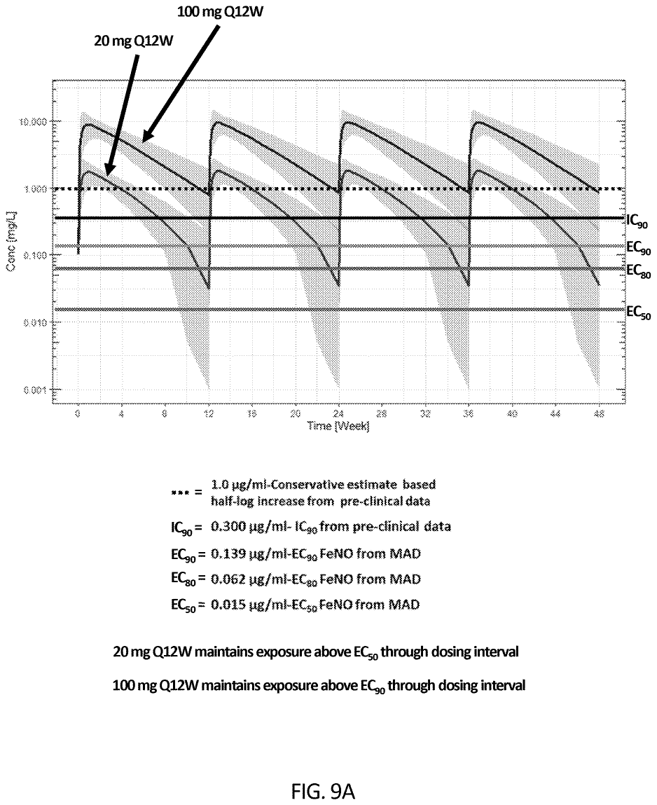

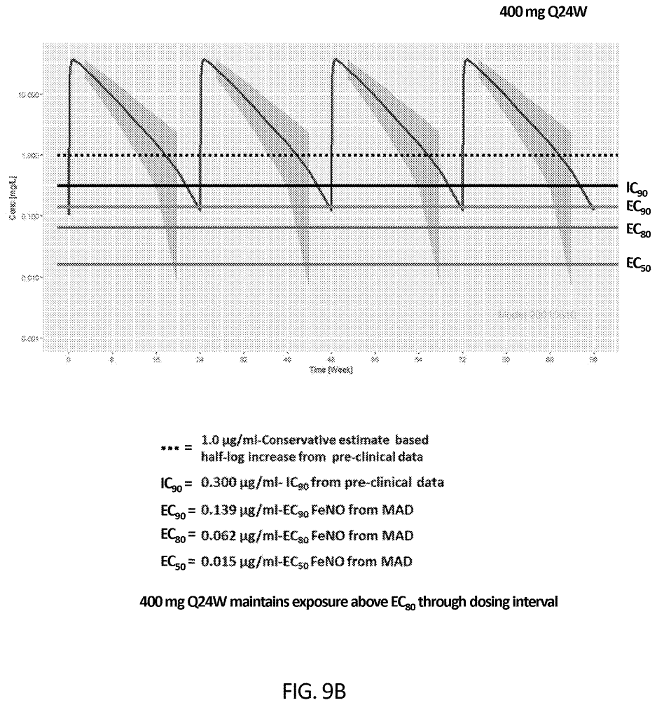

The 20 mg TRAB-1 Q12W dosing regimen was demonstrated to provide a trough exposure of greater than the EC₅₀ for FeNO in more than 60% of the population; of greater than the EC₈₀ for FeNO in 38.6% of the population; and of greater than the EC₉₀ for FeNO in 17.7% of the population (FIG. 9A). This data demonstrates that a dosing regimen of 20 mg TRAB-1 given every 12 weeks is a viable low dose regimen.

The 75 mg TRAB-1 Q12W and 100 mg TRAB-1 Q12W dosing regimens were demonstrated to provide trough exposures of greater than the EC₉₀ for FeNO in more 94.6% and 97.9% of the population, respectively. In comparison, less than 5% of patients treated with 70 mg tezepelumab every 4 weeks achieved a trough exposure greater than the EC₉₀ for FeNO (Ly et al., J Clin Pharm 2021. July; 61(7):901-912). Dosing regimens of 75 mg or more of TRAB-1 given every 12 weeks, and 100 mg of TRAB-1 given every 4 weeks, were demonstrated to produce near the maximum effect on FeNO in the majority of patients (Table 11A and Table 11B).

TABLE 11A
Proportion of patients above EC₅₀, EC₈₀, and EC₉₀ thresholds for
FeNO response at varying dosage regimens for TRAB-1 antibody.

| Threshold | Proportion above threshold at trough for given regimen | | | | |
|---|---|---|---|---|---|
| for FeNO | 20 mg | 400 mg | 40 mg | 75 mg | 100 mg |
| response | Q12W | Q24W | Q12W | Q12W | Q12W |
| EC50 | 60.4% | 68.2% | 94.9% | 99.6% | 99.8% |
| (0.0154 | | | | | |
| μg/mL) | | | | | |
| EC80 | 38.6% | 59.2% | 86.8% | 98.1% | 99.8% |
| (0.0616 | | | | | |
| μg/mL) | | | | | |
| EC90 | 17.7% | 48.0% | 73% | 94.6% | 97.9% |
| (0.1386 | | | | | |
| μg/mL) | | | | | |

TABLE 11B
Proportion of patients above EC₅₀, EC₈₀, and EC₉₀ thresholds for
FeNO response at varying dosage regimens for TRAB-1 antibody.

| Threshold | Proportion above threshold at trough for given regimen | | | |
|---|---|---|---|---|
| for FeNO | 150 mg | 200 mg | 300 mg | 100 mg |
| response | Q12W | Q12W | Q12W | Q4W |
| EC50 | 99.8% | 100% | 100% | 100% |
| (0.0154 | | | | |
| μg/mL) | | | | |
| EC80 | 99.8% | 100% | 100% | 100% |
| (0.0616 | | | | |
| μg/mL) | | | | |
| EC90 | 99.6% | 99.7% | 99.8% | 100% |
| (0.1386 | | | | |
| μg/mL) | | | | |

In comparison, based on published data a dosing regimen of 280 mg tezepelumab given every 4 weeks (Q4W) is needed to produce near the maximum effect on FeNO in the majority of patients (Ly et al., J Clin Pharm 2021. July; 61(7):901-912).

The 400 mg TRAB-1 Q24W dosing regimen was demonstrated to provide a trough exposure of greater than the EC₅₀ for FeNO in 59.200 of the population; and of greater than the EC₉₀ for FeNO in more 4800 of the population (FIG. 9B).

This embodiments and data provided herein demonstrate that a dosing regimen of 400 mg TRAB-1 administered every 24 weeks to a subject can be used to achieve a consistent and durable therapeutic effect mediated by TRAB-1, such as for treating the indications and using the methods provided for herein. As provided for herein, although the composition may be administered every 24 weeks, other administration periods may be used with the same or different doses as provided for herein. For example, the PK/PD modeling based on the clinical data also demonstrates that dosing regimens of 100 mg of TRAB-1 given every 12 weeks or 200 of TRAB-1 given every 12 weeks can be therapeutically effective. In summary, it was demonstrated that TRAB-1 provided a greater effect on FeNO than what has been published regarding the effects of tezepelumab. This effect was achieved with lower dosages of TRAB-1 and less frequent dosing.

### Example 5: Clinical Trial in Subjects with Chronic Rhinosinusitis with Nasal Polyps

Single ascending dose (SAD) and multiple ascending dose (MAD) studies are performed in patients with Chronic Rhinosinusitis with Nasal Polyps treated with TRAB-1 or placebo. A third group is also considered, wherein the third group is treated with an anti-TSLP antibody, e.g., tezepelumab.

The studies are randomized and double-blind. Observation will continue for either 4, 8, 12, 24, 48, or 52 weeks following the last dose. Safety, tolerability, immunogenicity, pharmacokinetics, and pharmacodynamics are assessed. The pharmacodynamics assessed will include target engagement, blood eosinophils, and Fractional exhaled Nitric Oxide (FeNO). A pharmacokinetics-pharmacodynamics profile, including the long tail of the pharmacodynamics response, is assessed. Immunogenicity will be used to confirm whether pharmacodynamics and anti-drug antibodies support further development. Exploratory studies are performed to assess mechanism of action. The exploratory studies are also performed to assess affected pathways (e.g., Th2 and Th1 response). For example, serum concentrations of cytokines downstream to TSLP-R are assessed in exploratory studies. Symptoms of Chronic Rhinosinusitis with Nasal Polyps are monitored.

The subjects are divided into groups, according to the severity of a nasal polyp-associated parameter according to as defined by nasal polyp-associated parameters chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes.

The following dosing schedules are studied: single dose (SD), every four weeks (Q4W), every 8 weeks (Q8W), every 12 weeks (Q12W), every 16 weeks (Q6W), and every 24 weeks (Q24W). The following doses are selected for study: 20 mg-30 mg, 50 mg, 100 mg, 200 mg, 250 mg, 300 mg, 400 mg, 500 mg, 600 mg, 700 mg, and 800 mg. The pharmaceutical dosage range per weight selected for study is 0.03-10 mg/kg per pharmaceutical dosage. The routes of administration selected for study are intravenous (IV) and subcutaneous (SC) routes. The dosing schedule selected for study is summarized in Table 12.

TABLE 12
Dosing schedule.

| | | Route of |
|---|---|---|
| Schedule | Pharmaceutical dosage | administration |
| SD | 20 mg-30 mg, 50 mg, 100 mg, 150 mg, 200 mg, 250 mg, 300 | IV, SC |
| | mg, 350 mg, 400 mg, 500 mg, 600 mg, 700 mg, 800 mg | |
| Q4W | 20 mg-30 mg, 50 mg, 100 mg, 150 mg, 200 mg, 250 mg, 300 | IV, SC |
| | mg | |
| Q8W | 20 mg-30 mg, 50 mg, 100 mg, 150 mg, 200 mg, 250 mg, 300 | IV, SC |
| | mg | |
| Q12W | 20 mg-30 mg, 50 mg, 100 mg, 150 mg, 200 mg, 250 mg, 300 | IV, SC |
| | mg | |
| Q16W | 100 mg, 200 mg, 250 mg, 300 mg, 350 mg, 400 mg, 500 mg, | IV, SC |
| | 600 mg, 700 mg, 800 mg | |
| Q24W | 100 mg, 200 mg, 250 mg, 300 mg, 350 mg, 400 mg, 500 mg, | IV, SC |
| | 600 mg, 700 mg, 800 mg | |

Q16W = every 16 weeks;
Q24W = every 24 weeks;
IV = intravenous;
SC = subcutaneous

The TRAB-1 is administered on schedule, for up to 52 weeks.

The subjects are observed for treatment emergent aversive events and emergent adverse events. Mean serum concentration of TRAB-1 is monitored over time and is analyzed for dose-proportional pharmacokinetics. Bioavailability is measured. Antidrug-antibodies (ADAs) and their impact on pharmacokinetics is measured. Leukocyte differentials are measured from peripheral blood. Eosinophil concentrations in healthy volunteers treated with greater than or equal to 20 mg (0.3 mg/kg) of TRAB-1 are monitored as a function of time. Eosinophil concentrations in healthy volunteers treated with greater than or equal to 20 mg (0.3 mg/kg) of TRAB-1 are monitored as a function of time and compared to the placebo group.

Pharmacokinetics and pharmacodynamics curves depicting serum mean TRAB-1 are monitored and plotted as a function of time in healthy volunteers. Efficacy at low-doses and high doses are measured. The data is analyzed for a pharmacokinetics/pharmacodynamics correlation.

The symptoms monitored are measured according to assessments of nasal polyp-associated parameters chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or an assessment of patient reported outcomes.

For initial clinical trials, subjects selected for study are free of co-morbidities common in patients with Chronic Rhinosinusitis with Nasal Polyps. In additional studies, subjects selected for study have a co-morbidities common in patients with Chronic Rhinosinusitis with Nasal Polyps.

The clinical trials described herein may include a second therapeutic agent. The clinical trials described herein may include a second therapeutic agent indicated for the treatment of Chronic Rhinosinusitis with Nasal Polyps. The second therapeutic agent can be: levofloxacin, ciprofloxacin, moxifloxacin, sulfamethoxazole/trimethoprim, acetaminophen/caffeine, amoxicillin, ampicillin, oxacillin, amoxicillin/clavulante, ampicillin/sulbactam, chlorpheniramine/pseudoephedrine, dexchlorpheniramine/dextromethorphan/pseudoephedrine, diphenhydramine/phenylephrine, dextromethorphan/diphenhydramine/phenylephrine, dextromethorphan/phenylephrine/pyrilamine, cefuroxime, cefprozil, cefaclor, cefuroxime, cefprozil, cefaclor, cefdinir, cefixime, cefpodoxime, ceftazidime, clindamycin, prednisone, fluticasone, mometasone, beclomethasone, azithromycin, clarithromycin, omalizumab, dupilumab, mepolizumab, or any combination thereof.

In further studies, the secondary therapeutic may be a biologic. The biologic can be an antibody. The antibody can be an interleukin inhibitor, a TSLP inhibitor, or an IgG inhibitor. The interleukin inhibitor may be: dupi, CPB-201, CM-310, cendakimab, AK-120, eblasakimab, BC-005, QX-005N, 611, vaccines against IL-4/IL-13, dupi biosimilar, benralizumab, mepolizumab, reslizumab, or 610. The anti-TSLP inhibitor can be: tezepelumab, CSJ-117, or CM-326. The anti-IgE inhibitor can be: omalizumab, ligelizumab, FB-825, or UB-221. The second therapeutic may also be siglec.

In one treatment example, the subject is administered the pharmaceutical composition comprising an anti-TSLP-R antibody, or antigen-binding fragment thereof, by subcutaneous injection. In one treatment example, the subject is administered the pharmaceutical composition comprising an anti-TSLP-R antibody, or antigen-binding fragment thereof, by intravenous injection.

In one treatment example, the subject is administered the pharmaceutical composition at a single dose of about 1.0 mg/kg to about 15.0 mg/kg of the anti-TSLP-R antibody, or antigen-binding fragment thereof. In one treatment example, the subject is administered a single doses of about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg of the anti-TSLP-R antibody, or antigen-binding fragment thereof.

In one treatment example, the subject is administered multiple doses of the anti-TSLP-R antibody, or antigen-binding fragment thereof, wherein each dose has about 1.0 mg/kg to about 15.0 mg/kg of the anti-TSLP-R antibody, or antigen-binding fragment thereof. In one treatment example, the subject is administered a multiples doses of about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg of the anti-TSLP-R antibody, or antigen-binding fragment thereof, wherein each dose is administered every 1 week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, every 20 weeks, every 21 weeks, every 22 weeks, every 23 weeks, every 24 weeks, every 25 weeks, every 26 weeks, every 27 weeks, every 28 weeks, every 29 weeks, every 30 weeks, every 31 weeks, every 32 weeks, every 33 weeks, every 34 weeks, every 35 weeks, every 36 weeks, every 37 weeks, every 38 weeks, every 39 weeks, every 40 weeks, every 41 weeks, every 42 weeks, every 43 weeks, every 44 weeks, every 45 weeks, every 46 weeks, every 47 weeks, every 48 weeks, every 49 weeks, every 50 weeks, every 51 weeks, or every 52 weeks.

In one treatment example, the subject is administered multiple doses of about 20 mg, 25 mg, 50 mg, 100 mg, 150 mg, 200 mg, 250 mg, 300 mg, or 400 mg of the anti-TSLP-R antibody, or antigen-binding fragment thereof, wherein each dose is administered every 4 weeks.

In one treatment example, the subject is administered multiple doses of the anti-TSLP-R antibody, or antigen-binding fragment thereof, every 4 weeks for a total of at least 4 weeks from the first dose, for a total of 2 dosages. In one treatment example, the subject is administered multiple doses of the anti-TSLP-R antibody, or antigen-binding fragment thereof, every 4 weeks for a total of at least 8 weeks from the first dose, for a total of 3 dosages. In one treatment example, the subject is administered multiple doses of the anti-TSLP-R antibody, or antigen-binding fragment thereof, every 4 weeks for a total of at least 12 weeks from the first dose, for a total of 4 dosages. In one treatment example, the subject is administered multiple doses of the anti-TSLP-R antibody, or antigen-binding fragment thereof, every 4 weeks for a total of at least 16 weeks from the first dose, for a total of 5 dosages. In one treatment example, the subject is administered multiple doses of the anti-TSLP-R antibody, or antigen-binding fragment thereof, every 4 weeks for at least 20 weeks. In one treatment example, the subject is administered multiple doses of the anti-TSLP-R antibody, or antigen-binding fragment thereof, every 4 weeks for at least 24 weeks. In one treatment example, the subject is administered multiple doses of the anti-TSLP-R antibody, or antigen-binding fragment thereof, every 4 weeks for a total of at least 28 weeks from the first dose, for a total of 6 dosages. In one treatment example, the subject is administered multiple doses of the anti-TSLP-R antibody, or antigen-binding fragment thereof, every 4 weeks for a total of at least 32 weeks from the first dose, for a total of 7 dosages. In one treatment example, the subject is administered multiple doses of the anti-TSLP-R antibody, or antigen-binding fragment thereof, every 4 weeks for a total of at least 36 weeks from the first dose, for a total of 8 dosages. In one treatment example, the subject is administered multiple doses of the anti-TSLP-R antibody, or antigen-binding fragment thereof, every 4 weeks for a total of at least 40 weeks from the first dose, for a total of 9 dosages. In one treatment example, the subject is administered multiple doses of the anti-TSLP-R antibody, or antigen-binding fragment thereof, every 4 weeks for a total of at least 44 weeks from the first dose, for a total of 10 dosages. In one treatment example, the subject is administered multiple doses of the anti-TSLP-R antibody, or antigen-binding fragment thereof, every 4 weeks for a total of at least 48 weeks from the first dose, for a total of 11 dosages. In one treatment example, the subject is administered multiple doses of the anti-TSLP-R antibody, or antigen-binding fragment thereof, every 4 weeks for a total of at least 52 weeks from the first dose, for a total of 12 dosages.

In one treatment example, the subject is administered multiple doses of about 20 mg, 25 mg, 50 mg, 100 mg, 150 mg, 200 mg, 250 mg, 300 mg, or 400 mg of the anti-TSLP-R antibody, or antigen-binding fragment thereof, wherein each dose is administered every 8 weeks.

In one treatment example, the subject is administered multiple doses of the anti-TSLP-R antibody, or antigen-binding fragment thereof, every 8 weeks for a total of at least 8 weeks from the first dose, for a total of 2 dosages. In one treatment example, the subject is administered multiple doses of the anti-TSLP-R antibody, or antigen-binding fragment thereof, every 8 weeks for a total of at least 16 weeks from the first dose, for a total of 3 dosages. In one treatment example, the subject is administered multiple doses of the anti-TSLP-R antibody, or antigen-binding fragment thereof, every 8 weeks for at least 24 weeks. In one treatment example, the subject is administered multiple doses of the anti-TSLP-R antibody, or antigen-binding fragment thereof, every 8 weeks for a total of at least 32 weeks from the first dose, for a total of 4 dosages. In one treatment example, the subject is administered multiple doses of the anti-TSLP-R antibody, or antigen-binding fragment thereof, every 8 weeks for a total of at least 40 weeks from the first dose, for a total of 5 dosages. In one treatment example, the subject is administered multiple doses of the anti-TSLP-R antibody, or antigen-binding fragment thereof, every 8 weeks for a total of at least 48 weeks from the first dose, for a total of 6 dosages.

In one treatment example, the subject is administered multiple doses of about 20 mg, 25 mg, 50 mg, 100 mg, 150 mg, 200 mg, 250 mg, 300 mg, or 400 mg of the anti-TSLP-R antibody, or antigen-binding fragment thereof, wherein each dose is administered every 12 weeks. In one treatment example, the subject is administered multiple doses of the anti-TSLP-R antibody, or antigen-binding fragment thereof, every 12 weeks for a total of at least 12 weeks from the first dose, for a total of 2 dosages. In one treatment example, the subject is administered multiple doses of the anti-TSLP-R antibody, or antigen-binding fragment thereof, every 12 weeks for at least 24 weeks. In one treatment example, the subject is administered multiple doses of the anti-TSLP-R antibody, or antigen-binding fragment thereof, every 12 weeks for a total of at least 36 weeks from the first dose, for a total of 3 dosages. In one treatment example, the subject is administered multiple doses of the anti-TSLP-R antibody, or antigen-binding fragment thereof, every 12 weeks for a total of at least 48 weeks from the first dose, for a total of 4 dosages.

In one treatment example, the subject is administered multiple doses of about 100 mg of the anti-TSLP-R antibody, or antigen-binding fragment thereof, wherein each dose is administered every 12 weeks. In one treatment example, the subject is administered multiple doses of the anti-TSLP-R antibody, or antigen-binding fragment thereof, every 12 weeks for a total of at least 12 weeks from the first dose, for a total of 2 dosages. In one treatment example, the subject is administered multiple doses of the anti-TSLP-R antibody, or antigen-binding fragment thereof, every 12 weeks for at least 24 weeks. In one treatment example, the subject is administered multiple doses of the anti-TSLP-R antibody, or antigen-binding fragment thereof, every 12 weeks for a total of at least 36 weeks from the first dose, for a total of 3 dosages. In one treatment example, the subject is administered multiple doses of the anti-TSLP-R antibody, or antigen-binding fragment thereof, every 12 weeks for a total of at least 48 weeks from the first dose, for a total of 4 dosages.

In one treatment example, the subject is administered multiple doses of about 100 mg, 200 mg, 250 mg, 300 mg, 350 mg, 400 mg, 500 mg, 600 mg, 700 mg, or 800 mg of the anti-TSLP-R antibody, or antigen-binding fragment thereof, wherein each dose is administered every 16 weeks. In one treatment example, the subject is administered multiple doses of the anti-TSLP-R antibody, or antigen-binding fragment thereof, every 16 weeks for a total of at least 16 weeks from the first dose, for a total of 2 dosages. In one treatment example, the subject is administered multiple doses of the anti-TSLP-R antibody, or antigen-binding fragment thereof, every 15 weeks for at least 32 weeks. In one treatment example, the subject is administered multiple doses of the anti-TSLP-R antibody, or antigen-binding fragment thereof, every 16 weeks for a total of at least 48 weeks from the first dose, for a total of 3 dosages.

In one treatment example, the subject is administered multiple doses of about 100 mg, 200 mg, 250 mg, 300 mg, 350 mg, 400 mg, 500 mg, 600 mg, 700 mg, or 800 mg of the anti-TSLP-R antibody, or antigen-binding fragment thereof, wherein each dose is administered every 24 weeks. In one treatment example, the subject is administered multiple doses of the anti-TSLP-R antibody, or antigen-binding fragment thereof, every 24 weeks for a total of at least 24 weeks from the first dose, for a total of 2 dosages. In one treatment example, the subject is administered multiple doses of the anti-TSLP-R antibody, or antigen-binding fragment thereof, every 24 weeks for a total of at least 48 weeks from the first dose, for a total of 3 dosages.

Patients with Chronic Rhinosinusitis with Nasal Polyps are predicted to have an improvement in one or more symptom of CRSwNP following administration of the pharmaceutical composition compared to the placebo group. The improvement of the one or more symptom of CRSwNP may occur after, or about, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or over time of treatment with the pharmaceutical composition comprising the TSLP-R antibody, or antigen-binding fragment thereof. The one or more symptom of CRSwNP may continue to improve after administration of the pharmaceutical composition comprising the TSLP-R antibody, or antigen-binding fragment thereof, has ceased for at least 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks. The subject may have a sustained improvement in one or more symptom of CRSwNP about 1 week, about 2 weeks, about 3 weeks, about weeks, 4 weeks, about weeks, 5 weeks, about weeks, 6 weeks, about weeks, 7 weeks, about weeks, 8 weeks, about weeks, 9 weeks, about weeks, 10 weeks, about weeks, 11 weeks, about weeks, 12 weeks, about weeks, 13 weeks, about weeks, 14 weeks, about weeks, 15 weeks, about weeks, 16 weeks, about weeks, 17 weeks, about weeks, 18 weeks, about weeks, 19 weeks, about weeks, 20 weeks, about weeks, 21 weeks, about weeks, 22 weeks, about weeks, 23 weeks, about weeks, 24 weeks, about weeks, 25 weeks, about weeks, 26 weeks, about weeks, 27 weeks, about weeks, 28 weeks, about weeks, 29 weeks, about weeks, 30 weeks, about weeks, 31 weeks, about weeks, 32 weeks, about weeks, 33 weeks, about weeks, 34 weeks, about weeks, 35 weeks, about weeks, 36 weeks, about weeks, 37 weeks, about weeks, 38 weeks, about weeks, 39 weeks, about weeks, 40 weeks, about weeks, 41 weeks, about weeks, 42 weeks, about weeks, 43 weeks, about weeks, 44 weeks, about weeks, 45 weeks, about weeks, 46 weeks, about weeks, 47 weeks, about weeks, 48 weeks, about weeks, 49 weeks, about weeks, 50 weeks, about weeks, about 51 weeks, or about 52 weeks after treatment has ended. The subject may not experience worsening of one or more symptom of CRSwNP after about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, or up to 52 weeks after treatment has ended.

The improvement may be an improvement in one or more symptom of CRSwNP chosen from loss of smell, nasal congestion, anterior nasal discharge, posterior nasal discharge, nasal peak inspiratory flow (NPIF) that occur in the day (AM), nasal peak inspiratory flow (NPIF) that occur in the night (PM), facial pain, headache, or any combination thereof.

The improvement may be an improvement in nasal polyp-associated parameters chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, and patient reported outcomes, or patient reported outcomes. The improvement may be an improvement in one or more patient reported outcomes according to one or more assessment chosen from the Short-Form-36 (SF-36) Questionnaire, the Eurogol-5D (EQ-5D), nasal polyp related resource use questionnaire, the patient qualitative self-assessment, or any combination thereof, compared to baseline. The improvement may be an improvement in a nasal polyp-associated biomarker, compared to baseline. The improvement may be an improvement in a nasal polyp-associated biomarker chosen from IgE, TARC, eotaxin-3 and ECP, compared to baseline. The improvement may be an improvement in the size of one or more nasal polyp as assessed by the nasal polyp score (NPS) compared to baseline. The improvement may be an improvement in the number of nasal polyps present compared to baseline. The improvement may be an improvement wherein the need for treatment with intranasal or oral corticosteroids is reduced in the subject. The improvement may be an improvement wherein the need for treatment with nasal surgery is reduced in the subject.

### Example 6: Phase 2 VIBRANT Trial Evaluating Verekitug (TRAB-1) in Participants with Chronic Rhinosinusitis with Nasal Polyps (CRSwNP)

CRSwNP is a chronic inflammatory disease of the upper airway, marked by inflammation in the nose and sinuses and the presence of nasal polyps. CRSwNP has four main symptoms: runny nose or postnasal drip, nasal congestion, facial pressure and/or pain and loss of smell and/or taste. Despite available treatments such as corticosteroids, surgery and, more recently, biologics, the quality-of-life studies and post-surgical recurrence rates clearly show that many people with CRSwNP have uncontrolled symptoms that are impacting their daily life and current treatments are not meeting their needs. It is estimated that CRSwNP affects up to 4% of the general population, of whom 40% have uncontrolled disease.

Nasal polyps are associated with significant disease burden and debilitating symptoms; it is estimated that over 40% of people with severe asthma also have CRSwNP and that up to 70% of people with CRSwNP also have asthma, demonstrating a strong association between the two conditions.

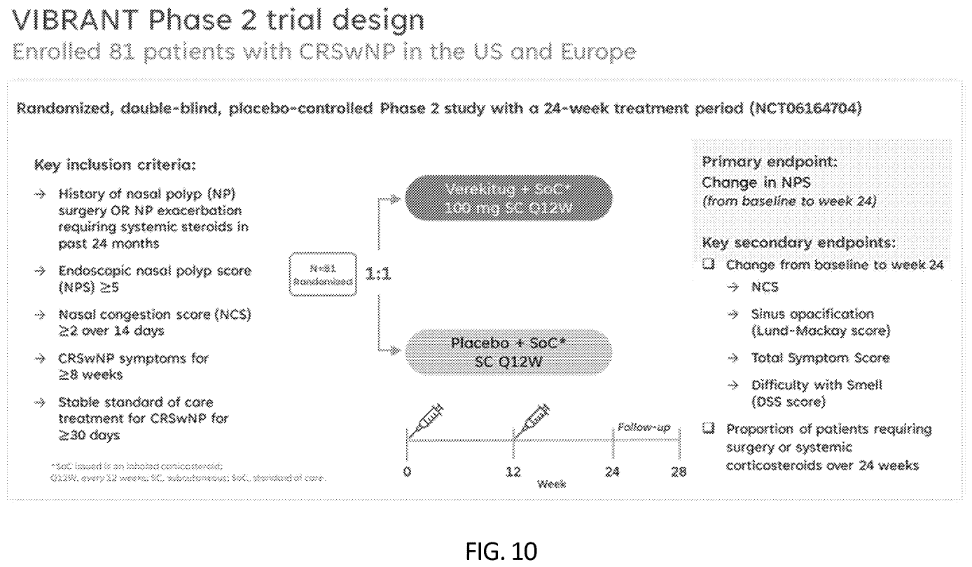

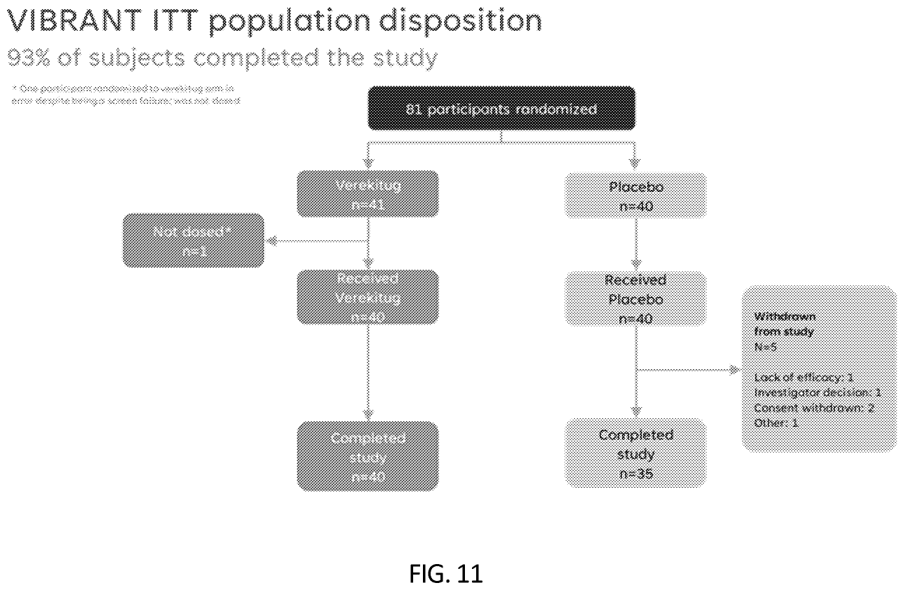

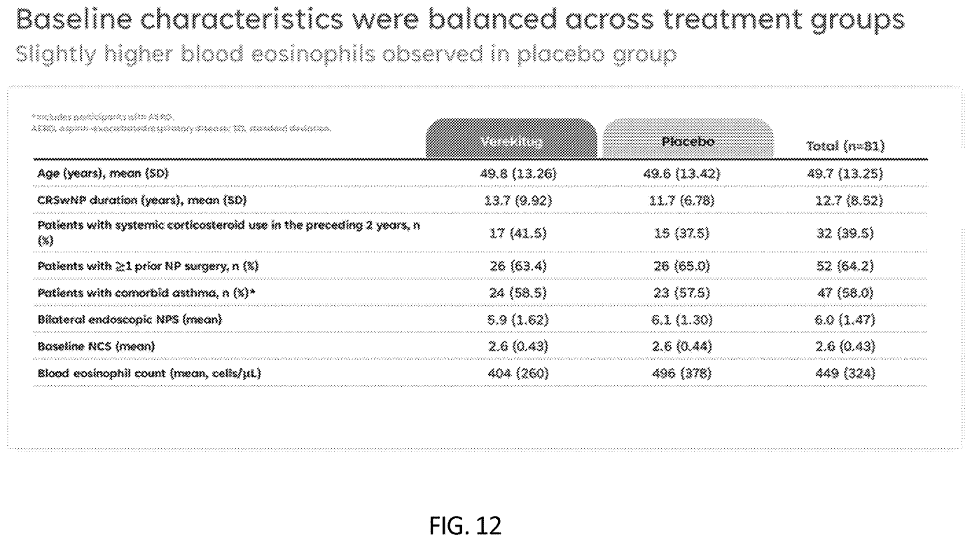

The Phase 2 VIBRANT trial was a global, randomized, placebo-controlled, parallel group clinical trial, which was designed to assess the efficacy and safety of verekitug in adults with CRSwNP who were receiving concurrent intranasal corticosteroid therapy (FIG. 10). Inclusion criteria included: history of nasal polyp (NP) surgery OR NP exacerbation requiring systemic steroids in past 24 months; endoscopic nasal polyp score (NPS)≥5; nasal congestion score (NCS)≥2 over 14 days; CRSwNP symptoms for ≥8 weeks; and stable standard of care treatment for CRSwNP for ≥30 days (FIG. 12). Participants received either 100 mg of verekitug or placebo subcutaneously every 12 weeks for 24 weeks (FIG. 11). The primary endpoint was change in endoscopic nasal polyp score (NPS) at Week 24, a primary endpoint that has been used in several registrational trials for other biologic treatments for CRSwNP. Secondary endpoints included: nasal congestion score (NCS), sinus opacification (LMK score), difficulty with sense of smell (DSS score), total symptom score (TSS score), percentage of participants requiring systemic corticosteroids or nasal polyp surgery, and time to first such interventions up to week 24.

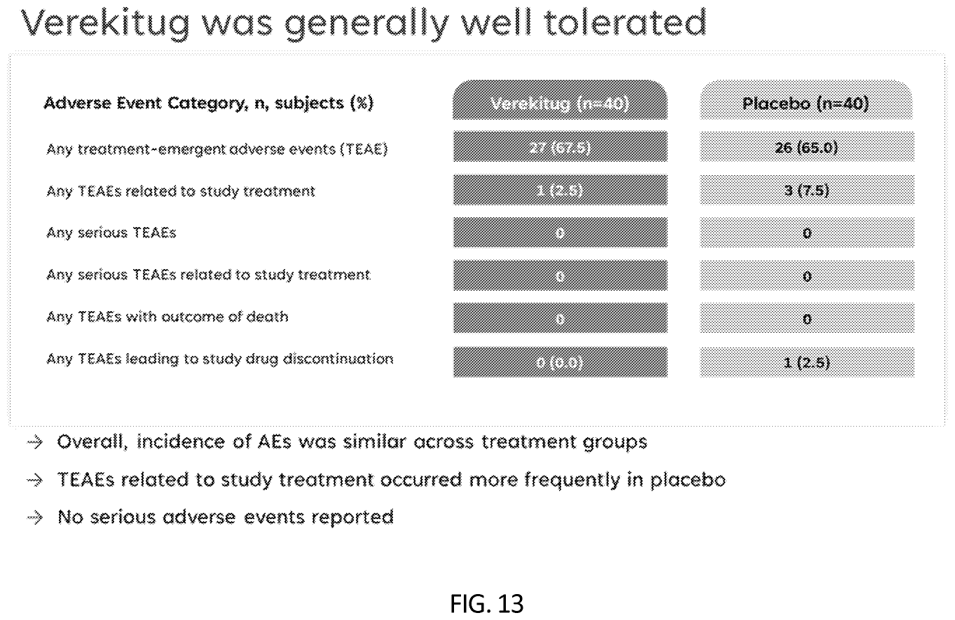

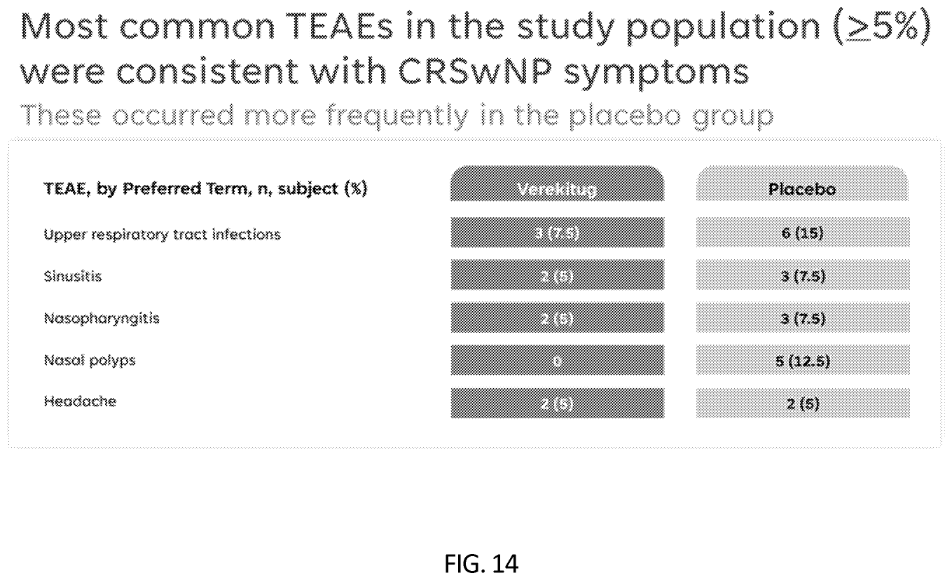

Verekitug was generally well tolerated, demonstrating a favorable safety profile consistent with previous studies, with no SAEs observed (FIGS. 13 and 14).

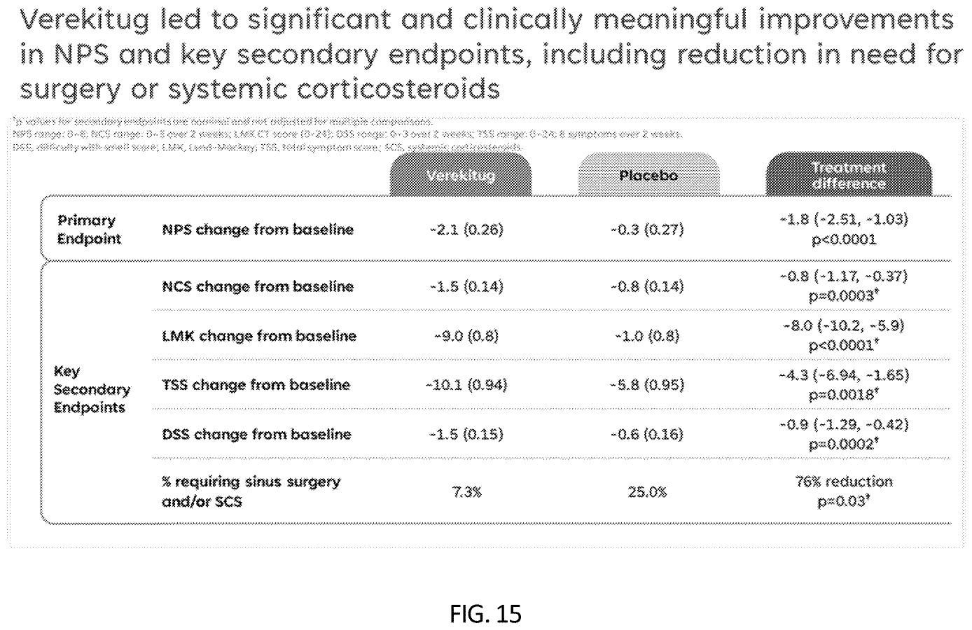

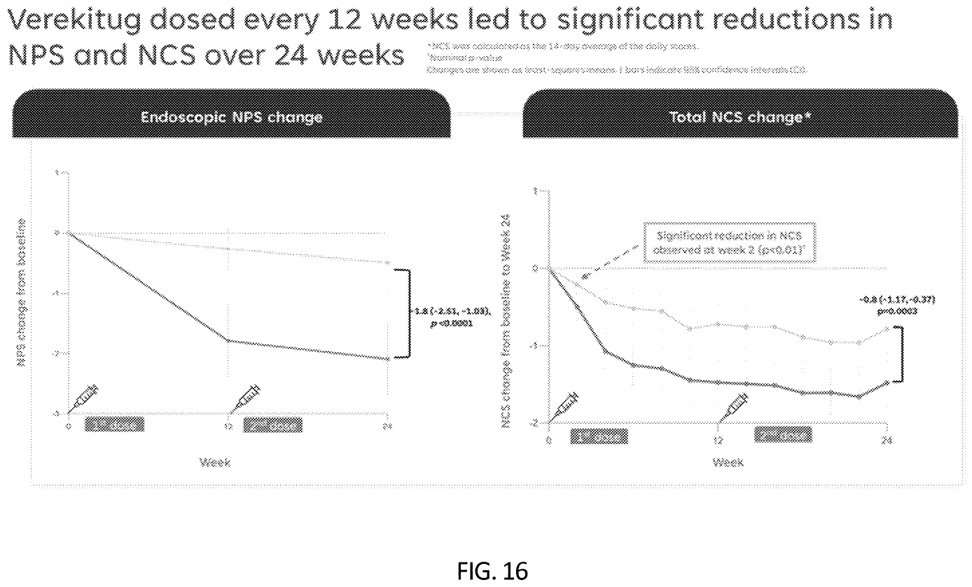

The trial met its primary endpoint, demonstrating clinically meaningful placebo-adjusted reduction in NPS of −1.8 (p<0.0001) at Week 24 compared with baseline. The trial also showed a clinically meaningful placebo-adjusted reduction from baseline in the patient-reported NCS, by −0.8 (p=0.0003) (FIGS. 15 and 16).

Significant improvements were also observed in all secondary endpoints, including sinus opacification as measured by the LMK score (placebo-adjusted reduction of −8 (p<0.0001)), TSS score (placebo-adjusted reduction of −4.3 (p=0.0018)), DSS score (placebo-adjusted reduction of −0.9 (p=0.0002)), reduction in the need for either systemic corticosteroids or nasal polyp surgery (76% risk reduction (p=0.03)) (FIG. 15).

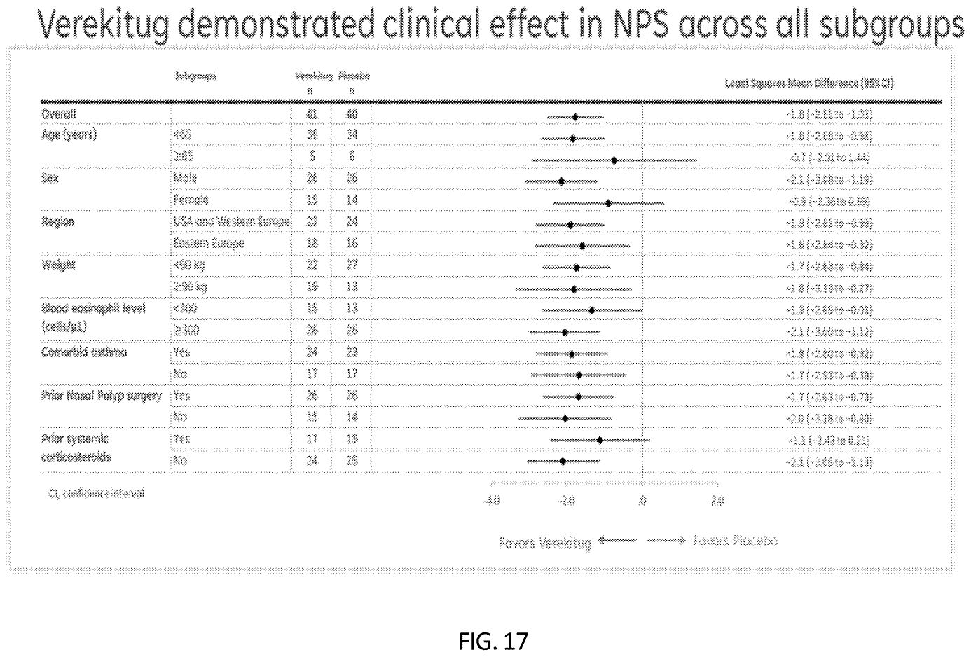

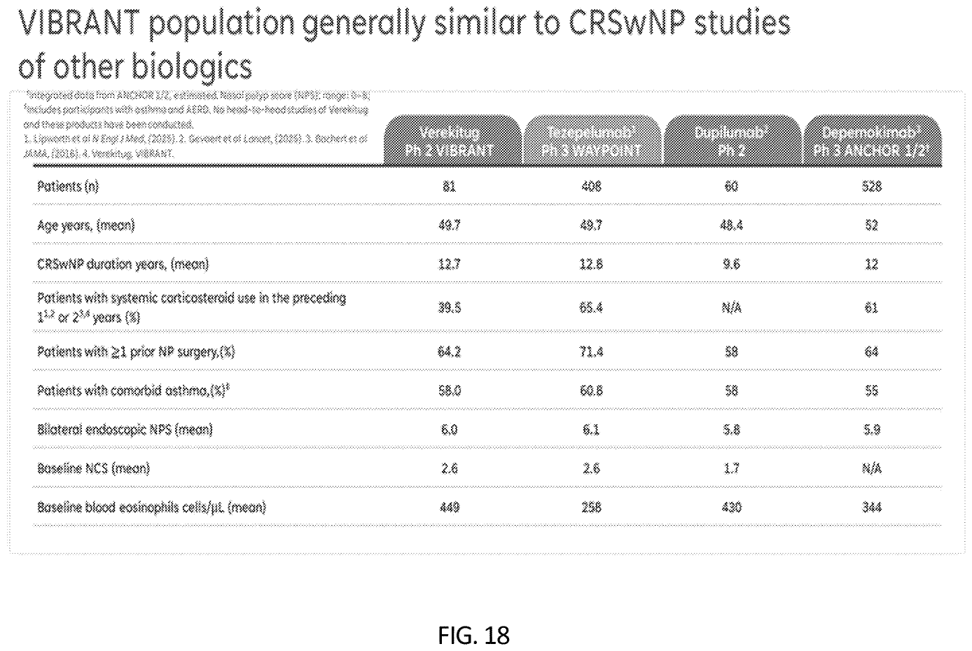

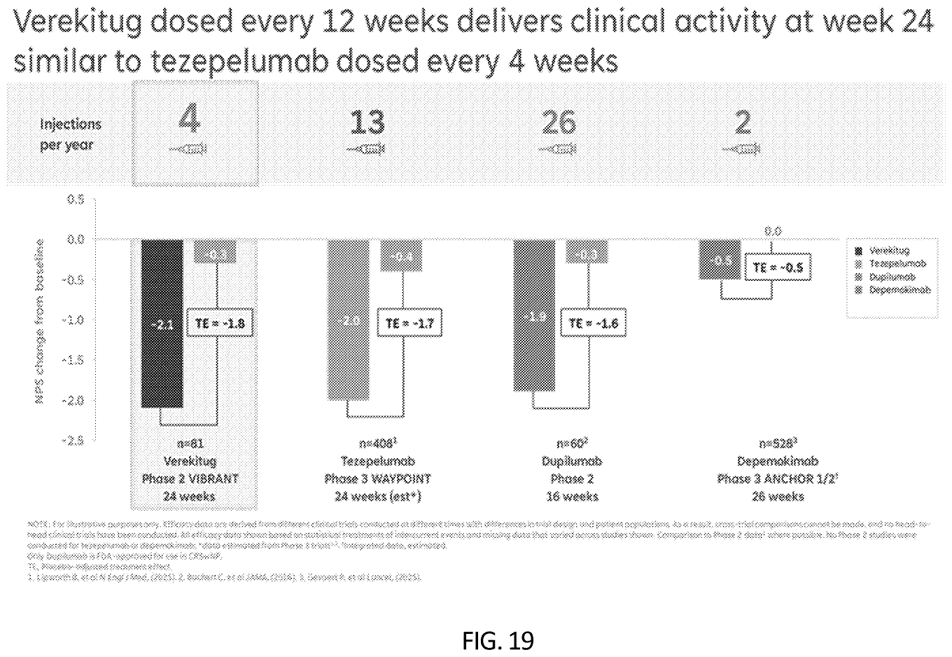

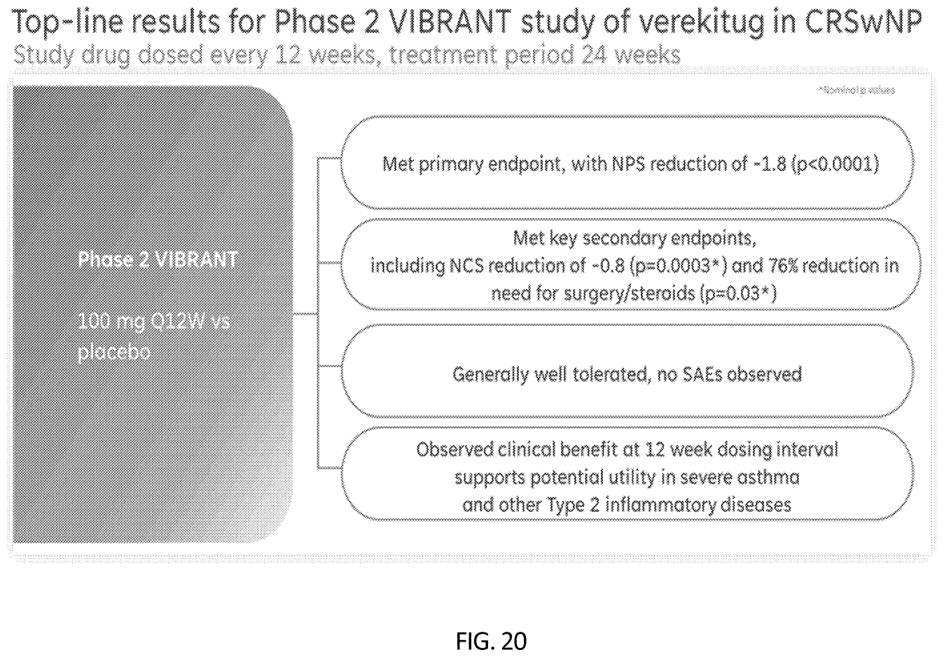

These results demonstrate statistically significant and clinically meaningful benefits with verekitug dosed every 12 weeks in advance the standard of care in the treatment of CRSwNP (FIGS. 17-20).

### Example 7: Phase 2 VIBRANT: Responder Analysis Using Clinically Meaningful Efficacy Outcome Thresholds

Chronic rhinosinusitis with nasal polyps (CRSwNP) is an inflammatory condition associated with high symptom burden and reduced quality of life. Thymic stromal lymphopoietin (TSLP) has emerged as a key driver in CRSwNP pathogenesis, suggesting that the TSLP-signaling pathway is a viable therapeutic target. Verekitug is a highly potent, fully human monoclonal antibody targeting the TSLP receptor. In the VIBRANT trial, verekitug, administered every 12 weeks, demonstrated statistically and clinically significant improvements in nasal polyp score (NPS) and nasal congestion score (NCS) in participants with severe, uncontrolled CRSwNP. This analysis evaluated the effects of verekitug on NPS, NCS, and other symptoms using thresholds of clinically meaningful, within-participant change from baseline.

In this double-blind, placebo-controlled, phase 2 trial, randomized participants with severe, uncontrolled CRSwNP received standard of care and either 100 mg subcutaneous verekitug or matching placebo every 12 weeks for 24 weeks, as outlined above. Responder analyses were performed for participants achieving a ≥1 point decrease at week 24 in NPS, bi-weekly NCS, difficulty with sense of smell (DSS), and nasal blockage score (NBS); ≥4 point decrease in bi-weekly total symptom score (TSS); and ≥5 point decrease in Lund-Mackay score (LMK). The analyses used the worst possible value to account for rescue surgery and employed a worst-observation-carried-forward approach to account for intercurrent use of systemic glucocorticoids for CRSwNP or for comorbid conditions during the study treatment period.

Eighty-one participants (verekitug, n=41; placebo, n=40) were randomized. As shows in the Table 13 below, responder rates were higher with verekitug versus placebo for NPS (79.5% vs 24.3%; odds ratio [OR], 12.06 [95% confidence interval, 4.09-35.53]; P<0.0001), NCS (72.2% vs 39.5%; OR, 3.99; P=0.0046); DSS (69.4% vs 21.1%; OR, 8.52; P<0.0001); NBS (80.6% vs 39.5%; OR, 6.35; P=0.0003); TSS (83.3% vs 36.8%; OR, 8.57; P<0.0001); and LMK (75.7% vs 12.1%; OR, 22.56; P<0.0001.

TABLE 13

| | Verekitug | Placebo |
|---|---|---|
| | (n = 41) | (n = 40) |
| NPS (responder: ≥1 point decrease)^{a} | | |
| Responders, n (%) | 31 (79.5) | 9 (24.3) |
| Nonresponders, n (%) | 8 (20.5) | 28 (75.7) |
| OR of proportion of responders (95% CI) | 12.06 (4.09-35.53) | |
| P value | <0.0001 | |
| NCS (responder: ≥1 point decrease)^{b} | | |
| Responders, n (%) | 26 (72.2) | 15 (39.5) |
| Nonresponders, n (%) | 10 (27.8) | 23 (60.5) |
| OR of proportion of responders (95% CI) | 3.99 (1.50-10.59) | |
| P value | 0.0046 | |
| DSS (responder: ≥1 point decrease)^{b} | | |
| Responders, n (%) | 25 (69.4) | 8 (21.1) |
| Nonresponders, n (%) | 11 (30.6) | 30 (78.9) |
| OR of proportion of responders (95% CI) | 8.52 (2.97-24.45) | |
| P value | <0.0001 | |
| NBS (responder: ≥1 point decrease)^{b} | | |
| Responders, n (%) | 29 (80.6) | 15 (39.5) |
| Nonresponders, n (%) | 7 (19.4) | 23 (60.5) |
| OR of proportion of responders (95% CI) | 6.35 (2.22-18.17) | |
| P value | 0.0003 | |
| TSS (responder: ≥4 point decrease)^{b} | | |
| Responders, n (%) | 30 (83.3) | 14 (36.8) |
| Nonresponders, n (%) | 6 (16.7) | 24 (63.2) |
| OR of proportion of responders (95% CI) | 8.57 (2.86-25.67) | |
| P value | <0.0001 | |
| LMK (responder: ≥5 point decrease) | | |
| Responders, n (%) | 28 (75.7) | 4 (12.1) |
| Nonresponders, n (%) | 9 (24.3) | 29 (87.9) |

| | |
|---|---|
| OR of proportion of responders (95% CI) | 22.56 (6.23-81.71) |
| P value | <0.0001 |

^{a}Verekitug, n = 39; placebo, n = 37.
^{b}Verekitug, n = 36; placebo, n = 38.
^{c}Verekitug, n = 37; placebo, n = 33.
CI, confidence interval;
DSS, difficulty with sense of smell;
LMK, Lund-Mackay score;
NBS, nasal blockage score;
NCS, nasal congestion score;
NPS, nasal polyp score;
OR, odds ratio;
TSS, total symptom score.

### Example 8: Phase 2 VIBRANT: Responder Analysis Using Clinically Meaningful Efficacy Outcome Thresholds

Chronic rhinosinusitis with nasal polyps (CRSwNP) is an inflammatory condition associated with high symptom burden and reduced quality of life. Thymic stromal lymphopoietin (TSLP) has emerged as a key driver in CRSwNP pathogenesis, suggesting that the TSLP-signaling pathway is a viable therapeutic target. Verekitug is a highly potent, fully human monoclonal antibody targeting the TSLP receptor. In the VIBRANT trial, verekitug dosed every 12 weeks in participants with inadequately controlled CRSwNP led to significant improvements in nasal polyp size, sinonasal symptoms, and need for nasal polyp surgery or systemic corticosteroids at week 24. This example presents the baseline participant characteristics and the detailed primary and key secondary efficacy endpoint results from the VIBRANT trial.

In this double-blind, placebo-controlled, phase 2 trial, randomized participants with severe, uncontrolled CRSwNP received standard of care and either 100 mg subcutaneous verekitug or matching placebo every 12 weeks for 24 weeks. Key inclusion criteria were: age 18-85 years; history of NP surgery or NP exacerbation requiring systemic corticosteroids (SCS) in the past 24 months; endoscopic nasal polyp score (NPS)≥5 nasal congestion score (NCS) 2 over 14 days; CRSwNP symptoms for ≥8 weeks; and stable standard of care treatment for CRSwNP for ≥30 days. The primary endpoint was change from baseline in endoscopic NPS at week 24. Key secondary endpoints included change from baseline at week 24 in nasal congestion score (NCS), total symptom score (TSS), difficulty with sense of smell (DSS), and Lund-Mackay score (LMVIK), as well as the proportion of participants requiring nasal polyp surgery or systemic corticosteroids by week 24. In addition to the primary analysis, efficacy endpoints were analyzed using a worst-observation-carried-forward (WOCF) approach to account for use of rescue nasal polyp surgery or systemic corticosteroids, or escalation of maintenance/background treatment for CRSwNP.

Eighty-one participants were randomized (verekitug, n=41; placebo, n=40); baseline participant characteristics were generally balanced between the treatment groups, as shown in Table 14 below.

TABLE 14

| | Verekitug | Placebo | Total |
|---|---|---|---|
| | (n = 41) | (n = 40) | (N = 81) |
| | 2.6 | 2.6 | 2.6 |
| Median age (range), | 50.0 (19-75) | 50.0 (21-76) | 50.0 (19-76) |
| years | | | |
| Male assigned at birth, n | 26 (63.4) | 26 (65.0) | 52 (64.2) |
| (%) | | | |
| Blood eosinophil count, | 404 (260.4) | 496 (378.1)^{a} | 449 (324.4)^{a} |
| mean (SD), cells/μL | | | |
| History of CRSwNP | 26 (63.4) | 26 (65.0) | 52 (64.2) |
| surgery, n (%) | | | |
| History of SCS use, n | 18 (43.9) | 16 (40.0) | 34 (42.0) |
| History of comorbid | 24 (58.5) | 23 (57.5) | 47 (58.0) |
| asthma and/or NSAID- | | | |
| ERD, n (%) | | | |
| Nasal polyp score, mean | 5.90 (1.62) | 6.13 (1.30) | 6.01 (1.47) |
| (SD) | | | |
| Nasal congestion score, | 2.60 (0.43) | 2.67 (0.44) | — |
| mean (SD) | | | |
| Total symptom score, | 17.52 (4.40) | 18.23 (5.11) | — |
| mean (SD) | | | |
| Difficulty with sense of | 2.85 (0.33) | 2.75 (0.57) | — |
| smell, mean (SD) | | | |
| Lund-Mackay score, | 17.13 (4.84)^{b} | 16.86 (4.92) | — |
| mean (SD) | | | |

^{a}n =39.
^{b}n = 36.
CRSwNP, chronic rhinosinusitis with nasal polyps;
NSAID-ERD, nonsteroidal anti-inflammatory drug-exacerbated respiratory disease;
SCS, systemic corticosteroids;
SD, standard deviation.

Verekitug led to significant and clinically meaningful improvements in nasal polyp score (NPS) at week 12, which continued through week 24. The primary endpoint results and key secondary endpoint results are shown in Tables 15 and 16 below.

TABLE 15

| | Primary | | | |
|---|---|---|---|---|
| | analysis | | | |
| | (treatment | | WOCF | |
| | policy) | | analysis | |
| | 2.8 | | 2.8 | |
| | Verekitug | Placebo | Verekitug | Placebo |
| | (n = 41) | (n = 40) | (n = 41) | (n = 40) |
| CFB^{a} at week 24 | −2.05 (0.26)^{b} | −0.28 (0.27)^{c} | −1.97 (0.26)^{d} | −0.02 (0.26)^{e} |
| (SE) | | | | |
| Differencef (95% | −1.77 (−2.51 | | −1.95 (−2.66 | |
| CI), verekitug vs | to −1.03); | | to −1.24); | |
| placebo | P < 0.0001 | | P < 0.0001 | |

^{a}All CFB are LSM.
^{b}n = 39.
^{c}n = 35.
^{d}n = 37.
^{e}n = 37.
^{f}LSM difference.
CFB, change from baseline;
CI, confidence interval;
LSM, least squares mean;
NPS, nasal polyp score;
SE, standard error;
WOCF, worst observation carried forward.

TABLE 16

| | | | |
|---|---|---|---|
| Primary analysis | | WOCF analysis | |
| (treatment policy) | | 2.9 | |
| 2.9 | Placebo | Verekitug | Placebo |
| Verekitug (n = 41) | (n = 40) | (n = 41) | (n = 40) |

Nasal congestion score

| | | | | |
|---|---|---|---|---|
| CFB^{a} at week 24 | −1.53 (0.14)^{b} | −0.76 (0.14)^{c} | −1.48 (0.15)^{d} | −0.52 (0.15)^{e} |
| (SE) | | | | |
| Difference^{f} (95% | −0.77 (−1.17 to | | −0.96 (−1.37 to | |
| CI), verekitug vs | −0.37); | | −0.55); | |
| placebo | P = 0.0003 | | P < 0.0001 | |

Total symptom score

| | | | | |
|---|---|---|---|---|
| CFB^{a} at week 24 | −10.18 (0.97)^{b} | −5.49 (0.98)^{c} | −9.88 (1.02)^{d} | −3.54 (1.02)^{e} |
| (SE) | | | | |
| Difference^{f} (95% | −4.69 (−7.41 to | | −6.34 (−9.19 to | |
| CI), verekitug vs | −1.96); | | −3.49); | |
| placebo | P = 0.0010 | | P < 0.0001 | |

Difficulty with sense of smell

| | | | | |
|---|---|---|---|---|
| CFB^{a} at week 24 | −1.47 (0.15)^{b} | −0.62 (0.16)^{c} | −1.46 (0.14)^{d} | −0.37 (0.15)^{e} |
| (SE) | | | | |
| Difference^{f} (95% | −0.85 (−1.29 to | | −1.08 (−1.49 to | |
| CI), verekitug vs | −0.42); | | −0.68); | |
| placebo | P = 0.0002 | | P < 0.0001 | |

Lund-Mackay score

| | | | | |
|---|---|---|---|---|
| CFB^{a} at week 24 | −9.45 (0.75)^{b} | −1.38 (0.78)^{c} | −9.56 (0.71)^{d} | −1.35 (0.74)^{e} |
| (SE) | | | | |
| Difference^{f} (95% | −8.07 (−10.23 to | | −8.22 (−10.26 to | |
| CI), verekitug vs | −5.92); | | −6.18); | |
| placebo | P < 0.0001 | | P < 0.0001 | |

Proportion of participants requiring NP surgery and/or SCS at week 24

| | | | | |
|---|---|---|---|---|
| Participants, n (%) | 3 (7.3)^{g} | 10 (25.0)^{h} | — | — |
| Odds ratio | 0.24 (0.06-0.94); | | — | — |
| (95% CI) | P = 0.03 | | | |

^{a}All CFB are LSM.
^{b}n = 36.
^{c}n = 35.
^{d}n = 38.
^{e}n = 33.
^{f}LSM difference.
^{g}All 3 participants required SCS, but not NP surgery.
^{h}Nine participants required SCS and 1 required NP surgery.
CFB, change from baseline;
CI, confidence interval;
LSM, least squares mean;
NP, nasal polyp;
SCS, systemic corticosteroids;
SE, standard error;
WOCF, worst observation carried forward.

Verekitug led to significant improvements in nasal congestion score (NCS) as early as week 2 and continued through week 24. A significant reduction in NCS was observed at week 2 (P<0.01). Verekitug reduced the need for surgery or systemic corticosteroids (SCS) by 76% compared with placebo. Significant improvements were observed in all other secondary endpoints at week 24. Accordingly, participants with CRSwNP treated with verekitug 100 mg subcutaneously every 12 weeks demonstrated statistically significant and clinically meaningful improvements across all primary and secondary efficacy endpoints, with an early onset of nasal congestion improvement detectable as early as week 2 from the first dose.

Accordingly, participants with CRSwNP treated with verekitug compared to placebo demonstrated clinically meaningful improvements in objective and patient-reported outcomes.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications in addition to those described herein will become apparent to those skilled in the art from the foregoing description. Such modifications are intended to fall within the scope of the embodiments and any appended claims.

The present specification is considered to be sufficient to enable one skilled in the art to practice the embodiments. Various modifications in addition to those shown and described herein will become apparent to those skilled in the art from the foregoing description and fall within the scope of the present disclosure and any appended claims.

### ENUMERATED EMBODIMENTS

1. A method of treating Chronic Rhinosinusitis with Nasal Polyps in a subject in need thereof, comprising administering to the subject a therapeutically effective amount of an anti-TSLP-R antibody, or antigen-binding fragment thereof.
2. The method of embodiment 1, wherein the anti-TSLP-R antibody, or antigen-binding fragment thereof, comprises:
   (i) a heavy chain variable region comprising an HCDR1 amino acid sequence, an HCDR2 amino acid sequence, and an HCDR3 amino acid sequence, wherein the HCDR1 has the amino acid sequence of SEQ ID NO: 7; the HCDR2 has the amino acid sequence of SEQ ID NO: 8; and the HCDR3 sequence has the amino acid sequence of SEQ ID NO: 9; or variants of any of the foregoing; and
   (ii) a light chain variable region comprising a LCDR1 amino acid sequence, a LCDR2 amino acid sequence, and a LCDR3 amino acid sequence, wherein the LCDR1 has the amino acid sequence SEQ ID NO: 10; the LCDR2 has the amino acid sequence of SEQ ID NO: 11; and the LCDR3 has the amino acid sequence of SEQ ID NO: 12; or variants of any of the foregoing.
3. The method of any one of embodiments 1-2, wherein the anti-TSLP-R antibody, or antigen-binding fragment thereof, comprises a heavy chain variable region having a sequence that is at least 90%, 91%, 92%, 93%, 94%, 95%, 96%, 97%, 98%, or 99% identical to the sequence of SEQ ID NO: 5.
4. The method of any one of embodiments 1-3, wherein the anti-TSLP-R antibody, or antigen-binding fragment thereof, comprises a heavy chain variable region having a sequence that is at least 90%, 91%, 92%, 93%, 94%, 95%, 96%, 97%, 98%, or 99% identical to the sequence of SEQ ID NO: 5, provided that the CDRs comprise the HCDR1 amino acid sequence of SEQ ID NO: 7; the HCDR2 amino acid sequence of SEQ ID NO: 8; and the HCDR3 amino acid sequence of SEQ ID NO: 9.
5. The method of any one of embodiments 1-4, wherein the anti-TSLP-R antibody, or antigen-binding fragment thereof, comprises a light chain variable region having a sequence that is at least 90%, 91%, 92%, 93%, 94%, 95%, 96%, 97%, 98%, or 99% identical to the sequence of SEQ ID NO: 6.
6. The method of any one of embodiments 1-5, wherein the anti-TSLP-R antibody, or antigen-binding fragment thereof, comprises a light chain variable region having a sequence that is at least 90%, 91%, 92%, 93%, 94%, 95%, 96%, 97%, 98%, or 99% identical to the sequence of SEQ ID NO: 6, provided that the CDRs comprise the LCDR1 amino acid sequence of SEQ ID NO: 10; the LCDR2 amino acid sequence of SEQ ID NO: 11; and the LCDR3 amino acid sequence of SEQ ID NO: 12.
7. The method of any one of embodiments 1-6, wherein the anti-TSLP-R antibody, or antigen-binding fragment thereof, comprises:
   (i) a heavy chain variable region having a sequence that is at least 90%, 91%, 92%, 93%, 94%, 95%, 96%, 97%, 98%, or 99% identical to the amino acid sequence of SEQ ID NO: 5; and
   (ii) a light chain variable region having a sequence that is at least 90%, 91%, 92%, 93%, 94%, 95%, 96%, 97%, 98%, or 99% identical to the amino acid sequence of SEQ ID NO: 6.
8. The method of any one of embodiments 1-7, wherein the anti-TSLP-R antibody, or antigen-binding fragment thereof, comprises:
   (i) a heavy chain variable region having a sequence that is at least 90%, 91%, 92%, 93%, 94%, 95%, 96%, 97%, 98%, or 99% identical to the sequence of SEQ ID NO: 5, provided that the CDRs comprise the HCDR1 amino acid sequence of SEQ ID NO: 7; the HCDR2 amino acid sequence of SEQ ID NO: 8; and the HCDR3 amino acid sequence of SEQ ID NO: 9; and
   (ii) a light chain variable region having a sequence that is at least 90%, 91%, 92%, 93%, 94%, 95%, 96%, 97%, 98%, or 99% identical to the sequence of SEQ ID NO: 6, provided that the CDRs comprise the LCDR1 amino acid sequence of SEQ ID NO: 10; the LCDR2 amino acid sequence of SEQ ID NO: 11; and the LCDR3 amino acid sequence of SEQ ID NO: 12.
9. The method of any one of embodiments 1-8, wherein the anti-TSLP-R antibody, or antigen-binding fragment thereof, comprises a heavy chain variable region having a sequence of SEQ ID NO: 5.
10. The method of any one of embodiments 1-9, wherein the anti-TSLP-R antibody, or antigen-binding fragment thereof, comprises a light chain variable region having a sequence of SEQ ID NO: 6.
11. The method of any one of embodiments 1-10, wherein the anti-TSLP-R antibody, or antigen-binding fragment thereof, comprises:
   (i) a heavy chain variable region having a sequence of SEQ ID NO: 5; and
   (ii) a light chain variable region having a sequence of SEQ ID NO: 6.
12. The method of any one of embodiments 1-11, wherein the anti-TSLP-R antibody, or antigen-binding fragment thereof, comprises a heavy chain having a sequence that is at least 90%, 91%, 92%, 93%, 94%, 95%, 96%, 97%, 98%, or 99% identical to the sequence of SEQ ID NO: 1.
13. The method of any one of embodiments 1-12, wherein the anti-TSLP-R antibody, or antigen-binding fragment thereof, comprises a light chain having a sequence that is at least 90%, 91%, 92%, 93%, 94%, 95%, 96%, 97%, 98%, or 99% identical to the sequence of SEQ ID NO: 3.
14. The method of any one of embodiments 1-13, wherein the anti-TSLP-R antibody, or antigen-binding fragment thereof, comprises a heavy chain having a sequence of SEQ ID NO: 1.
15. The method of any one of embodiments 1-14, wherein the anti-TSLP-R antibody, or antigen-binding fragment thereof, comprises a light chain having a sequence of SEQ ID NO: 3.
16. The method of any one of embodiments 1-15, wherein the anti-TSLP-R antibody, or antigen-binding fragment thereof, comprises:
   (i) a heavy chain having a sequence that is at least 90%, 91%, 92%, 93%, 94%, 95%, 96%, 97%, 98%, or 99% identical to the sequence of SEQ ID NO: 1; and
   (ii) a light chain having a sequence that is at least 90%, 91%, 92%, 93%, 94%, 95%, 96%, 97%, 98%, or 99% identical to the sequence of SEQ ID NO: 3.
17. The method of any one of embodiments 1-16, wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3.
18. The method of any one of embodiments 1-17, wherein the anti-TSLP-R antibody, or antigen-binding fragment thereof, is administered to the subject by injection.
19. The method of embodiment 18, wherein the injection is an intraarterial injection, an intraarticular injection, an intracameral injection, an intracerebral injection, an intradermal injection, an intramedullary injection, an intramuscular injection, an intraocular/intravitreal injection, an intraperitoneal injection, an intrathecal/intraspinal injection, an intravenous injection, an intraventricular injection, a subcutaneous injection, a suprachoroidal injection, or a retroorbital injection.
20. The method of any one of embodiments 1-19, wherein the anti-TSLP-R antibody, or antigen-binding fragment thereof, is administered to the subject subcutaneously.
21. The method of any one of embodiments 1-20, wherein the anti-TSLP-R antibody, or antigen-binding fragment thereof, is administered to the subject intravenously.
22. The method of any one of embodiments 1-21, wherein the anti-TSLP-R antibody, or antigen-binding fragment thereof, is administered to the subject at least once every 1 day, once every 1 week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, once every 26 weeks, once every 27 weeks, once every 28 weeks, once every 29 weeks, once every 30 weeks, once every 31 weeks, once every 32 weeks, once every 33 weeks, once every 34 weeks, once every 35 weeks, once every 36 weeks, once every 37 weeks, once every 38 weeks, once every 39 weeks, once every 40 weeks, once every 41 weeks, once every 42 weeks, once every 43 weeks, once every 44 weeks, once every 45 weeks, once every 46 weeks, once every 47 weeks, once every 48 weeks, once every 49 weeks, once every 50 weeks, once every 51 weeks, once every 52 weeks, once every 1 month, once every 2 months, once every 3 months, once every 4 months, once every 5 months, once every 6 months, once every 7 months, once every 8 months, once every 9 months, once every 10 months, once every 11 months, or once every 12 months.
23. The method of any one of embodiments 1-22, wherein the therapeutically effective amount of the anti-TSLP-R antibody, or antigen-binding fragment thereof, is about 1.0 mg/kg to about 15.0 mg/kg.
24. The method of any one of embodiments 1-23, wherein the therapeutically effective amount of the anti-TSLP-R antibody, or antigen-binding fragment thereof, is administered as an initial dose then a maintenance dose.
25. The method of embodiment 24, wherein the initial dose is about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10.0 mg/kg, about 10.5 mg/kg, about 11.0 mg/kg, about 11.5 mg/kg, about 12.0 mg/kg, about 12.5 mg/kg, about 13.0 mg/kg, about 13.5 mg/kg, about 14.0 mg/kg, about 14.5 mg/kg, or about 15.0 mg/kg.
26. The method of embodiment 24, wherein the maintenance dose is about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, about 10.0 mg/kg, about 10.5 mg/kg, about 11.0 mg/kg, about 11.5 mg/kg, about 12.0 mg/kg, about 12.5 mg/kg, about 13.0 mg/kg, about 13.5 mg/kg, about 14.0 mg/kg, about 14.5 mg/kg, or about 15.0 mg/kg.
27. The method of embodiment 24, wherein the initial dose is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg.
28. The method of embodiment 24, wherein the maintenance dose is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg.
29. The method of any one of embodiments 1-28, wherein the anti-TSLP-R antibody, or antigen-binding fragment thereof, is administered at about 20 mg, 25 mg, 50 mg, 100 mg, 150 mg, 200 mg, 250 mg, 300 mg, or 400 mg every week.
30. The method of any one of embodiments 1-29, wherein the anti-TSLP-R antibody, or antigen-binding fragment thereof, is administered at about 20 mg, 25 mg, 50 mg, 100 mg, 150 mg, 200 mg, 250 mg, 300 mg, or 400 mg every 2 weeks.
31. The method of any one of embodiments 1-30, wherein the anti-TSLP-R antibody, or antigen-binding fragment thereof, is administered at about 20 mg, 25 mg, 50 mg, 100 mg, 150 mg, 200 mg, 250 mg, 300 mg, or 400 mg every 4 weeks.
32. The method of any one of embodiments 1-31, wherein the anti-TSLP-R antibody, or antigen-binding fragment thereof, is administered at about 20 mg, 25 mg, 50 mg, 100 mg, 150 mg, 200 mg, 250 mg, 300 mg, or 400 mg every 8 weeks.
33. The method of any one of embodiments 1-32, wherein the anti-TSLP-R antibody, or antigen-binding fragment thereof, is administered at about 20 mg, 25 mg, 50 mg, 100 mg, 150 mg, 200 mg, 250 mg, 300 mg, or 400 mg every 12 weeks.
34. The method of embodiment 33, wherein the anti-TSLP-R antibody, or antigen-binding fragment thereof, is administered at 100 mg every 12 weeks.
35. The method of any one of embodiments 1-34, wherein the anti-TSLP-R antibody, or antigen-binding fragment thereof, is administered at about 100 mg, 200 mg, 250 mg, 300 mg, 350 mg, 400 mg, 500 mg, 600 mg, 700 mg, or 800 mg every 16 weeks.
36. The method of any one of embodiments 1-35, wherein the anti-TSLP-R antibody, or antigen-binding fragment thereof, is administered at about 100 mg, 200 mg, 250 mg, 300 mg, 350 mg, 400 mg, 500 mg, 600 mg, 700 mg, or 800 mg every 24 weeks.
37. The method of any one of embodiments 1-36, wherein the anti-TSLP-R antibody, or antigen-binding fragment thereof, is administered for a total of 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks.
38. The method of any one of embodiments 1-37, wherein the subject has an improvement of one or more symptom of CRSwNP following administration of the anti-TSLP-R antibody, or antigen-binding fragment thereof.
39. The method of any one of embodiments 1-38, wherein the one or more symptom of CRSwNP is improved after, or about, 1 week, 2 weeks, 3 weeks, 4 weeks, 5 weeks, 6 weeks, 7 weeks, 8 weeks, 9 weeks, 10 weeks of treatment with the anti-TSLP-R antibody, or antigen-binding fragment thereof.
40. The method of any one of embodiments 1-39, wherein the one or more symptom of CRSwNP continues to improve after administration of the anti-TSLP-R antibody, or antigen-binding fragment thereof, has ceased for at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks.
41. The method of any one of embodiments 1-40, wherein the anti-TSLP-R antibody, or antigen-binding fragment thereof, is co-administered to the subject with a second therapeutic agent.
42. The method of embodiment 41, wherein the second therapeutic agent is co-administered by injection.
43. The method of any one of embodiments 41-42, wherein the second therapeutic is co-administered at the same time as, prior to, or following administration of the anti-TSLP-R antibody, or antigen-binding fragment thereof.
44. The method of any one of embodiments 41-43, wherein the second therapeutic agent is selected from levofloxacin, ciprofloxacin, moxifloxacin, sulfamethoxazole/trimethoprim, acetaminophen/caffeine, amoxicillin, ampicillin, oxacillin, amoxicillin/clavulante, ampicillin/sulbactam, chlorpheniramine/pseudoephedrine, dexchlorpheniramine/dextromethorphan/pseudoephedrine, diphenhydramine/phenylephrine, dextromethorphan/diphenhydramine/phenylephrine, dextromethorphan/phenylephrine/pyrilamine, cefuroxime, cefprozil, cefaclor, cefuroxime, cefprozil, cefaclor, cefdinir, cefixime, cefpodoxime, ceftazidime, clindamycin, prednisone, fluticasone, mometasone, beclomethasone, azithromycin, clarithromycin, omalizumab, dupilumab, mepolizumab, or any combination thereof.
45. The method of any one of embodiments 41-44, wherein the second therapeutic agent is an oral corticosteroid or an intranasal corticosteroid.
46. The method of any one of embodiments 41-45, wherein the second therapeutic agent is an intranasal corticosteroid that comprises mometasone furoate.
47. The method of any one of embodiments 41-45, wherein the second therapeutic agent is administered as a nasal spray.
48. The method of any one of embodiments 1-47, wherein the subject has a history of inadequate response to treatment with intranasal corticosteroids.
49. The method of any one of embodiments 1-48, wherein the subject has one or more nasal polyp present in each nostril prior to the administering of the anti-TSLP-R antibody, or antigen-binding fragment thereof.
50. The method of any one of embodiments 1-49, wherein the subject has a total of at least 5 nasal polyps, with two or more nasal polyps present in each nostril prior to the administering of the anti-TSLP-R antibody, or antigen-binding fragment thereof.
51. The method of any one of embodiments 1-50, wherein the subject has previously had a relapse of nasal polyposis after receiving surgery to treat the polyps and prior to the administering of the anti-TSLP-R antibody, or antigen-binding fragment thereof.
52. The method of any one of embodiments 1-51, wherein the subject achieves an improvement of one or more symptom of CRSwNP following administration of the anti-TSLP-R antibody, or antigen-binding fragment thereof.
53. The method of any one of embodiments 1-52, wherein the subject achieves an improvement of one or more symptom of CRSwNP chosen from loss of smell, nasal congestion, anterior nasal discharge, posterior nasal discharge, nasal peak inspiratory flow (NPIF) that occur in the day (AM), nasal peak inspiratory flow (NPIF) that occur in the night (PM), facial pain, headache, or any combination thereof, following administration of the anti-TSLP-R antibody, or antigen-binding fragment thereof.
54. The method of any one of embodiments 1-53, wherein the subject achieves an improvement of one or more symptom of CRSwNP as measured by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, patient reported outcomes, or any combination thereof, compared to baseline.
55. The method of embodiment 54, wherein the subject achieves an improvement of one or more patient reported outcomes according to one or more assessment chosen from the Short-Form-36 (SF-36) Questionnaire, the Eurogol-5D (EQ-5D), nasal polyp related resource use questionnaire, the patient qualitative self-assessment, or any combination thereof, compared to baseline.
56. The method of any one of embodiments 1-55, wherein the subject achieves an improvement of a nasal polyp-associated biomarker, compared to baseline.
57. The method of embodiment 56, wherein the subject achieves an improvement of a nasal polyp-associated biomarker chosen from IgE, TARC, eotaxin-3 and ECP, compared to baseline.
58. The method of any one of embodiments 1-57, wherein the subject achieves a reduction in the size of one or more nasal polyp as assessed by the nasal polyp score (NPS) compared to baseline.
59. The method of any one of embodiments 1-58, wherein the subject achieves a reduction in the number of nasal polyps present compared to baseline.
60. The method of any one of embodiments 1-59, wherein the need for treatment with intranasal or oral corticosteroids is reduced in the subject.
61. The method of any one of embodiments 1-60, wherein the need for treatment with nasal surgery is reduced in the subject.
62. A method of treating Chronic Rhinosinusitis with Nasal Polyps in a subject in need thereof, the method comprising administering to the subject about 1.0 mg/kg to about 15.0 mg/kg of an anti-TSLP-R antibody, or antigen-binding fragment thereof,
   wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3,
wherein the subject achieves an improvement in one or more symptom of CRSwNP chosen from loss of smell, nasal congestion, anterior nasal discharge, posterior nasal discharge, nasal peak inspiratory flow (NPIF) that occur in the day (AM), nasal peak inspiratory flow (NPIF) that occur in the night (PM), facial pain, headache, or any combination thereof.
63. A method of treating Chronic Rhinosinusitis with Nasal Polyps in a subject in need thereof, the method comprising administering to the subject about 1.0 mg/kg to about 15.0 mg/kg of an anti-TSLP-R antibody, or antigen-binding fragment thereof,
   wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3,
wherein the subject achieves an improvement of one or more symptom of CRSwNP as measured by an assessment chosen from the endoscopic Nasal Polyp Score (NPS); Nasal Congestion Score (NCS); Sinus Opacification Score (LMK); Total Symptom Score (TSS); Difficulty With Smell Score (DSS); Nasal Blockage Score (NBS); the 22-item Sino Nasal Outcome Test (SNOT-22) score; subject-assessed nasal congestion/obstruction, anterior rhinorrhea (runny nose), posterior rhinorrhea (post nasal drip) and loss of sense of smell; number of nocturnal awakenings; Visual Analog Score (VAS) to assess patient-rated rhinosinusitis symptom severity; five-item Asthma Control Questionnaire (ACQ5) score, such as in patients with asthma; Nasal Peak Inspiratory Flow (NPIF); smell test (University of Pennsylvania Smell Identification Test (UPSIT)); spirometry; physiological parameters, such as measured by nasal endoscopy and CT scan; Lund-Mackay Score; Three Dimensional volumetric measurement of the maxillary sinus, patient reported outcomes, or any combination thereof, compared to baseline.
64. A method of treating Chronic Rhinosinusitis with Nasal Polyps in a subject in need thereof, the method comprising administering to the subject about 1.0 mg/kg to about 15.0 mg/kg of an anti-TSLP-R antibody, or antigen-binding fragment thereof,
   wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3,
wherein the subject achieves an improvement of one or more patient reported outcomes according to one or more assessment chosen from the Short-Form-36 (SF-36) Questionnaire, the Eurogol-5D (EQ-5D), nasal polyp related resource use questionnaire, the patient qualitative self-assessment, or any combination thereof, compared to baseline.
65. A method of treating Chronic Rhinosinusitis with Nasal Polyps in a subject in need thereof, the method comprising administering to the subject about 1.0 mg/kg to about 15.0 mg/kg of an anti-TSLP-R antibody, or antigen-binding fragment thereof,
   wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3,
wherein the subject achieves an improvement of a nasal polyp-associated biomarker, compared to baseline.
66. A method of treating Chronic Rhinosinusitis with Nasal Polyps in a subject in need thereof, the method comprising administering to the subject about 1.0 mg/kg to about 15.0 mg/kg of an anti-TSLP-R antibody, or antigen-binding fragment thereof,
   wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3,
wherein the subject achieves an improvement of a nasal polyp-associated biomarker chosen from IgE, TARC, eotaxin-3 and ECP, compared to baseline.
67. A method of treating Chronic Rhinosinusitis with Nasal Polyps in a subject in need thereof, the method comprising administering to the subject about 1.0 mg/kg to about 15.0 mg/kg of an anti-TSLP-R antibody, or antigen-binding fragment thereof,
   wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3,
wherein the subject achieves a reduction in the size of one or more nasal polyp as assessed by the nasal polyp score (NPS) compared to baseline.
68. A method of treating Chronic Rhinosinusitis with Nasal Polyps in a subject in need thereof, the method comprising administering to the subject about 1.0 mg/kg to about 15.0 mg/kg of an anti-TSLP-R antibody, or antigen-binding fragment thereof,
   wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3,
wherein the subject achieves a reduction in the number of nasal polyps present compared to baseline.
69. A method of treating Chronic Rhinosinusitis with Nasal Polyps in a subject in need thereof, the method comprising administering to the subject about 1.0 mg/kg to about 15.0 mg/kg of an anti-TSLP-R antibody, or antigen-binding fragment thereof,
   wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3,
wherein the need for treatment with intranasal or oral corticosteroids is reduced in the subject.
70. A method of treating Chronic Rhinosinusitis with Nasal Polyps in a subject in need thereof, the method comprising administering to the subject about 1.0 mg/kg to about 15.0 mg/kg of an anti-TSLP-R antibody, or antigen-binding fragment thereof,
   wherein the anti-TSLP-R antibody, or antigen-binding fragment thereof, comprises:
   (i) a heavy chain having a sequence of SEQ ID NO: 1; and
   (ii) a light chain having a sequence of SEQ ID NO: 3,
wherein the need for treatment with nasal surgery is reduced in the subject.
71. The method of any one of embodiments 1-70, wherein the subject has a blood eosinophil concentration of at least 200 eosinophiles/μL, 300 eosinophiles/μL, or 400 eosinophiles/μL prior to the administration of the anti-TSLP-R antibody, or antigen-binding fragment thereof.
72. The method of any one of embodiments 1-71, wherein the subject has a decrease in blood eosinophil concentration following the administration of the anti-TSLP-R antibody, or antigen-binding fragment thereof.
73. The method of any one of embodiments 1-72, wherein the subject has a continued decrease in blood eosinophil concentration after administration of anti-TSLP-R antibody, or antigen-binding fragment thereof, has ceased.
74. The method of any one of embodiments 1-73, wherein the subject has a continued decrease in blood eosinophil concentration after at least 1 administration of the anti-TSLP-R antibody, or antigen-binding fragment thereof.
75. The method of embodiment 74, wherein the subject has a continued decrease in blood eosinophil concentration for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, or about 24 weeks after the at least 1 administration of the anti-TSLP-R antibody, or antigen-binding fragment thereof.
76. The method of any one of embodiments 74-75, wherein:
   the anti-TSLP-R antibody, or antigen-binding fragment thereof, is administered to the subject about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, about every 20 weeks, about every 21 weeks, about every 22 weeks, about every 23 weeks, or about every 24 weeks; and
   the subject has a decrease in blood eosinophil concentration following 2, 3, 4, 5, or more administrations of the anti-TSLP-R antibody, or antigen-binding fragment thereof.
77. The method of any one of embodiments 73-76, wherein:
   the anti-TSLP-R antibody, or antigen-binding fragment thereof, is administered to the subject every 4 weeks; and
   the subject has a decrease in blood eosinophil concentration following 3 administrations of the anti-TSLP-R antibody, or antigen-binding fragment thereof.
78. The method of any one of embodiments 73-77, wherein the subject has a decrease in blood eosinophil concentration that is sustained after administration of the anti-TSLP-R antibody, or antigen-binding fragment thereof has ceased for at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks.
79. The method of any one of embodiments 1-78, wherein the subject has a sustained and measurable serum concentration of the anti-TSLP-R antibody, or antigen-binding fragment thereof after administration has ceased for at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks.
80. The method of any one of embodiments 1-79, wherein
   the anti-TSLP-R antibody, or antigen-binding fragment thereof, is administered to the subject about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, about every 20 weeks, about every 21 weeks, about every 22 weeks, about every 23 weeks, or about every 24 weeks;
   at least 2 administrations or at least 3 administrations of the anti-TSLP-R antibody, or antigen-binding fragment thereof, are administered; and
   the subject has a sustained and measurable serum concentration of the anti-TSLP-R antibody, or antigen-binding fragment thereof after administration has ceased for at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks.
81. A method of treating chronic rhinosinusitis with nasal polyps (CRSwNP) in a subject in need thereof, the method comprising:
   administering a pharmaceutical composition comprising about 100 to about 150 mg of verekitug to the subject in need thereof.
82. The method of embodiment 81, wherein the composition is administered once every twelve weeks.
83. The method of embodiments 81 or 82, wherein, the pharmaceutical composition comprises about 100, about 125, or about 150 mg of verekitug.
84. The method of any one of embodiments 1-83, wherein the pharmaceutical composition is administered subcutaneously.
85. The method of any one of embodiments 1-84, wherein verekitug binds to a TSLP receptor (TSLP-R).
86. The method of any one of embodiments 1-85, wherein verekitug blocks the formation of a heterodimeric TSLP receptor (TSLP-R).
87. The method of any one of embodiments 1-86, wherein the subject has a history of nasal polyp (NP) surgery or NP exacerbation requiring steroids (e.g., corticosteroids) being administered to the subject sometime in the 24 months preceding the administration of the pharmaceutical composition.
88. The method of any one of embodiments 1-87, wherein the subject has an endoscopic nasal polyp score (NPS)≥5.
89. The method of any one of embodiments 1-88, wherein the subject has a nasal congestion score (NCS)≥2 over 14 days.
90. The method of any one of embodiments 1-89, wherein the subject has CRSwNP symptoms for at least 8 weeks.
91. The method of any one of embodiments 1-90, wherein the subject in need thereof has had stable standard of care for the treatment of CRSwNP for at least 30 days.
92. The method of any one of embodiments 1-91, wherein the subject in need thereof has asthma.
93. The method of any one of embodiments 1-92, wherein the subject is administered a second dose of verekitug at 12 weeks after the first dose.
94. The method of any one of the preceding embodiments, wherein the reduction in the nasal polyp score (NPS) is at least −1.0, −1.5, −2.0, or −2.5.
95. The method of embodiment 94, wherein the reduction in the nasal polyp score (NPS) is measured at 24 weeks from the first dose.
96. The method of any one of the preceding embodiments, wherein the change of the nasal congestion score (NCS) is a reduction by at least −1.0, −1.5, or −1.75.
97. The method of embodiment 96, wherein the reduction in the nasal congestion score (NCS) is measured at 24 weeks from the first dose.
98. The method of any one of the preceding embodiments, wherein the sinus opacification (LMK score) change from baseline is at least −2, −3, −4, −5, −6, −7, −8, −9, or −10.
99. The method of embodiment 98, wherein the wherein the sinus opacification (LMK score) change from baseline is measured at 24 weeks from the first dose.
100. The method of any one of the preceding embodiments, wherein the total symptom score (TSS) change from baseline is at least −7, −8, −9, −10, or −11.
101. The method of embodiment 100, wherein the TSS change from baseline is measured at 24 weeks from the first dose.
102. The method of any one of the preceding embodiments, wherein the difficulty with smell (DSS score) change from baseline is at least −1, −1.25, or −1.5.
103. The method of any one of the preceding embodiments, wherein the nasal blockage score (NBS) change from baseline is at least −1, −1.25, or −1.5.
104. The method of any one of the preceding embodiments, wherein the subject in need thereof treated is less likely to require sinus surgery to treat the polyps.

1. A method of treating chronic rhinosinusitis with nasal polyps (CRSwNP) in a subject in need thereof, the method comprising administering a pharmaceutical composition comprising about 100 to about 150 mg of an anti-TSLP-R antibody, or antigen-binding fragment thereof, comprising a heavy chain variable region having the amino acid sequence of SEQ ID NO: 5, and a light chain variable region having the amino acid sequence of SEQ ID NO: 6, to the subject in need thereof.

2. The method of claim 1, wherein the pharmaceutical composition is for administration once every twelve weeks.

3. The method of claim 1, wherein the pharmaceutical composition comprises about 100, about 125, or about 150 mg of verekitug.

4. The method of claim 1, wherein the pharmaceutical composition is for subcutaneous administration.

5. The method of claim 1, wherein verekitug binds to a TSLP receptor (TSLP-R), and wherein verekitug blocks the formation of a heterodimeric TSLP receptor (TSLP-R).

6. (canceled)

7. The method of claim 1, wherein the subject has a history of nasal polyp (NP) surgery or NP exacerbation requiring steroids (e.g., corticosteroids) being administered to the subject sometime in the 24 months preceding the administration of the pharmaceutical composition.

8. The method of claim 1, wherein the subject has an endoscopic nasal polyp score (NPS)≥5.

9. The method of claim 1, wherein the subject has a nasal congestion score (NCS)≥2 over 14 days.

10. The method of claim 1, wherein the subject has CRSwNP symptoms for at least 8 weeks.

11. The method of claim 1, wherein the subject in need thereof has had a stable standard of care for the treatment of CRSwNP for at least 30 days.

12. The method of claim 1, wherein the subject in need thereof has asthma.

13. The method of claim 1, wherein the pharmaceutical composition is for administration of a second dose of verekitug at 12 weeks after a first dose.

14. The method of claim 1, wherein the subject achieves an improvement in at least one of: a nasal polyp score (NPS), a nasal congestion score (NCS), a sinus opacification score (LMK score), a total symptom score (TSS), a difficulty with smell score (DSS score), or a nasal blockage score (NBS).

15. The method of claim 14, wherein the improvement in the nasal polyp score (NPS) is a reduction of at least −1.0, −1.5, −2.0, or −2.5; and wherein the reduction in the nasal polyp score (NPS) is measured at 12 weeks from a first dose, or wherein the reduction in the nasal polyp score (NPS) is measured at 24 weeks from a first dose.

16-17. (canceled)

18. The method of claim 14, wherein the improvement in the nasal congestion score (NCS) is a reduction by at least −1.0, −1.5, or −1.75, and wherein the reduction in the nasal congestion score (NCS) is measured at 24 weeks from a first dose.

19. (canceled)

20. The method of claim 14, wherein the subject achieved the improvement in the nasal congestion score (NCS) at 2 weeks, at 3 weeks, at 4 weeks, at 5 weeks, or at 6 weeks after administration of the pharmaceutical composition.

21. The method of claim 14, wherein the improvement in the sinus opacification score (LMK score) is a reduction from baseline by at least −2, −3, −4, −5, −6, −7, −8, −9, or −10, and wherein the reduction in the sinus opacification score (LMK score) from baseline is measured at 24 weeks from a first dose.

22. (canceled)

23. The method of claim 14, wherein the improvement in the total symptom score (TSS) is a reduction from baseline by at least −7, −8, −9, −10, or −11, and wherein the reduction in the total symptom score (TSS) from baseline is measured at 24 weeks from a first dose.

24. (canceled)

25. The method of claim 14, wherein the improvement in the difficulty with smell score (DSS score) is a reduction from baseline by at least −1, −1.25, or −1.5.

26. The method of claim 14, wherein the improvement in the nasal blockage score (NBS) is a reduction from baseline by at least −1, −1.25, or −1.5.

27. The method of claim 1, wherein the subject in need thereof treated is less likely to require sinus surgery to treat the polyps.

28. The method of claim 1, wherein the pharmaceutical composition comprises:
(i) verekitug at a concentration of about 150 mg/mL to about 300 mg/mL;
(ii) a pharmaceutically acceptable buffer at a concentration of about 5 mmol/L to about 100 mmol/L;
(iii) glycine or a pharmaceutically acceptable salt thereof, at a concentration of 150 mmol/L to about 200 mmol/L, or proline or a pharmaceutically acceptable salt thereof, at a concentration of 150 mmol/L to about 200 mmol/L; and
(iv) a surfactant at a concentration of about 0.001% to about 1% (w/v).

29. The method of claim 28, wherein the pharmaceutically acceptable buffer is:
histidine buffer at a concentration of 18 mmol/L to 22 mmol/L; glycine, or a pharmaceutically acceptable salt thereof, at a concentration of 160 mmol/L to 200 mmol/L; the surfactant is polysorbate 80 at a concentration of 0.01% (w/v) to 0.05% (w/v); and the pharmaceutical composition is at a pH of about 5.6 to 5.8;
histidine buffer at a concentration of 20 mmol/L: glycine, or a pharmaceutically acceptable salt thereof, at a concentration of 180 mmol/L: the surfactant is polysorbate 80 at a concentration of 0.03% (w/v), and the pharmaceutical composition is at a pH of about 5.7;
histidine buffer at a concentration of 18 mmol/L to 22 mmol/L; proline, or a pharmaceutically acceptable salt thereof, at a concentration of 160 mmol/L to 200 mmol/L: the surfactant is polysorbate 80 at a concentration of 0.01% (w/v) to 0.05% (w/v); and the pharmaceutical composition is at a pH of about 5.6 to 5.8; or
histidine buffer at a concentration of 20 mmol/L; proline, or a pharmaceutically acceptable salt thereof, at a concentration of 180 mmol/L: the surfactant is polysorbate 80 at a concentration of 0.02% (w/v); and the pharmaceutical composition is at a pH of about 5.7.

30-33. (canceled)

34. The method of claim 1, wherein the anti-TSLP-R antibody, or antigen-binding fragment thereof, comprises a heavy chain having an amino acid sequence of SEQ ID NO: 1; and
a light chain having an amino acid sequence of SEQ ID NO: 3.


SEQUENCE LISTING (12 sequences)

SEQ ID NO: 1 - protein, 448 aa, synthetic construct
EVQLLESGGGLVQPGGSLRLSCAASGFTFRSSAMHWVRQAPGKGLKWVSSVSGSGAGTYYADSVKGRFTISRDNPKNTLYLQMNSLRAEDTAVYYCVKEGGSRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - RNA, 1347 nt, synthetic construct
  source 1..1347: mol_type = other RNA
gaggtgcagctgttggagtctgggggaggcttggtacagccgggggggtccctgagactctcctgtgcagcctctggattcacctttcgcagctctgccatgcattgggtccgccaggctccagggaagggactgaaatgggtctcaagtgttagtggcagtggtgctggaacatactacgcagactccgtgaagggccggttcaccatctccagagacaatcccaagaatacactgtatctgcaaatgaacagtctgagagccgaggacacggccgtatattattgtgtgaaagaagggggcagccggggttttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactcccttagtagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - protein, 214 aa, synthetic construct
DIQMTQSPSSLSASVGDRVTITCRASQDISNYLAWFQQKPGKAPKSLIYTASSLQSGVPSKFSGSGSGTDFTLTISSLQPEDFATYYCQQYNLYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - RNA, 645 nt, synthetic construct
  source 1..645: mol_type = other RNA
gacatccagatgacccagtctccatcctcactgtctgcatctgtaggagacagagtcaccatcacttgtcgggcgagtcaggacattagcaattatttagcctggtttcagcagaaaccagggaaagcccctaagtccctgatctatactgcatccagtttgcaaagtggggtcccatcaaagttcagcggcagtggatctgggacagatttcactctcaccatcagcagcctgcagcctgaagattttgcaacttattactgccaacagtataatctttatcctccgacgttcggccaagggaccaaggtggaaatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 5 - protein, 118 aa, synthetic construct
EVQLLESGGGLVQPGGSLRLSCAASGFTFRSSAMHWVRQAPGKGLKWVSSVSGSGAGTYYADSVKGRFTISRDNPKNTLYLQMNSLRAEDTAVYYCVKEGGSRGFDYWGQGTLVTVSS

SEQ ID NO: 6 - protein, 107 aa, synthetic construct
DIQMTQSPSSLSASVGDRVTITCRASQDISNYLAWFQQKPGKAPKSLIYTASSLQSGVPSKFSGSGSGTDFTLTISSLQPEDFATYYCQQYNLYPPTFGQGTKVEIK

SEQ ID NO: 7 - protein, 5 aa, synthetic construct
SSAMH

SEQ ID NO: 8 - protein, 17 aa, synthetic construct
SVSGSGAGTYYADSVKG

SEQ ID NO: 9 - protein, 9 aa, synthetic construct
EGGSRGFDY

SEQ ID NO: 10 - protein, 11 aa, synthetic construct
RASQDISNYLA

SEQ ID NO: 11 - protein, 7 aa, synthetic construct
TASSLQS

SEQ ID NO: 12 - protein, 9 aa, synthetic construct
QQYNLYPPT